(19) United States
(12) Patent Application Publication
(10) Pub. No.: US 2020/0009118 A1
(43) Pub. Date: Jan. 9, 2020

(54) COMPOSITIONS AND METHODS FOR THE TREATMENTOF NEURODEGENERATIVE AND OTHER DISEASES

(71) Applicant: Glialogix, Inc., Sunnyvale, CA (US)

(72) Inventors: Thaddeus Cromwell Reeder, San Carlos, CA (US); Mark W. Moore, San Anselmo, CA (US); David K. Lyon, Bend, OR (US); Casey K. Jager, Bend, OR (US); Doug Lorenz, Bend, OR (US); Corey Bloom, Bend, OR (US); Kimberley Shepard, Bend, OR (US)

(73) Assignee: Glialogix, Inc., Sunnyvale, CA (US)

(21) Appl. No.: 16/270,022

(22) Filed: Feb. 7, 2019

Related U.S. Application Data
(63) Continuation of application No. 15/789,243, filed on Oct. 20, 2017, now abandoned.

(51) Int. Cl. A61K 31/4402; A61P 25/28; A61P 35/00; A61K 31/353; A61K 47/32

(52) U.S. Cl. CPC A61K 31/4402; A61P 25/28; A61K 2300/00; A61K 31/353; A61K 47/32; A61P 35/00

(57) ABSTRACT

In one embodiment, the present application discloses methods of treating diseases and disorders with sulfasalazine, an ABCG2 inhibitor and pharmaceutical formulations of sulfasalazine where the bioavailability of the sulfasalazine is increased. In another embodiment, the present application also provides dosing regimens for treating neurodegenerative diseases and disorders with compositions comprising sulfasalazine and an ABCG2 inhibitor.

### CROSS-REFERENCE TO RELATED APPLICATION

This application claims the priority under 35 U.S.C. 119(e) of U.S. Application No. 62/411,512, filed Oct. 21, 2016, which is incorporated into this application by reference.

### FIELD OF THE INVENTION

The invention relates generally to the field of pharmaceuticals, pharmaceutical formulations, methods of treatment using such formulations, formulations for use in treating patients and in particularly compositions, formulations, uses and methods for treating patients with neurological diseases wherein the formulation comprises an amorphous sulfasalazine, a polymer, and an ABCG2 inhibitor.

### BACKGROUND OF THE INVENTION

Neurodegenerative diseases are collectively a leading cause of death and disability. While the ultimate causes and natural histories of the individual neurodegenerative diseases differ, common pathological processes occur in most, if not all, neurodegenerative diseases. These common pathological processes include high levels of activated glial cells (“neuroinflammation”), dysregulated glutamate signaling and chronic damage to axons and neurons.

Progressive multiple sclerosis (P-MS) is a devastating neurodegenerative disease that affects approximately 120,000 people in the United States and 350,000 people in the developed world. P-MS patients progressively accumulate disabilities, including changes in sensation (hypoesthesia), muscle weakness, abnormal muscle spasms, or difficulty moving; difficulties with coordination and balance; problems in speech (dysarthria) or swallowing (dysphagia), visual problems (nystagmus, optic neuritis, phosphenes or diplopia), fatigue and acute or chronic pain syndromes, bladder and bowel difficulties, cognitive impairment, or emotional symptomatology (mainly major depression). The only drug currently approved to treat P-MS in the United States is mitoxantrone (Novantrone), a cytotoxic agent that is also used to treat cancers. Mitoxantrone has a serious adverse effect profile and carries a lifetime limit on exposure. The treatment of P-MS remains a significant unmet medical need.

There are three major sub-types of P-MS recognized by the National Multiple Sclerosis Society (US): Primary Progressive Multiple Sclerosis (PP-MS), Secondary Progressive Multiple Sclerosis (SP-MS) and Progressive-Relapsing Multiple Sclerosis (PR-MS). Approximately 85% of multiple sclerosis patients clinically present with Relapse Remitting Multiple Sclerosis (RR-MS), characterized by episodes of acute neurological deficits (relapses), followed by partial or complete recovery of the deficits. After a median time to conversion of around 19 years, approximately 70% of RR-MS patients develop a progressive neurological decline, clinically recognized as SP-MS. Approximately 10% of multiple sclerosis patients clinically present with PP-MS, characterized by a progressive neurological decline with few to no preceding episodes of neurological deficits (relapses), while 5% present with PR-MS, characterized by a steady worsening disease from the onset but also have clear acute flare-ups (relapses), with or without recovery, e.g. Compton et al, Lancet 372:1502-1517 (2008); Trapp et al, Annu. Rev. Neurosci. 31:247-269 (2008). Here, PP-MS, SP-MS and PR-MS are grouped together as P-MS, as they share many similarities, including natural history, clinical manifestations and pathology, e.g. Kremenchutsky et al, Brain 129:584-594 (2006); Lassmann et al, Nat. Rev. Neurology 8:647-656 (2012); Stys et al, Nat. Rev. Neuroscience 13:507-514 (2012).

Thus far, drugs that are effective for RR-MS have limited efficacy in P-MS, e.g. Fox et al, Multiple Sclerosis Journal 18:1534-1540 (2012). This is believed to be due to current RR-MS drugs primarily targeting the peripheral immune system (B and T-cells) while P-MS is instead primarily driven by resident CNS inflammatory cells, including microglia and astrocytes, e.g. Fitzner et al, Curr. Neuropharmacology 8:305-315 (2008); Weiner, J. Neurology 255, Suppl. 1:3-11 (2008); Lassman Neurology 8:647-656 (2012). Recent evidence suggests that the efficacy of mitoxantrone in P-MS may be due to inhibition of activation of astrocytes, thereby linking anti-neuroinflammation with efficacy in P-MS, e.g. Burns et al, Brain Res. 1473: 236-241 (2012).

In addition to resident CNS neuroinflammation, P-MS is also accompanied by demyelination, loss of axons and ultimately death of neuronal cells. The mechanisms that drive demyelination and axonal and neuronal damage are not completely understood, although glutamate excitotoxicity is one of the leading suspects in human P-MS, e.g. Frigo, Curr. Medicin. Chem. 19:1295-1299 (2012). In particular, oligodendrocytes—the cells responsible for producing myelin—are especially sensitive to elevated levels of glutamate, e.g. Matute, J. Anatomy 219:53-64 (2011). Subsets of MS patients have been demonstrated to have elevated extracellular glutamate levels in the cerebrospinal fluid, e.g. Sarchielli et al, Arch. Neurol. 60:1082-1088 (2003) and P-MS patients have an increased incidence of seizures and neuropathic pain; both conditions may derive from excessive glutamate signaling and are treated clinically with anti-glutamatergics, e.g. Eriksson et al, Mult. Scler. 8:495-499 (2002); Svendsen et al, Pain 114: 473-481 (2004).

Another neurodegenerative disease thought to involve excessive glutamatergic signaling is amyotrophic lateral sclerosis (ALS), which affects approximately 100,000 patients in the developed world. ALS patients progressively lose motor neuron function, causing muscular atrophy, paralysis and death. The average lifespan after diagnosis is only 3-5 years. Riluzole (Rilutek) is the only known treatment that has been found to improve survival in ALS patients; however, the treatment is effective only to a modest extent by lengthening the survival time by only several months. Thus treatment of ALS remains a significant unmet medical need.

At the molecular level, ALS is characterized by excessive glutamatergic signaling leading to neuroexcitotoxicity and motor neuron death; see, e.g. Bogaert et al, CNS Neurol. Disord. Drug Targets 9:297-304 (2010). Affected tissues in the spinal cord also have high levels of activated microglia and activated astrocytes, collectively recognized as neuroinflammation; see, e.g. Philips et al, Lancet Neurol. 10:253-263 (2011) and neuroinflammatory cells have been shown to drive disease progression in ALS animal models; see, e.g. Ilieva et al, J. Cell Biol. 187: 761-772 (2009). The glutamate pathway has been clinically validated in ALS, as Riluzole inhibits multiple glutamate activities, including the activity of AMPA glutamate receptor; see, e.g., Lin et al, Pharmacology 85:54-62 (2010).

Approximately 10% of ALS cases are familial, while the remainders are believed to be sporadic, with no clear genetic cause to date. Among the familial cases, approximately 20% are due to mutations in the SOD1 gene. Mice and rats genetically altered to contain the mutant human SOD1 gene develop motor neuron disease that phenotypically resembles human ALS. Because of this, most potential ALS therapies are tested in the SOD1 mouse or rat model for efficacy.

Excessive glutamatergic signaling is believed to play a causal role in neurodegenerative diseases besides P-MS and ALS. For instance, neuropathic pain is a chronic condition caused by damage or disease that affects the somatosensory system. Neuropathic pain is associated with neuronal hyperexcitability, a common consequence of excessive glutamate signaling, see, e.g. Baron et al, Lancet Neurology 9: 807-819 (2010). Neuropathic pain may manifest in abnormal sensations called dysesthesia and pain produced by normally non-painful stimuli (allodynia). Neuropathic pain may have continuous and/or episodic (paroxysmal) components. The latter are likened to an electric shock. Common qualities include burning or coldness, “pins and needles” sensations, numbness and itching. Neuropathic pain is clinically treated with compounds that possess anti-glutamatergic activity (e.g. Topamax, Pregabalin). Importantly, sulfasalazine has previously been shown to have efficacy in models of diabetic neuropathy (e.g. Berti-Mattera et al, Diabetes 57: 2801-2808 (2008); U.S. Pat. No. 7,964,585) and cancer-induced bone pain (e.g. Ungardet et al, Pain 155: 28-36 (2014)).

Epilepsy and other seizure disorders are also associated with neuronal hyperexcitability. Notably, multiple drugs used to treat seizure disorders reduce glutamatergic activity, including carbamazepine, lamotrigine, levitiracetam, phenytoin, topiramate and pregabalin. Fifty million people in the world have seizure disorders, and a third of these patients have seizures that are resistant to current therapies, with brain surgery often being the only medical option for some of these patients. In particular, there are a large number of pediatric and juvenile epilepsies that remain poorly treated. The initiating events resulting in pediatric and juvenile seizure disorders are diverse and include genetic abnormalities (e.g. Angelman Syndrome, Ring Chromosome 20 Syndrome and CDKL5 Disorder) and infection and trauma (e.g. Rasmussen's Syndrome, traumatic brain injury). In many cases, the initiating events that result in the seizure disorder are not well understood. Recent work, however, has found that many childhood and pediatric seizure disorders result in a common neuroinflammatory pathology within the brain, observed by high levels of activated astrocytes and microglia, e.g. Choi et al, J of Neuroinflammation 6:38-52. As a consequence of ineffective treatment, patients with drug-resistant epilepsy have increased risks of premature death, injuries, psychosocial dysfunction, and a reduced quality of life, e.g. Kwan et al. N Engl J Med 365:919-26. Work described herein demonstrates that expression and activity of xCT in astrocytes and microglia is upregulated by a large and diverse number of agents known to cause and/or reflect activation of neuroinflammatory cells and/or to cause or reflect damage to neurons, axons and/or oligodendrocytes. Thus, the expression profile of xCT matches the neuroinflammatory pathology observed in many epilepsies and seizure disorders. Previous work has also shown that xCT plays a role in seizures that can accompany glioblastoma multiforme (GBM) and that sulfasalazine has efficacy against seizures in GBM mouse models (e.g. Buckingham et al, Nat Med. 17:1269-1274 (2011)).

Other neurodegenerative diseases where compounds with anti-glutamatergic activity are used clinically include Parkinson's disease (amantadine and budipine) and Alzheimer's disease (Memantine). Anti-glutamatergics are being investigated for treatment of traumatic brain injury, Huntington's disease, multiple sclerosis, and ischemic stroke. In many cases, these neurological diseases are also accompanied by high levels of neuroinflammation. Other neurological diseases that are linked to excessive glutamate signaling and neuroinflammation include Rett Syndrome, Frontotemporal Dementia, HIV-associated Dementia and Alexander disease.

The system x_{c}⁻ glutamate-cysteine exchange transporter (herein “system x_{c}⁻”) is the only glutamate transporter that normally functions to release glutamate into the extracellular space. The amount of glutamate released by system x_{c}⁻ is sufficient to stimulate multiple ionotropic and metabotropic glutamate receptors in vivo. Current anti-glutamatergics target either the vesicular release of glutamate or individual glutamate receptors that lie downstream of glutamate release (e.g., riluzole to the AMPA receptor). In contrast, system x_{c}⁻ is responsible for the non-vesicular release of glutamate and lies upstream of the individual glutamate receptors. The protein xCT (SLC7A11) is the only currently identified catalytic component of system x_{c}⁻.

Sulfasalazine (also referred to as 2-hydroxy-5-[(E)-2-{4-[(pyridin-2-yl) sulfamoyl] phenyl}diazen-1-yl]benzoic acid, 5-([p(2-pyridylsulfamoyl) phenyl]azo) salicylic acid or salicylazosulfapyridine) is a conjugate of 5-aminosalicylate and sulfapyridine, and is widely prescribed for inflammatory bowel disease, rheumatoid arthritis, and ankylosing spondylitis. Sulfasalazine is degraded by intestinal bacteria into its metabolites, 5-aminosalicylate and sulfapyridine. The mechanism of action in inflammatory bowel disease and rheumatoid arthritis is unknown, although action in the colon may be mediated by a metabolite, 5-aminosalicylate. Sulfasalazine has been shown to be an inhibitor of system x_{c}⁻.

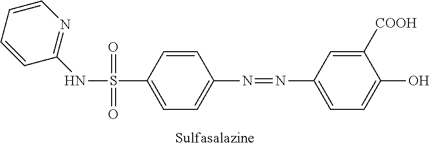 [SMILES: O=C(O)c1cc(N=Nc2ccc(S(=O)(=O)Nc3ccccn3)cc2)ccc1O]

The current U.S. on-market formulations of sulfasalazine (e.g. Azulfidine®) have poor bioavailability, with only approximately 15% or less of the compound reaching the circulation following oral dosing (see, for example, Label for Azulfidine® sulfasalazine tablets, USP). A major toxicity concern is exposure of the gastrointestinal tract to sulfasalazine, where it causes nausea, diarrhea and cramping in a dose-dependent manner, see e.g. Weaver, J. Clin. Rheumatol. 5: 193-200 (1999). An additional toxicity concern is sulfapyridine, one of the metabolites of sulfasalazine. Sulfapyridine is highly (>70%) bioavailable and is believed to be produced by intestinal bacteria, see, e.g., Peppercorn, M., J. Clin. Pharmacol. 27: 260-265 (1987); Watkinson, G., Drugs 32: Suppl 1:1-11 (1986). The poor oral bioavailability and toxicities of sulfasalazine are even more problematic when treating neurological diseases, as the level in the CNS is less than the systemic level (see FIG. 11), necessitating a higher dose to maintain effective drug coverage in the CNS compared to systemically.

US20140221321A1 discloses one method to increase the oral bioavailability of sulfasalazine by developing an amorphous composition of sulfasalazine with increased solubility at enteric pH. Work in humans has identified another potential mechanism to increase the oral bioavailability of sulfasalazine. Yamasaki et al (Clinical Pharmacology Therapeutics 84: 95-103) demonstrated that people with alleles of the efflux transporter ABCG2 that were associated with high efflux activity had lower plasma levels of sulfasalazine following oral administration of the on-market (crystalline) formulation.

### SUMMARY OF THE INVENTION

An aspect of the invention is a formulation comprising a therapeutically effective amount of an amorphous sulfasalazine, a pharmaceutically acceptable carrier which is presently in the form of a polymer; and an ACBG2 inhibitor.

Another aspect of the invention is the use of the formulation as described above by itself or in combination with other pharmaceutically active drugs in treating a neurodegenerative disease of a human patient which diseases are described herein.

The present application provides methods for targeting system x_{c}⁻ as a therapeutic approach to diseases involving excessive glutamatergic signaling. The present application discloses experiments demonstrating that expression and activity of system x_{c}⁻ is induced in microglia and astrocytes by agents known to cause or reflect damage to neurons, axons and oligodendrocytes, thereby: (1) linking system x_{c}⁻ to excessive glutamatergic signaling in multiple neurodegenerative diseases and (2) xCT over-expression to a neuroinflammatory phenotype present in many neurodegenerative diseases. The present application also discloses the administration of an inhibitor of system x_{c}⁻, such as sulfasalazine, to treat neurodegenerative diseases involving excessive glutamatergic signaling, such as P-MS and ALS. Without being bound by any theory asserted herein, the working hypothesis is that system x_{c}⁻, by releasing excessive amounts of glutamate, causes neuronal damage, thereby activating neuroinflammatory cells. This in turn elevates levels of system x_{c}⁻, causing a positive feedback loop that damages and ultimately kills axons and neurons, including motor neurons. Inhibiting system x_{c}⁻ with an inhibitor such as sulfasalazine can interrupt this feedback loop and can reduce damage to the axons and neurons, including motor neurons.

In one aspect, the present application provides methods of treatment of various diseases using system x_{c}⁻ inhibitors, including methods using improved dosing regimens. In addition, the present application describes formulations of a system x_{c}⁻ inhibitor, sulfasalazine, where the formulations increase the bioavailability of orally-administered sulfasalazine. Those formulations can be used in the treatment of neurodegenerative diseases and disorders as well as other diseases and disorders, including rheumatoid arthritis and ankylosing spondylitis (diseases for which sulfasalazine is currently approved in various markets).

Experiments described herein using a mouse models of neurodegeneration demonstrate that treatment with sulfasalazine significantly: (1) reduces levels of neuroinflammatory cells in the spinal cord (see Example 3), including both activated microglia and activated astrocytes, (2) increases the absolute survival and the survival after onset of definitive neurological disease in the SOD1 mouse model of ALS (Example 1); and (3) prevent demyelination in a mouse model of optic neuritis (Example 16). Thus, in various embodiments, the present invention provides methods for treating P-MS, ALS, and other neurodegenerative diseases by administering to the patient a pharmaceutical composition comprising a therapeutically effective amount of sulfasalazine and a pharmaceutically acceptable excipient. In certain embodiments, methods are provided for treating other neurodegenerative diseases involving excessive glutamatergic signaling comprising administering to the patient with such a neurodegenerative disease a pharmaceutical composition comprising a therapeutically effective amount of sulfasalazine and a pharmaceutically acceptable excipient, wherein the neurodegenerative disease is selected from Parkinson's disease, Alzheimer's disease, epilepsy and other seizure disorders, neuropathic pain, traumatic brain injury, Huntington's disease, ischemic stroke, Rett Syndrome, Frontotemporal Dementia, HIV-associated Dementia and Alexander disease. Increasing the Bioavailability of Sulfasalazine:

One challenge with treating P-MS, ALS and other diseases with pharmaceutical compositions comprising sulfasalazine is the poor oral bioavailability of the standard formulations of sulfasalazine. For example, only 15% or less of the sulfasalazine in an orally administered dose of Azulfidine is absorbed into the bloodstream (see Azulfidine Sulfasalazine Tablets Label, LAB-0241-3.0, revised October 2009). Because the level of sulfasalazine at the sites of action relevant to neurodegenerative diseases (such as the spinal cord) is proportional to the amount of sulfasalazine in the plasma (Example 4), the poor bioavailability of the current oral formulation of sulfasalazine limits the amount of sulfasalazine that reaches such sites of action. Thus, use of a standard formulation of sulfasalazine to treat neurodegenerative diseases would require large oral doses of sulfasalazine to be administered. This would expose patients to high levels of sulfasalazine in the gastrointestinal tract and generate high levels of sulfapyridine in the plasma, thereby increasing toxicity. The present application addresses these issues, among others, by improving the oral bioavailability of sulfasalazine for the treatment of seizure disorders, P-MS, ALS or other diseases, including non-neurodegenerative diseases. Increasing such bioavailability would allow dosing levels of sulfasalazine to be lower, with the further benefit of limiting gastrointestinal exposure to sulfasalazine and systemic exposure to sulfapyridine. In one aspect, there is provided a method for limiting gastrointestinal exposure to sulfasalazine and systemic exposure to sulfapyridine by the administration of a therapeutically effective amount of the pharmaceutical composition as disclosed herein. The formulations disclosed may increase the therapeutic index for sulfasalazine in the treatment of various diseases. The application provides methods of treating various diseases and disorders using the compositions in which the solubility and/or bioavailability of sulfasalazine has been increased. In certain embodiments, there are provided methods for treating a disease or disorder in a patient comprising orally administering to the patient a liquid pharmaceutical composition or a solid pharmaceutical composition comprising a therapeutically effective amount of sulfasalazine and an ABCG2 inhibitor. In one embodiment, the present application discloses a method for treating a patient with a seizure disease or disorder, the method comprising orally administering to the patient a pharmaceutical composition comprising a therapeutically effective amount of sulfasalazine, an ABCG2 inhibitor, optionally a polymer, and a pharmaceutically acceptable excipient. In one aspect, the sulfasalazine is in an essentially amorphous form. In another aspect, the seizure disease or disorder is selected from the group consisting of Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia, epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia, epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs).

Previous work has demonstrated that sulfasalazine treatment can diminish the growth of tumors, including brain tumors, see Sontheimer, H. et al. Expert Opin. Investig. Drugs 21: 575-578 (2012) and Polewski, M, et al. Mol. Cancer Res. 14: 1229-1242 (2016). Using the on-market, crystalline formulation of sulfasalazine, dosing in brain cancer patients was limited by toxicities, including nausea, dysphagia and neutropenia, see Takeuchi, S. et al. Neurology India 62:42-47 (2014). In another aspect, the present application discloses a method for treating a patient with cancer, the method comprising orally administering to the patient a pharmaceutical composition comprising a therapeutically effective amount of sulfasalazine, an ABCG2 inhibitor, optionally a polymer, and a pharmaceutically acceptable excipient. In one aspect, the sulfasalazine is in an essentially amorphous form. In another aspect, the cancer is selected from the group selected from astrocytoma, glioma, glioblastoma and long-term epilepsy associated tumors (LEATs) for example ganglioglioma, oligodendroglioma, and dysembryoplastic neuroepithelial tumors (DNETs).

In one aspect of the method, the neurodegenerative diseases include progressive multiple sclerosis and other demyelinating diseases, including Acute Disseminated Encephalomyelitis, Adrenoleukodystrophy, Adrenomyeloneuropathy, Chronic Axonal Neuropathy, Chronic Inflammatory Demyelinating Polyneuropathy or CIDP, Chronic Relapsing Polyneuropathy, Devic Disease, Guillian-Barre Syndrome, HIV induced CIDP, Leber's Hereditary Optic Neuropathy, Lewis Sumner variant of CIDP, Multifocal Acquired Demyelinating Sensory and Motor Neuropathy, Multifocal Motor Neuropathy, Neuromyelitis Optica, Optic Neuritis, Paraproteinaemic Demyelinating Neuropathy, Tropical Spastic Paraparesis, amyotrophic lateral sclerosis, Alzheimer's disease, Parkinson's disease, epilepsy and other seizure disorders, including but not limited to Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, epileptic encephalopathies, Focal Cortical Dysplasia, and Tuberous Sclerosis Complex, neuropathic pain, Huntington's disease, ischemic stroke, traumatic brain injury, concussion, Rett Syndrome, Frontotemporal Dementia, HIV-associated Dementia Alexander disease and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs).

The term ABCG2 inhibitor is an acronym for ATP-binding cassette sub-family G member 2. ATP-binding cassette sub-family G member 2 is a protein that in humans is encoded by the ABCG2 gene, see Allikmets R, et al. Hum Mol Genet. 5: 1649-55 (1997) and Doyle L. et al. Proc Natl Acad Sci USA. 95: 15665-70 (1999). ABCG2 has also been designated as CDw338 (cluster of differentiation w338). The membrane-associated protein encoded by this gene is included in the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). ABCG2 protein is a member of the White subfamily. Alternatively referred to as the Breast Cancer Resistance Protein, this protein functions as a xenobiotic transporter which may play a role in multi-drug resistance to chemotherapeutic agents including mitoxantrone and camptothecin analogues.

Examples of ABCG2 inhibitors include the following: N-[4-[2-(3,4-Dihydro-6,7-di methoxy-2(1H)-isoquinolinyl)ethyl]phenyl]-9,10-dihydro-5-methoxy-9-oxo-4-acridinecarboxamide (elecridar); 2-chloro-N-(4-chloro-3-(pyridin-2-yl)phenyl)-4-(methylsulfonyl)benzamide (HhAntag691); (3S,6S,12aS)-1,2,3,4,6,7,12,12a-Octahydro-9-methoxy-6-(2-methylpropyl)-1,4-dioxopyrazino[1′,2′:1,6]pyrido[3,4-b]indole-3-propanoic acid 1,1-dimethylethyl ester (raltegravir); N-(4-Methyl-3-((4-(pyridin-3-yl)pyrimidin-2-yl)amino)phenyl)-4-((4-methylpiperazin-1-yl)methyl)benzamide (imatinib); Fumitremorgin C; 4-[4-[[4-chloro-3-(trifluoromethyl)phenyl]carbamoylamino]phenoxy]-N-methylpyridine-2-carboxamide; 4-methylbenzenesulfonic acid (sorafenib); (1E,6E)-1,7-bis (4-hydroxy-3 methoxyphenyl)-1,6-heptadiene-3,5-dione (curcumin) and Cathomycin sodium. The polymer used in a formulation of the invention must be biocompatible, pharmaceutically acceptable and water soluble. The polymer may be a copolymer of vinylpyrrolidone with vinyl acetate and as such can be any PVP VA polymer that is water soluble including PVP VA64.

In another aspect of the above methods, the ABCG2 inhibitor is selected from the group consisting of TPGS, polysorbate (Tween) and Pluronic. In another aspect, the ABCG2 inhibitor is TPGS. In one variation, the ABCG2 inhibitor is a non-ionic compound. In another variation, the ABCG2 inhibitor is a GRAS compound. In another variation, the ABCG2 is selected from the group consisting of TPGS, Tocophersolan, and polysorbate, polysorbate-20 (Tween-20), Brij30, Cremphor EL, and Pluronic compounds, Pluronic P85 and Pluronic L21. In another aspect, the pharmaceutical formulation is a solid dose formulation, wherein the formulation comprises a polymer selected from PVP VA64 or HPMCAS. In another aspect, the pharmaceutical formulation is a liquid formulation that does not comprise a polymer such as PVP VA64 or HPMCAS. In another aspect, the formulation comprises between 1 mg and 2,000 mg of the ABCG2 inhibitor, such as TPGS per dose, such as 10 mg, 100 mg, 200 mg, 300 mg, 400 mg, 500 mg, 750 mg, 1000 mg, 1,500 mg or 2,000 mg. In another aspect, the ratio of the sulfasalazine to PVP VA64 or HPMCAS in the pharmaceutical composition is about 20:80 wt/wt to 50:50 wt/wt, or about 25:75 wt/wt. In another aspect, the in vitro solubility of the sulfasalazine is at least 500 μg/ml. In yet another aspect, the in vitro solubility of the sulfasalazine is between about 500 μg/ml and 12,000 μg/ml, or higher.

In another embodiment, there is provided a pharmaceutical composition comprising a therapeutically effective amount of sulfasalazine, an ABCG2 inhibitor, optionally a polymer, and a pharmaceutically acceptable excipient, wherein the sulfasalazine is in an essentially amorphous form. In one aspect, the ABCG2 inhibitor is selected from the group consisting of TPGS, Tween and Pluronic. In another aspect, the ABCG2 inhibitor is TPGS. In yet another aspect of the formulation, the pharmaceutical formulation is a solid dose formulation or a liquid dose formulation, wherein the formulation comprises a polymer selected from PVP VA64 or HPMCAS. In another aspect, the pharmaceutical formulation is a solid formulation and the ratio of the sulfasalazine to the ABCG2 inhibitor is from about 1:9 to 200:1 wt/wt, and the ratio of the sulfasalazine to PVP VA64 or HPMCAS in the pharmaceutical composition is about 20:80 wt/wt to 50:50 wt/wt, or about 25:75 wt/wt. In one variation, the ratio of the sulfasalazine to the ABCG2 inhibitor is from about 1:5, 1:3, 1:2, 1:1, 10:1, 20:1, 30:1, 40:1, 50:1, 60:1, 70:1, 80:1, 90:1, 100:1; 125:1, 150:1, 175:1 or 200:1 wt/wt.

In another embodiment, there is provided a method for increasing the oral bioavailability of pharmaceutical composition comprising sulfasalazine and a pharmaceutically acceptable polymer by at least 1.5 to 250 fold, such as an increase of about 5 fold, 10 fold, 15 fold, 20 fold or about 25 fold (or 25 times), the method comprising formulating the pharmaceutical composition with an ABCG2 inhibitor selected from the group consisting of TPGS (Tocophersolan) and Tween-20 (polysorbate 20), Brij30, Cremphor EL, Pluronic P85 and Pluronic L21, wherein the sulfasalazine is in an amorphous form and the ratio of the sulfasalazine to the ABCG2 inhibitor is from about 1:9 to 200:1 wt/wt, and as disclosed above. In one aspect of the method, the ABCG2 inhibitor is TPGS. In another aspect, the pharmaceutically acceptable polymer is PVP VA64 or HPMCAS.

Accordingly, the application discloses a method of increasing the oral bioavailability of pharmaceutical composition, comprising: combining an amorphous form of sulfasalazine with an ABCG2 inhibitor selected from the group consisting of TPGS (Tocophersolan) and Tween-20 (polysorbate 20), Brij30, Cremphor EL, Pluronic P85 and Pluronic L21, wherein the ratio of the sulfasalazine to the ABCG2 inhibitor is from about 1:3 to 1:7 wt/wt, whereby oral bioavailability of the sulfasalazine in the composition is increased by 200% or more relative to the oral bioavailability of sulfasalazine alone. In one variation, the ABCG2 inhibitor is TPGS. In another variation, the pharmaceutically acceptable polymer is PVP VA64 or HPMCAS.

In certain embodiments, this application discloses pharmaceutical compositions comprising sulfasalazine and an inhibitor of the ABCG2 efflux transporter (i.e., ABCG2 efflux inhibitors or ABCG2 inhibitors), wherein the compositions are used to treat neurodegenerative diseases and disorders. In one aspect, the ABCG2 efflux inhibitors is selected from the group consisting of Pluronic P85, Tween 20, E-TPGS (TPGS, and as defined herein), Pluronic 85, Brij 30, Pluronic L81, Tween 80 and PEO-PPO, or mixtures thereof. In another aspect, the ABCG2 inhibitor is TPGS or Tween 20, or a mixture thereof. In another aspect, the ABCG2 inhibitor is TPGS. In one variation, the composition comprises of one ABCG2 inhibitor, or a mixture of two or more ABCG2 inhibitors.

In certain embodiments, the presence of an ABCG2 inhibitor increases the oral bioavailability of sulfasalazine by at least 25%, at least 50%, at least 100%, at least 150%, at least 200%, at least 250%, at least 300%, at least 500%, at least 1000%, at least 2000%, at least 6,000%, at least 8,000%, at least 10,000%, at least 12,000%, at least 15,000%, at least 20,000%, at least 25,000% or at least 28,000% higher than the plasma level of sulfasalazine after administration of the same dose level of crystalline sulfasalazine, as measured in the blood plasma. In one embodiment, the compositions comprising sulfasalazine and the ABCG2 inhibitor comprises a solid oral dose. In another embodiment, the sulfasalazine that is in the solid oral dose is in an amorphous state; in other embodiments, the sulfasalazine is in a crystalline state. In other embodiments, the sulfasalazine and the ABCG2 inhibitor comprises a liquid suspension or solution. In certain embodiments, the ABCG2 inhibitor comprises 0.01% to 90%, such as 0.1%, 0.5%, 1%, 5%, 10%, 20%, 30%, 40%, 50%, 60%, 70%, 80% and 90% by weight of the total composition. In certain embodiments, the ABCG2 inhibitor comprises 0.01% to 90%, such as 0.1%, 0.5%, 1%, 5%, 10%, 20%, 30%, 40%, 50%, 60%, 70%, 80% and 90% by weight relative to sulfasalazine (i.e., ABCG2 inhibitor:sulfasalazine) in the therapeutic composition. In one aspect, the sulfasalazine is in amorphous form.

In certain embodiments, the present application discloses pharmaceutical compositions comprising sulfasalazine in a formulation suitable for intravenous (IV) dosing. In one aspect, the IV formulation contains an ABCG2 inhibitor. These formulations are suitable for acute care treatment, especially for treatment of ischemic stroke, traumatic brain injury, seizure disorders and demyelinating diseases.

In one embodiment, the pharmaceutical composition is formulated such that administration, such as oral administration or IV administration, of the formulated pharmaceutical composition results in a plasma level of sulfasalazine 30 minutes after such administration that is at least 25%, at least 50%, at least 100%, at least 150%, at least 200%, at least 250% at least 300%, at least 500%, at least 1,000%, at least 2,000% or at least 8,000% higher than the plasma level of sulfasalazine 30 minutes after administration of the same dose level of crystalline sulfasalazine. In certain embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 30 minutes after such administration that is about 25%, about 50%, about 100%, about 150%, about 200%, about 250%, about 300%, about 500%, about 1,000%, about 2,000%, about 8,000%, about 10,000% or about 25,000% higher than the plasma level of sulfasalazine 30 minutes after administration of the same dose level of crystalline sulfasalazine. In yet other embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 30 minutes after such administration that is between about 25% and 25,000%, about 75% and 10,000%, or about 100% and 1,000%, inclusive, higher than the plasma level of sulfasalazine 30 minutes after administration of the same dose level of crystalline sulfasalazine. In certain of the embodiments, the disease or disorder is a neurodegenerative disease or disorder, such as P-MS or ALS, and seizure disorders, including Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In certain embodiments, the disease or disorder is selected from neuropathic pain, such as neuropathic pain results from painful diabetic neuropathy, or neuropathic pain manifests as dysesthesia, or neuropathic pain manifests as allodynia; rheumatoid arthritis or ankylosing spondylitis. In certain embodiments, the pharmaceutical composition is in an oral dosage form, in a spray dried dispersion form or in an IV form.

In another embodiment, the pharmaceutical composition is formulated such that administration, such as oral administration or IV administration, of the formulated pharmaceutical composition results in a plasma level of sulfasalazine 60 minutes after such administration that is at least 25%, at least 50%, at least 100%, at least 150%, at least 200%, at least 250% at least 300%, at least 500%, at least 1,000%, at least 2,000% or at least 8,000% higher than the plasma level of sulfasalazine 60 minutes after administration of the same dose level of crystalline sulfasalazine. In certain embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 60 minutes after such administration that is about 25%, about 50%, about 100%, about 150%, about 200%, about 250%, about 300%, about 500%, about 1,000%, about 2,000%, about 8,000%, about 10,000% or about 25,000% higher than the plasma level of sulfasalazine 60 minutes after administration of the same dose level of crystalline sulfasalazine. In yet other embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 60 minutes after such administration that is between about 25% and 25,000%, about 75% and 10,000%, or about 100% and 1,000%, inclusive, higher than the plasma level of sulfasalazine 60 minutes after administration of the same dose level of crystalline sulfasalazine. In certain of the embodiments, the disease or disorder is a neurodegenerative disease or disorder, epilepsy disease or disorder, or brain tumor disease or disorder, as recited above.

In another embodiment, the pharmaceutical composition is formulated such that administration, such as oral administration or IV administration, of the formulated pharmaceutical composition results in a maximum plasma concentration, or exposure (AUC), of sulfasalazine after such administration that is at least 25%, at least 50%, at least 100%, at least 150%, at least 200%, at least 250% at least 300%, at least 500%, at least 1,000%, at least 2,000% or at least 8,000%, 10,000%, 15,000%, 20,000% or about 25,000% higher than the maximum plasma concentration or exposure (AUC) of sulfasalazine after administration of the same dose level of crystalline sulfasalazine. In certain embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a maximum plasma concentration, or exposure (AUC), of sulfasalazine after such administration that is about 25%, about 50%, about 100%, about 150%, about 200%, about 250%, about 300%, about 500%, about 1,000%, about 2,000%, about 8,000%, about 10,000%, 15,000%, 20,000% or about 25,000% higher than the maximum plasma concentration, or exposure (AUC), of sulfasalazine after administration of the same dose level of crystalline sulfasalazine. In yet other embodiments, there are provided methods for treating a disease or disorder in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a maximum plasma concentration, or exposure (AUC), of sulfasalazine after such administration that is between about 25% and 25,000%, about 75% and 10,000%, or about 100% and 1,000%, inclusive, higher than the maximum plasma concentration, or exposure (AUC), of sulfasalazine after administration of the same dose level of crystalline sulfasalazine. In certain of the embodiments, the disease or disorder is a neurodegenerative disease or disorder, epilepsy disease or disorder, or brain tumor disease or disorder as recited above.

In yet other embodiments, there are provided methods for treating ALS or P-MS, and seizure disorders, including Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs) in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 30 minutes after such administration that is between about 25% and 500%, 25% and 25,000%, about 75% and 10,000%, about 75% and 300%, about 100% and 200%, about 100% and 1,000% inclusive; at least 25%, at least 50%, at least 100%, at least 150%, at least 200%, at least 250%, at least 300%, at least 500%, at least 1,000%, at least 2,000%, or at least 6,000% higher than the plasma level of sulfasalazine 30 minutes after administration of the same dose level of crystalline sulfasalazine. In one embodiment, the plasma level is determined by the method of Example 10. In certain of those embodiments, the pharmaceutical composition is in an oral dosage form or in a spray dried dispersion form. In certain embodiments of the above methods, the plasma level is determined based on a rat model.

In yet other embodiments, there are provided methods for treating ALS or P-MS, and seizure disorders, including Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs) in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 60 minutes after such administration that is between about 25% and 500%, 25% and 25,000%, about 75% and 10,000%, about 75% and 300%, about 100% and 200%, about 100% and 1,000% inclusive; at least 25%, at least 50%, at least 100%, at least 150%, at least 200%, at least 250%, at least 300%, at least 500%, at least 1,000%, at least 2,000%, or at least 6,000% higher than the plasma level of sulfasalazine 60 minutes after administration of the same dose level of crystalline sulfasalazine. In one embodiment, the plasma level is determined by the method of Example 10. In certain of those embodiments, the pharmaceutical composition is in an oral dosage form or in a spray dried dispersion form. In certain embodiments of the above methods, the plasma level is determined based on a rat model.

### Formulations of the Invention:

In some embodiments, there is provided a pharmaceutical composition comprising sulfasalazine, an ABCG2 inhibitor and a pharmaceutically acceptable excipient, wherein the pharmaceutical composition has been formulated such that the in vitro solubility of the sulfasalazine is between about 500 μg/ml and 11,500 μg/ml, inclusive; or is between about 500 μg/ml and 7,500 μg/ml, 500 μg/ml and 5,500 μg/ml, 500 μg/ml and about 2500 μg/ml, between about 2300 μg/ml and 11,500 μg/ml, inclusive; or at least 500 μg/ml, 1200 μg/ml or 2300 μg/ml. In one aspect, the solubility is determined at a pH of 5.5 determined as in Example 9. In another aspect, the “in vitro solubility” of sulfasalazine will be considered to be the Cₘₐₓ IB at 90 minutes as shown in Example 9 and Table 9.

In some embodiments, there is provided a pharmaceutical composition comprising sulfasalazine, an ABCG2 inhibitor and a pharmaceutically acceptable excipient, such as in an oral dosage, wherein the pharmaceutical composition has been formulated such that the in vitro solubility of the sulfasalazine at a pH of 5.5 is at least 2 times higher, at least 5 times or at least 8.8 times; or between about 2 times and about 44 times higher than the in vitro solubility of crystalline sulfasalazine in aqueous solution at a pH of 5.5 by AUC analysis. In one aspect, the in vitro solubility is determined as in Example 9.

In some embodiments, there is provided a pharmaceutical composition comprising sulfasalazine and an ABCG2 inhibitor, wherein the pharmaceutical composition has been formulated such that oral administration of such formulated pharmaceutical composition results in a plasma level of sulfasalazine 30 minutes after such administration that is between about 25% and 25,000%, between about 25% and 500%, between about between about 75% and 300%, between about 75% and about 10,000%, or between about 300% and 500%, between about 300% and 1,000%, inclusive; or at least 25%, at least 50%, at least 100%, at least 150%, at least 200%, at least 250%, at least 300%, at least 500%, at least 1,000%, at least 2,000% or at least 6,000% higher than the plasma level of sulfasalazine 30 minutes after administration of the same dose level of crystalline sulfasalazine. In one aspect, the level is determined as in Example 10. In certain of the above embodiments, the sulfasalazine is in an essentially amorphous form.

In other embodiments, there is provided a pharmaceutical composition comprising sulfasalazine and an ABCG2 inhibitor, wherein the pharmaceutical composition has been formulated such that oral administration of such formulated pharmaceutical composition results in a plasma level of sulfasalazine 60 minutes after such administration that is between about 25% and 25,000%, between about 25% and 500%, between about between about 75% and 300%, between about 75% and about 10,000%, or between about 300% and 500%, between about 300% and 1,000%, inclusive; or at least 25%, at least 50%, at least 100%, at least 150%, at least 200%, at least 250%, at least 300%, at least 500%, at least 1,000%, at least 2,000% or at least 6,000% higher than the plasma level of sulfasalazine 60 minutes after administration of the same dose level of crystalline sulfasalazine. In one aspect, the level is determined as in Example 10. In certain of the above embodiments, the sulfasalazine is in an essentially amorphous form.

In one aspect of the above compositions, the ABCG2 inhibitors is selected from the group consisting of Pluronic P85, Tween 20, E-TPGS (TPGS), Pluronic 85, Brij 30, Pluronic L81, Tween 80 and PEO-PPO, or mixtures thereof. In another aspect, the ABCG2 inhibitor is TPGS or Tween 20, or a mixture thereof. In another aspect, the ABCG2 inhibitor is TPGS.

In certain embodiments of the compositions and methods, the composition comprises amorphous or essentially amorphous sulfasalazine, an ABCG2 inhibitor, and optionally a pharmaceutically acceptable polymer. In certain of those embodiments, the pharmaceutical compositions are in the form of a solid dispersion a pharmaceutically acceptable polymer. In certain embodiments, the pharmaceutically acceptable polymer may be selected from polyvinylpyrrolidone (PVP, including PVP VA64, homo- and copolymers of polyvinylpyrrolidone and homopolymers or copolymers of N-vinylpyrrolidone); crospovidone; polyoxyethylene-polyoxypropylene copolymers (also known as poloxamers); cellulose derivatives (including hydroxypropyl methyl cellulose acetate succinate (HPMCAS), hydroxypropyl methyl cellulose phthalate (HPMCP), hydroxypropyl methylcellulose (HPMC), cellulose acetate phthalate (CAP), cellulose acetate trimellitate (CAT), hydroxypropyl methyl cellulose acetate phthalate, hydroxypropyl methyl cellulose acetate trimellitate, cellulose acetate succinate, methylcellulose acetate succinate, carboxymethyl ethyl cellulose (CMEC), hydroxypropyl methyl cellulose, hydroxypropyl methyl cellulose acetate, hydroxyethylcellulose); dextran; cyclodextrins; homo- and copolymers of vinyllactam, and mixtures thereof; gelatins; hypromellose phthalate; sugars; polyhydric alcohols; polyethylene glycol (PEG); polyethylene oxides; polyoxyethylene derivatives; polyvinyl alcohol; propylene glycol derivatives and the like; SLS; Tween; EUDRAGIT (a methacrylic acid and methyl methacrylate copolymer); and combinations thereof. The polymer may be water soluble or water insoluble. In certain embodiments, the ratio of the sulfasalazine to polymer in the composition is about 5:95 wt/wt to 50:50 wt/wt. In certain embodiments, the wt/wt ratio of the ABCG2 inhibitor to sulfasalazine (ABCG2:sulfasalazine) in the composition may be about 9:1, 4:1, 2:1, 1:1, 1:2, 1:3, 1:4, 1:5, 1:10, 1:20, 1:50, 1:100 or about 1:200; or may be about 1:20 wt/wt.

In certain embodiments, there is provided pharmaceutical compositions comprising sulfasalazine, an ABCG2 inhibitor and optionally PVP VA64 or HMPCAS, wherein when present, the wt/wt ratio of the sulfasalazine to PVP VA64 or HMPCAS in the composition is about 20:80 to 30:70, about 40:60 to 60:40, about 50:50, or about 25:75 wt/wt; where the sulfasalazine dispersed in the polymer is in a crystalline or an amorphous form.

In certain embodiments of the methods, the pharmaceutical composition is formulated such that the in vitro solubility of the sulfasalazine is between about 500 μg/ml and 7,500 μg/ml, about 500 μg/ml and 2500 μg/ml, about 2300 μg/ml and 5,500 μg/ml, 2300 μg/ml and 7,500 μg/ml; about 2300 μg/ml and 11,500 μg/ml, about 11,500 μg/ml, inclusive; at least 500 μg/ml, at least 1200 μg/ml or at least 2300 μg/ml, or higher at a pH of 5.5. In one aspect, the solubility is determined as in Example 9.

In other embodiments, there is provided a spray dried dispersion composition comprising sulfasalazine, an ABCG2 inhibitor and a PVP VA64 or HPMCAS polymer. In one aspect, the wt/wt ratio of the sulfasalazine to PVP VA64 or to HPMCAS, in the composition is about 20:80 to 50:50 or about 25:75 wt/wt. In another aspect of the above, the spray dried dispersion composition is formulated such that the in vitro solubility of the sulfasalazine is at least 500 μg/ml, at least 1200 μg/ml or at least 2300 μg/ml, or higher at a pH of 5.5. In another aspect of the above, the spray dried dispersion composition is formulated such that the in vitro solubility of the sulfasalazine is between about 500 μg/ml and 11,500 μg/ml, about 500 μg/ml and 7,500 μg/ml, about 500 μg/ml and 5,500 μg/ml, about 500 μg/ml and 2500 μg/ml, about 2300 μg/ml and 11,500 μg/ml, inclusive; about 500 μg/ml, 1200 μg/ml or 2300 μg/ml, or higher at a pH of 5.5. In one aspect, the solubility is determined as in Example 9.

In certain embodiments of the above compositions and methods, the composition comprises PVP VA64 or HPMCAS. In certain embodiments, the wt/wt ratio of the sulfasalazine to PVP VA64 or HPMCAS in the composition is about 20:80 to 30:70 or is about 25:75 wt/wt. In certain embodiments, the wt/wt ratio of the sulfasalazine to PVP VA64 in the composition is about 40:60 to about 60:40 or is about 50:50 wt/wt. In certain embodiments of the compositions and methods, the sulfasalazine is in an amorphous form or an essentially amorphous form.

In one aspect where the formulation is in a solid dose formulation, such as a tablet or capsule, the formulation may comprise a polymer such as PVP VA64 or HPMCAS. In another aspect where the formulation is a liquid formulation, such as a solution, a gel or a suspension, then a polymer such as PVP VA64 or HPMCAS may be absent.

Treating Neurodegenerative Diseases and Disorders with the Formulations of the Invention:

In certain embodiments, the disclosed formulations can be used in the treatment of neurodegenerative diseases and disorders as well as certain other diseases and disorders. For example, the various formulations and compositions comprising sulfasalazine described in this application can be used in the treatment of seizure disorders, P-MS, ALS, neuropathic pain and other neurodegenerative diseases and disorders.

In certain embodiments, the application provides methods for treating neuropathic pain, such as neuropathic pain results from painful diabetic neuropathy, or neuropathic pain manifests as dysesthesia, or neuropathic pain manifests as allodynia; rheumatoid arthritis or ankylosing spondylitis, in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that the in vitro solubility of the sulfasalazine is between about 500 μg/ml and 11,500 μg/ml, about 500 μg/ml and 7,500 μg/ml, 500 μg/ml and 5,500 μg/ml, about 500 μg/ml and 2500 μg/ml, about 2300 μg/ml and 11,500 μg/ml, inclusive; at least 500 μg/ml, at least 1200 μg/ml or at least 2300 μg/ml. In one aspect, the solubility is determined at a pH of 5.5 determined as in Example 9. In certain embodiments, the application provides methods for treating neuropathic pain in a patient comprising orally administering to the patient a pharmaceutical composition comprising a therapeutically effective amount of sulfasalazine and a pharmaceutically acceptable excipient, wherein the pharmaceutical composition is formulated such that the in vitro solubility of the sulfasalazine is about 500 μg/ml, about 1200 μg/ml or about 2300 μg/ml. In one variation of the method, the solubility is at a pH of 5.5 as determined in Example 9.

In certain embodiments, methods are provided for treating neuropathic pain, such as neuropathic pain results from painful diabetic neuropathy, or neuropathic pain manifests as dysesthesia, or neuropathic pain manifests as allodynia; rheumatoid arthritis or ankylosing spondylitis in a patient, comprising orally administering to the patient a pharmaceutical composition comprising a therapeutically effective amount of sulfasalazine and a pharmaceutically acceptable excipient, wherein the pharmaceutical composition is formulated such that the in vitro solubility of the sulfasalazine at a pH of 5.5 is at least between about 2 times and 44 times, about 2 times and 8.8 times, or between about 8.8 times and 44 times, inclusive; or at least 2 times, at least 5 times, or at least 8.8 times higher than the in vitro solubility of crystalline sulfasalazine at a pH of 5.5 by AUC analysis. In one aspect, the solubility is as determined in Example 9.

In other embodiments, there are provided methods for treating a neurodegenerative disease or disorder in a patient comprising orally administering to the patient a pharmaceutical composition comprising sulfasalazine, an ABCG2 inhibitor and optionally, PVP VA64 or HPMCAS wherein, when present, the wt/wt ratio of the sulfasalazine to PVP VA64 or HPMCAS in the composition is about 20:80 to 50:50. In certain embodiments, the sulfasalazine is dispersed in an essentially amorphous form. In certain embodiments, the sulfasalazine is dispersed in the polymer is in an amorphous form. In certain embodiments, the ratio of sulfasalazine to PVP VA64 or HPMCAS is about 25:75 wt/wt. In certain embodiments, the neurodegenerative disease is selected from Parkinson's disease, Alzheimer's disease, epilepsy, traumatic brain injury, Huntington's disease, ischemic stroke, Rett Syndrome, Frontotemporal Dementia, HIV-associated Dementia and Alexander disease.

In certain embodiments, methods are provided for lowering excessive levels of glutamate in a patient with a neurodegenerative disease comprising orally administering to the patient a pharmaceutical composition comprising a therapeutically effective amount of sulfasalazine, an ABCG2 inhibitor and optionally, a pyrrolidone polymer, wherein when present, the wt/wt ratio of the sulfasalazine to the pyrrolidone polymer in the composition is about 20:80 to 30:70 wt/wt and wherein the sulfasalazine dispersed in the polymer is in an essentially amorphous form. In certain embodiments, the sulfasalazine is dispersed in the polymer is in an amorphous form. In certain embodiments, the ratio of sulfasalazine to pyrrolidone polymer is about 25:75 wt/wt.

In one variation of the compositions or formulations comprising sulfasalazine of the present application, the sulfasalazine is prepared or formulated as described herein, where the sulfasalazine used as a starting material for preparing the composition or the formulation is crystalline sufasalazine.

In one aspect of each of the above embodiments, the neurodegenerative disease or disorder is selected from the group consisting of epilepsy, stroke or traumatic brain injury. In another aspect, the neurodegenerative disease or disorder is Parkinson's disease (PD), Alzheimer's disease (AD) or Huntington's. In another aspect of each of the above embodiments, the neurodegenerative disease or disorder is progressive MS (P-MS), is amyotrophic lateral sclerosis (ALS), or is neuropathic pain. In another variation, the disease or disorder is selected from the group consisting of Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect of the method, the seizures are a symptom of a disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Rasmussen's Syndrome, Hypothalamic Hamartoma, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect, there is provided a method for the treatment of a brain tumor selected from the group consisting of astrocytoma, glioma, glioblastoma, and long-term epilepsy associated tumors (LEATs) selected from ganglioglioma, oligodendroglioma and dysembryoplastic neuroepithelial tumors (DNETs), wherein the method comprises the administration of an effective amount of the above composition to a patient in need thereof. In another aspect of each of the above embodiments, the sulfasalazine is amorphous sulfasalazine.

As disclosed herein, the addition of an ABCG2 inhibitor at a concentration of about 5%, 10%, 15%, 20%, 25%, 30%, 40%, 50%, 60%, 70%, 80%, 90% or about 95% or more, to a sulfasalazine composition, such as a spray-dried dispersion (SDD) comprising sulfasalazine and a polymer, provides at least a 25%, at least 50%, at least 100%, at least 150%, at least 200%, at least 250%, at least 300%, at least 500%, at least 1000%, at least 2000%, at least 6,000%, at least 8,000%, at least 10,000%, at least 12,000%, at least 15,000%, at least 20,000%, at least 25,000% or at least 28,000% increase in the bioavailability of sulfasalazine when compared to the RLD. In some embodiments, the SSD is sulfasalazine and a polymer, such as PVP VA64 or HPMCAS. In another embodiment, the ratio of the sulfasalazine to the ABCG2 inhibitor is from about 1:9 to 200:1 wt/wt. In another embodiment, the ratio of the sulfasalazine to TPGS as defined herein, is from about 1:9 to 200:1 wt/wt. In some variations, the ratio of the sulfasalazine to PVP VA64 or HPMCAS in the pharmaceutical composition is about 20:80 wt/wt to 50:50 wt/wt, or about 25:75 wt/wt. In one variation, the ratio of the sulfasalazine to the ABCG2 inhibitor is from about 1:5, 1:3, 1:2, 1:1, 10:1, 20:1, 30:1, 40:1, 50:1, 60:1, 70:1, 80:1, 90:1, 100:1; 125:1, 150:1, 175:1 or 200:1 wt/wt. In another variation, the ratio of the sulfasalazine to TPGS is from about 1:5, 1:3, 1:2, 1:1, 10:1, 20:1, 30:1, 40:1, 50:1, 60:1, 70:1, 80:1, 90:1, 100:1; 125:1, 150:1, 175:1 or 200:1 wt/wt. In one variation, the ABCG2 efflux inhibitors is selected from the group consisting of Pluronic P85, Tween 20, E-TPGS (TPGS, as defined herein), Pluronic 85, Brij 30, Pluronic L81, Tween 80 and PEO-PPO, or mixtures thereof. In a particular variation, the SSD is 25% sulfasalazine: 75% PVP-VA64, 30% sulfasalazine: 70% PVP-VA64; 35% sulfasalazine: 65% PVP-VA64, 40% sulfasalazine: 60% PVP-VA64; 50% sulfasalazine: 50% PVP-VA64, 60% sulfasalazine: 40% PVP-VA64 or 70% sulfasalazine: 30% PVP-VA64.

Also disclosed herein are method for the treatment of a patient suffering from seizures, the method comprising the administration of the above composition to a patient in need thereof. In another variation, the disclosed method may be used for the treatment of a disease or disorder selected from the group consisting of epilepsy, stroke or traumatic brain injury. In another aspect, the neurodegenerative disease or disorder is Parkinson's disease (PD), Alzheimer's disease (AD) or Huntington's. In another aspect of each of the above embodiments, the neurodegenerative disease or disorder is progressive MS (P-MS), is amyotrophic lateral sclerosis (ALS), or is neuropathic pain. In another aspect of the method, the seizures are symptoms of a disease or disorder is selected from the group consisting of Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect of the method, the seizures are a symptom of a disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Rasmussen's Syndrome, Hypothalamic Hamartoma, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect, there is provided a method for the treatment of a brain tumor selected from the group consisting of astrocytoma, glioma, glioblastoma, and long-term epilepsy associated tumors (LEATs) selected from ganglioglioma, oligodendroglioma and dysembryoplastic neuroepithelial tumors (DNETs), wherein the method comprises the administration of an effective amount of the above composition to a patient in need thereof. Combination Treatment Methods:

In certain aspects of the invention, a patient with ALS is also administered (or co-administered with) riluzole in addition to a pharmaceutical composition of the invention. In certain of these embodiments, the riluzole is administered to the patient concurrently with the pharmaceutical composition; or is administered to the patient at different times than the pharmaceutical composition.

In certain aspects of the disclosed methods, a patient with P-MS is administered (or co-administered with) Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast in addition to a pharmaceutical composition of the invention. In certain of these embodiments, the Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast is administered to the patient concurrently with the pharmaceutical composition. In certain embodiments, the Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast is administered to the patient at different times than the pharmaceutical composition.

In certain aspects of the disclosed methods, a patient with seizure disorders is also administered (or co-administered with) Acetazolamide, Carbamazepine, Clobazam, Clonazepam, Eslicarbazepine acetate, Ethosuximide, Gabapentin, Lacosamide, Lamotrigine, Levetiracetam, Nitrazepam, Oxcarbazepine, Perampanel, Piracetam, Phenobarbital, Phenytoin, Pregabalin, Primidone, Retigabine, Rufinamide, Sodium valproate, Stiripentol, Tiagabine, Topiramate, Vigabatrin, Zonisamide in addition to a pharmaceutical composition of the invention. In certain embodiments, the Acetazolamide, Carbamazepine, Clobazam, Clonazepam, Eslicarbazepine acetate, Ethosuximide, Gabapentin, Lacosamide, Lamotrigine, Levetiracetam, Nitrazepam, Oxcarbazepine, Perampanel, Piracetam, Phenobarbital, Phenytoin, Pregabalin, Primidone, Retigabine, Rufinamide, Sodium valproate, Stiripentol, Tiagabine, Topiramate, Vigabatrin, Zonisamideis administered to the patient concurrently with the pharmaceutical composition. In certain embodiments, the Acetazolamide, Carbamazepine, Clobazam, Clonazepam, Eslicarbazepine acetate, Ethosuximide, Gabapentin, Lacosamide, Lamotrigine, Levetiracetam, Nitrazepam, Oxcarbazepine, Perampanel, Piracetam, Phenobarbital, Phenytoin, Pregabalin, Primidone, Retigabine, Rufinamide, Sodium valproate, Stiripentol, Tiagabine, Topiramate, Vigabatrin, Zonisamideis administered to the patient at the same or at different times than the pharmaceutical composition. Dosing Regimens for the Treatment of P-MS and Other Neurological Diseases, including epilepsies and brain tumors, as described herein:

In certain embodiments, the present invention provides methods of treating P-MS in patients by administering a therapeutically effective amount of a system x_{c}⁻ inhibitor to such patients. In certain embodiments, the system x_{c}⁻ inhibitor is sulfasalazine. Previous work has tested use of sulfasalazine for treatment of multiple sclerosis (both RR-MS and P-MS) in humans, e.g. Noseworthy et al, Neurology 15: 1342-1352 (1998). Patients were treated with 2 grams of sulfasalazine per day, the typical maintenance dose used for non-CNS diseases, such as rheumatoid arthritis, e.g. Khan et al, Gut 21:232-240 (1980). Sulfasalazine did not slow disease progression in the RR-MS sub-group. In the P-MS subgroup, patients treated with sulfasalazine had a statistically significant reduction in their accumulation of disability, which the authors attributed to a “real treatment effect.” See id. at p. 1346. However, to our knowledge, no further clinical trials of sulfasalazine for the treatment of either P-MS or RR-MS have been performed. Other previous work demonstrated that a 2 g oral dose of sulfasalazine administered to humans produced plasma levels above 10 μg/ml that were maintained for only approximately 7 hours in people with the ABCG2 genotype (421C/C) (see Yamasaki et al, Clin. Pharmac. Therap. 84: 95-103 (2007)), which is the predominant ABCG2 genotype in European Caucasian and African American populations (77%-90%) see, e.g., de Jong et al, Clin. Cancer Res. 10:5889-5894 (2004). Prior work had also shown that the anti-epileptic effect of sulfasalazine administered to a mouse model at a dose of approximately 260-320 mg/kg intraperitoneal (“IP”) is lost between two to three hours after administration (see Buckingham et al, Nat Med. 17:1269-1274 (2011)). As experiments described herein indicate that the plasma level of sulfasalazine in a mouse administered a 200 mg/kg dose IP of sulfasalazine (approximately 30-60% lower dose than the Buckingham study) is about 6 μg/ml at two hours after administration (see Example 4), the inventors determined that a plasma level of sulfasalazine of at least approximately 8-10 μg/ml (adjusted for dose differences) is needed is needed for a therapeutic effect by sulfasalazine on the system x_{c}⁻ in the CNS compartment. Based on this, the inventors hypothesize that the P-MS patients treated with sulfasalazine in the Noseworthy study were under-dosed. Thus, the invention also provides methods of treating P-MS with sulfasalazine using improved dosing regimens and formulations.

In another variation, the disclosed method provides for the treatment of seizures, wherein, the seizures are symptoms of a disease or disorder is selected from the group consisting of Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect of the method, the seizures are a symptom of a disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Rasmussen's Syndrome, Hypothalamic Hamartoma, Tuberous Sclerosis Complex, Focal Cortical Dysplasia and epileptic encephalopathies and seizures related to brain tumors, including but not limited to astrocytoma, glioma, glioblastoma and long-term epilepsy associated tumors (LEATs) for example ganglioglioma, oligodendroglioma, and dysembryoplastic neuroepithelial tumors (DNETs). In another aspect, there is provided a method for the treatment of a brain tumor selected from the group consisting of astrocytoma, glioma, glioblastoma, and long-term epilepsy associated tumors (LEATs) selected from ganglioglioma, oligodendroglioma and dysembryoplastic neuroepithelial tumors (DNETs), wherein the method comprises the administration of an effective amount of the above composition to a patient in need thereof.

In certain embodiments, the present invention provides methods for treating P-MS in a patient comprising administering to the patient a pharmaceutical composition comprising sulfasalazine and an ABCG2 inhibitor, wherein the sulfasalazine is dosed at levels and/or frequencies sufficient to produce improved therapeutic effects. In certain embodiments, methods are provided for treating a patient with P-MS comprising orally administering to the patient a pharmaceutical composition comprising sulfasalazine, an ABCG2 inhibitor and a pharmaceutically acceptable excipient, wherein the dose of the pharmaceutical composition is sufficient to maintain a plasma level of sulfasalazine in the patient effective for treating P-MS for at least 14 total hours a day. In certain embodiments, a plasma level of sulfasalazine in the patient effective for treating P-MS is maintained for between 21 and 24, inclusive, total hours a day; or is maintained for 24 hours a day.

In certain embodiments, methods are provided for treating a patient with P-MS comprising orally administering to the patient a pharmaceutical composition comprising sulfasalazine and a pharmaceutically acceptable excipient, wherein the dose of the pharmaceutical composition is sufficient to maintain a plasma level of sulfasalazine of at least 8 μg/ml for at least 14 total hours a day. In certain embodiments, a plasma level of sulfasalazine of at least 8 μg/ml is maintained for between 21 and 24, inclusive, total hours a day. In certain embodiments, a plasma level of sulfasalazine of at least 8 μg/ml is maintained for 24 hours a day. In certain embodiments, the dose of the pharmaceutical composition is sufficient to maintain a plasma level of sulfasalazine of between about 8 μg/ml and 30 μg/ml, inclusive, or between about 8 μg/ml and 16 μg/ml, inclusive, or between about 10 μg/ml and 16 μg/ml, inclusive, for the given amount of time; or for the entire dosing interval. For the purposes of this application, the condition “for the entire dosing interval” will be considered to be met if the level of the sulfasalazine is at or above the designated level at the end of the dosing interval (but before any next administration of the sulfasalazine). In certain embodiments, the dose of the pharmaceutical composition is sufficient to produce a plasma level of sulfasalazine in the patient of between about 8 μg/ml and 30 μg/ml, between about 10 μg/ml and 30 μg/ml, between about 8 μg/ml and 16 μg/ml or between about 8 μg/ml and 12 μg/ml, inclusive; at least 10 μg/ml, or 16 μg/ml for the entire dosing interval.

One way to increase plasma levels of sulfasalazine is to administer higher daily doses of the standard formulation of sulfasalazine to patients. Previous work has demonstrated that, in humans, plasma levels of sulfasalazine are proportional to the oral dose, e.g. Khan et al, Gut 21:232-240 (1980). In certain embodiments, the present invention provides methods for treating a patient with P-MS comprising orally administering to the patient a pharmaceutical composition comprising sulfasalazine, an ABCG2 inhibitor and a pharmaceutically acceptable excipient, wherein the pharmaceutical composition is a standard formulation of sulfasalazine and the total daily dose of sulfasalazine is between about 10 mg and 8 grams, between about 2.5 grams and 8 grams, between about 3 mg and 5 grams, or between about 10 mg and 5 grams, inclusive; or about 10 mg, 100 mg, 250 mg, 500 mg, 750 mg, 1 gram, 2 grams, 3 grams, 4 grams, or 5 grams.

In certain embodiments, there is provided methods for treating a patient with P-MS comprising orally administering to the patient a pharmaceutical composition comprising sulfasalazine, an ABCG2 inhibitor and a pharmaceutically acceptable excipient, wherein (a) the pharmaceutical composition is a standard formulation of sulfasalazine, (b) the dose at each dosing time point is not more than about 4 grams of sulfasalazine, (c) there are at least two dosing time points in a day, and (d) the total daily dose is between about 10 mg and 8 grams, about 2.5 grams and 8 grams, about 2.5 grams and 6 grams, about 10 mg and 6 grams, about 3 grams and 5 grams, about 10 mg and 5 grams, inclusive; or about 10 mg, 100 mg, 250 mg, 500 mg, 750 mg, 1 gram, 2 grams, 3 grams, 4 grams, or 5 grams, and is administered once a day.

Treatment of Diseases and Disorders Other than Neurodegenerative Diseases and Disorders:

In other embodiments, there is provided a method for treating diseases where sulfasalazine is currently used clinically and is believed to act systemically, including rheumatoid arthritis and ankylosing spondylitis, wherein such method comprises administering a composition of the invention comprising sulfasalazine in which the solubility and/or the bioavailability of the sulfasalazine is increased. In rheumatoid arthritis, the typical maintenance dose of sulfasalazine is 2 g/day. Higher doses have been shown to result in greater efficacy, but unfortunately, the higher doses of sulfasalazine also result in a higher incidence of toxicity, e.g. Khan et al, Gut 21:232-240 (1980). By increasing the solubility and/or the bioavailability of the sulfasalazine, the present invention provides a method of increasing the therapeutic dose of sulfasalazine without an increase in the toxicity.

In certain embodiments, there are provided methods for treating rheumatoid arthritis and/or ankylosing spondylitis in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 30 minutes after such administration that is at least 25%, at least 50%, at least 100%, at least 150%, at least 200%, at least 250% at least 300%, at least 500%, at least 1,000%, at least 2,000%, or at least 6,000% higher than the plasma level of sulfasalazine 30 minutes after administration of the same dose level of crystalline sulfasalazine. In one aspect of the method, the plasma level is as determined by the method of Example 10.

In certain embodiments, there are provided methods for treating rheumatoid arthritis and/or ankylosing spondylitis in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a plasma level of sulfasalazine 60 minutes after such administration that is at least 25%, at least 50%, at least 100%, at least 150%, at least 200%, at least 250% at least 300%, at least 500%, at least 1,000%, at least 2,000%, or at least 6,000% higher than the plasma level of sulfasalazine 60 minutes after administration of the same dose level of crystalline sulfasalazine. In one aspect of the method, the plasma level is as determined by the method of Example 10.

In certain embodiments, there are provided methods for treating rheumatoid arthritis and/or ankylosing spondylitis in a patient comprising orally administering to the patient a pharmaceutical composition comprising a therapeutically effective amount of sulfasalazine, an ABCG2 inhibitor and a pharmaceutically acceptable excipient, wherein the pharmaceutical composition is formulated such that oral administration of the formulated pharmaceutical composition results in a maximum plasma concentration, or exposure (AUC), of sulfasalazine after such administration that is at least 25%, at least 50%, at least 100%, at least 150%, at least 200%, at least 250% at least 300%, at least 500%, at least 1,000%, at least 2,000%, or at least 6,000% higher than the maximum plasma concentration, or exposure (AUC), of sulfasalazine after administration of the same dose level of crystalline sulfasalazine. In one aspect of the method, the plasma level is as determined by the method of Example 10.

In certain embodiments, the pharmaceutical composition is formulated such that oral administration of the formulated pharmaceutical composition results in a plasma level of sulfasalazine 30 minutes after such administration that is between about 25% and 25,000%, between about 75% and 10,000% or between about 100% and 1,000%, inclusive, higher than the plasma level of sulfasalazine 30 minutes after administration of the same dose level of crystalline sulfasalazine. In one aspect, the plasma level is as determined by the method of Example 10.

In certain embodiments, the pharmaceutical composition is formulated such that oral administration of the formulated pharmaceutical composition results in a plasma level of sulfasalazine 60 minutes after such administration that is between about 25% and 25,000%, between about 75% and 10,000% or between about 100% and 1,000%, inclusive, higher than the plasma level of sulfasalazine 60 minutes after administration of the same dose level of crystalline sulfasalazine. In one aspect, the plasma level is as determined by the method of Example 10.

In certain embodiments, the pharmaceutical composition is formulated such that oral administration of the formulated pharmaceutical composition results in a maximum plasma concentration, or exposure (AUC), of sulfasalazine after such administration that is between about 25% and 25,000%, between about 75% and 10,000% or between about 100% and 1,000%, inclusive, higher than the maximum plasma concentration, or exposure (AUC), of sulfasalazine after administration of the same dose level of crystalline sulfasalazine. In one aspect, the plasma level is as determined by the method of Example 10.

In certain embodiments, methods are provided for treating a patient with rheumatoid arthritis and/or ankylosing spondylitis comprising orally administering to the patient a pharmaceutical composition comprising a therapeutically effective amount of sulfasalazine, an ABCG2 inhibitor and a pharmaceutically acceptable excipient, wherein the pharmaceutical composition optionally comprises PVP VA 64 or HPMCAS, and when present, the ratio of the sulfasalazine to PVP VA64 or HPMCAS in the pharmaceutical composition is about 20:80 to 50:50 wt/wt. In certain of those embodiments, the ratio of sulfasalazine to PVP VA64 or HPMCAS is about 25:75 wt/wt. In certain embodiments, the sulfasalazine dispersed in the polymer is in an essentially amorphous form. In one variation of the method, the composition excludes PVP VA64 or HPMCAS.

Other System x_{c}⁻ Inhibitors:

In some embodiments, methods for treating a patient with a neurodegenerative disease or disorder comprising administering to the patient an effective amount of an inhibitor of system x_{c}⁻ other than sulfasalazine are provided. In certain embodiments, the system x_{c}⁻ inhibitor is selected from (S)-4-carboxyphenylglycine, 2-hydroxy-5-((4-(N-pyridin-2-ylsulfamoyl)phenyl)ethynyl)benzoic acid, aminoadipate (AAA), 4-(1-(2-(3,5-bis(trifluoromethyl)phenyl)hydrazono)ethyl)-5-(4 (trifluoromethyl)benzyl)isoxazole-3-carboxylic acid, 5-benzyl-4-(1-(2-(3,5-bis(trifluoro-methyl)phenyl)hydrazono)ethyl)isoxazole-3-carboxylic acid and 2-hydroxy-5-[2-[4-[(3-methylpyridin-2-yl)sulfamoyl]phenyl]ethynyl] benzoic acid.

The following embodiments, aspects and variations thereof are exemplary and illustrative are not intended to be limiting in scope.

### DESCRIPTION OF THE FIGURES

FIG. 1 shows a representative Kaplan-Meier absolute survival curves in SOD1^{G93A} mice (herein after “SOD1 mice”). The vehicle-treated (CTRL) and sulfasalazine-treated (DRUG) cohorts are plotted in gray and black, respectively.

FIG. 2 is representative of histograms showing the distribution of lifespan in vehicle and sulfasalazine treated mice after onset of definitive neurological disease.

FIG. 3 shows a representative graph of the percent change in lifespan after onset of definitive neurological disease in SOD1 mice treated with riluzole, ibuprofen, MR1 and sulfasalazine.

FIG. 4 shows representative samples from Day 100 mice stained for xCT protein expression (brown).

FIG. 5 is representative of an area fraction analysis of xCT expression in the ventral horn of the cervical and lumbar regions of the spinal cord in day 85 and day 100 mice. The symbol ‘*’ indicates the indicated measurement between groups reached a statistical significance of p<0.05.

FIG. 6 shows a representative graph of an area fraction analysis comparing xCT expression in day 85 and day 100 mice. The y-axis quantifies the xCT expression in the ventral horn of the combined cervical, thoracic and lumbar spinal cord regions in vehicle treated SOD1 mice and wild-type mice. The symbol ‘*’ indicates the indicated measurement between groups reached a statistical significance of p<0.05.

FIG. 7 shows representative images from the ventral horn of the spinal cord from Day 85 mice stained for microglial activation using anti-F4/80 antibody. Activated microglia are stained brown.

FIG. 8 shows representative samples from Day 100 mice stained for astrocyte activation using anti-GFAP antibody. Activated astrocytes are stained brown.

FIG. 9 shows area fraction quantitation of the activated astrocytes and microglial cells in the ventral horn from the cervical and lumbar regions in day 85 mice. Images were analyzed in a blinded fashion and mean area fraction occupied by stain was tabulated. The symbols ‘*’ and ‘**’ indicate the indicated measurement between groups reached a statistical significance of p<0.05 and p<0.01, respectively.

FIG. 10 shows area fraction quantitation of the activated astrocytes and microglial cells in the ventral horn from the cervical and lumbar regions in day 100 mice. The symbols ‘*’, ‘**’ and ‘***’ indicate the measurement between groups reached a statistical significance of p<0.05, p<0.01 and p<0.001, respectively.

FIG. 11 shows a representative graph of the concentrations of sulfasalazine in the spinal cord versus plasma in a scatter plot format. The trendline is shown as a dashed line. The bounds of minimal and significant inhibition are shown as dotted lines.

FIG. 12 is a graphical representation of the in vitro solubility of sulfasalazine as a function of pH.

FIG. 13 is a representative graph of results from powder X-ray diffraction (PXRD) analysis of various sulfasalazine formulations.

FIG. 14 is a representative graph of results from modulated differential scanning calorimetry (mDSC) analysis of sulfasalazine compositions. The resulting glass-transition temperature (Tg) curve is used to determine the homogeneity of the composition.

FIG. 15 is a representative graph of results measuring solubility of sulfasalazine preparations at gastric buffer (GB) and intestinal buffer (IB) of the sulfasalazine formulations.

FIG. 16 is a representative graph of results showing an increase in oral bioavailability of sulfasalazine in a Sprague-Dawley rats following reformulation.

FIG. 17 graphically shows the data from the Amplex Red Glutamic Acid/Glutamate Oxidase assay that is contained in Table 19. The known glutamate concentrations are shown on the x-axis and the fluorescence detected is shown on the Y-axis.

FIG. 18 graphically shows the data from the Amplex Red Glutamic Acid/Glutamate Oxidase assay that is contained in Table 20. The time in minutes after the astrocyte media was changed to minimal media is shown on the x-axis and the extracellular glutamate detected is shown on the Y-axis.

FIG. 19 is a representative graph of results from powder X-ray diffraction (PXRD) analysis of various sulfasalazine formulations.

FIG. 20 is a representative graph of results measuring solubility of sulfasalazine preparations in a single stage in vitro dissolution test.

FIG. 21 is a representative graph of results measuring solubility of sulfasalazine preparations in a two-stage in vitro dissolution test.

FIG. 22 is a representative graph of results showing an increase in oral bioavailability of sulfasalazine in beagle dogs following reformulation.

FIG. 23 is a representative graph of results showing an increase in oral bioavailability of sulfasalazine in rats following reformulations described in Table 27. The mean plasma concentration of sulfasalazine (ng/ml) is plotted in linear format on the y-axis and time in minutes is plotted on the x-axis.

In addition to the exemplary embodiments, aspects and variations described above, further embodiments, aspects and variations will become apparent by reference to the drawings and figures and by examination of the following descriptions.

### DETAILED DESCRIPTION OF THE INVENTION

### Definitions

Unless specifically noted otherwise herein, the definitions of the terms used are standard definitions used in the art of organic synthesis and pharmaceutical sciences. Exemplary embodiments, aspects and variations are illustrated in the figures and drawings, and it is intended that the embodiments, aspects and variations, and the figures and drawings disclosed herein are to be considered illustrative and not limiting.

As used herein, “neurodegenerative disease or disorder” means diseases of the nervous system that are caused, at least in part, by excessive glutamate signaling. Examples of neurodegenerative diseases where anti-glutamatergics are used clinically include Parkinson's disease (Amantadine and Budipine), Alzheimer's disease (Memantine), neuropathic pain (Topamax, Pregabalin), epilepsy (Carbamazepine, Lamictal, Keppra), and ALS (Rilutek). Anti-glutamatergic agents are also being investigated for use in traumatic brain injury, Huntington's disease, ischemic stroke and multiple sclerosis.

As used herein, “amorphous” refers to a form of a compound (e.g., sulfasalazine) that is non-crystalline, having no or substantially no molecular lattice structure, wherein the three dimensional structure positions of the molecules relative to one another are essentially random. Amorphous can mean either the liquid or solid state. When in a liquid state (e.g. a solution or suspension) a compound will by definition be amorphous. When in the solid state, an amorphous material will have liquid-like short range order and, when examined by X-ray diffraction, will generally produce broad, diffuse scattering and will result in peak intensity sometimes centered on one or more broad bands (known as an amorphous halo). PXRD analysis of a solid amorphous material will provide a 2-theta pattern with one or more broad bands with no distinctive peaks; unlike the patterns of a crystalline solid material. “Essentially amorphous” means that the compound in the material is in at least 80% amorphous form (that is, no more than 20% crystalline compound), which means such material, when in the solid state, may exhibit one or more distinctive peaks in a PXRD analysis.

“Bioavailability” refers the percentage of a dose of a drug that enters the circulation when that dose of the drug is administered orally to a human, rodent or other animal.

“In vitro solubility” in reference to the solubility of sulfasalazine means the Cₘₐₓ IB at 90 minutes value for the solubility of sulfasalazine (for example, as exemplified in Table 9) when measured by the methods, for example, of Example 9.

“Standard formulation of sulfasalazine” refers to formulations of sulfasalazine that are considered to be essentially equivalent to Azulfidine in terms of the bioavailability of the sulfasalazine in the formulation. These formulations may include Azulfidine®, Azulfidine® EN (enteric coated), Salazopyrin®, Salazopyrin® EN (enteric coated), SULFASALAZINE TABLETS (Watson Laboratories), SULFAZINE© (Vintage Pharmaceuticals, Inc.), SazoEn (Wallace Pharmaceuticals Ltd.), Salazopyrin EN (Wallace Pharmaceuticals Ltd.), Salazopyrin (Wallace Pharmaceuticals Ltd.), Sazo EC (Wallace Pharmaceuticals Ltd.), Saaz (IPCA Laboratories Ltd.), Saaz DS (IPCA Laboratories Ltd.), Zemosal (Sun Pharmaceutical Industries Ltd.), Colizine (Synmedic Laboratories), Iwata (Cadila Pharmaceuticals Ltd.), and Salazar EC (Cadila Pharmaceuticals Ltd.).

“Dosing interval” in this application means the period of time between administrations of a composition to a patient. For example, if a drug is administered to a patient every 8 hours, then the dosing interval is the 8 hour period that follows the administration of the drug. The condition “for the entire dosing interval” will be considered to be met if the level of the sulfasalazine is at or above the designated level at the end of the dosing interval (but before any next administration of the sulfasalazine).

“Excipient” is a material used in the compositions of the present application, and may be solid, semisolid or liquid materials which serve as vehicles, carriers or medium for the active compound, such as sulfasalazine. Typical excipients may be found in Remington: The Science and Practice of Pharmacy, A. Gennaro, ed., 20th edition, Lippincott, Williams & Wilkins, Philadelphia, Pa.; Handbook of Pharmaceutical Excipients by Raymond C. Rowe et al. 7th Edition, Pharmaceutical Press, London, UK and The United States Pharmacopeia and National Formulary (USP-NF), Rockville, Md. Excipients may include pharmaceutically acceptable polymers.

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid etc.; or with organic acids such as acetic acid, propionic acid, hexanoic acid, malonic acid, succinic acid, malic acid, citric acid, gluconic acid and salicylic acid.

“PVP VA64” as used herein, means vinylpyrrolidone-vinyl acetate copolymers with the general formula (C₆H₉NO)ₙ×(C₄H₆O₂)ₘ. Sources of PVP VA64 include BASF (Ludwigshafen, Germany) as Kollidon® VA 64 and Shanghai Lite Chemical Technology Co., Ltd. as Copovidone (PVP/VA64).

“Copolyvidone”, “Crospovidone” or “polyvinylpyrrolidone polyvinylacetate” is a polyvinylpyrrolidone polyvinylacetate copolymer.

“Polyvinylpyrrolidone” or “PVP” refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum soluble and Poly(1-vinyl-2-pyrrolidone). The copolymer vinyl acetate vinylpyrrolidone is known to the pharmaceutical industry as Copolyvidon, Copolyvidone and Copolyvidonum.

“Progressive multiple sclerosis” or “P-MS” refers to all the sub-types of Progressive Multiple Sclerosis characterized by chronic accumulation of disability, which are Primary Progressive Multiple Sclerosis (PP-MS), Secondary Progressive Multiple Sclerosis (SP-MS) and Progressive-Relapsing Multiple Sclerosis (PR-MS).

“Therapeutically effective amount” means an amount of sulfasalazine or other active ingredient of the application that elicits any of the treatment effects listed in the specification. As used herein, when a unit dose of an active ingredient in the present application is administered in multiple doses a day, the term “therapeutically effective amount” includes unit doses that are themselves sub-therapeutic, but that cumulatively result in an administered amount that elicits a treatment effect.

“Treating” or “treatment” of a disease as used herein means (a) inhibiting or delaying progression of the disease, (b) reducing the extent of the disease, (c) reducing or preventing recurrence of the disease, and/or (d) curing the disease. Treating or treatment include, but are not limited to, one or more of (1) limiting, inhibiting or reducing the rate of accumulation of disability and/or loss of motor neuron function; (2) delaying the progression of the disease, such as neuropathic pain, neuropathic pain results from painful diabetic neuropathy, or neuropathic pain manifests as dysesthesia, or neuropathic pain manifests as allodynia; rheumatoid arthritis or ankylosing spondylitis; epilepsies and seizure disorders, P-MS or ALS; (3) limiting, inhibiting or reducing neuronal dysfunction and/or muscular atrophy, (4) limiting or arresting its development, (5) relieving the disease, i.e., causing the regression of epilepsies and seizure disorders, P-MS or ALS; (6) reducing or preventing the recurrence of the accumulation of disability and/or the loss of motor neuron function; (7) reducing or preventing the recurrence of neuronal dysfunction and/or muscular atrophy; (8) palliating the symptoms of the disease, (9) increase in survival after onset of epilepsies and seizure disorders, P-MS or ALS; and/or, (10) attenuation of neuroinflammation.

### Therapeutic Compositions:

The invention provides pharmaceutical compositions for use in treating neurodegenerative diseases or disorders. In some embodiments, the pharmaceutical compositions comprising sulfasalazine and an ABCG2 inhibitor are formulated such that the bioavailability of the sulfasalazine in the administered pharmaceutical composition is increased by at least 10%, 20%, 30%, 50%, 75%, 80% or at least 90% more, in comparison to administration of crystalline sulfasalazine or the standard formulation of sulfasalazine. In certain embodiments, the pharmaceutical compositions comprising sulfasalazine and an ABCG2 inhibitor are formulated such that the bioavailability of the sulfasalazine in the administered pharmaceutical composition is increased by at least 100%, at least 150%, at least 200%, at least 250%, at least 300%, at least 500%, at least 1000%, at least 2000%, at least 6,000%, at least 8,000%, at least 10,000%, at least 12,000%, at least 15,000%, at least 20,000%, at least 25,000% or at least 28,000% more, in comparison to administration of crystalline sulfasalazine or the standard formulation of sulfasalazine.

In one aspect, the pharmaceutical compositions of the invention comprise sulfasalazine, an ABCG2 inhibitor and optionally, a polymer, wherein the ratio of sulfasalazine to polymer in the composition is about 1:99 wt/wt to 50:50 wt/wt. In another aspect, the ratio of sulfasalazine to polymer is about 5:95 wt/wt to 45:55 wt/wt, about 10:90 wt/wt to 40:60 wt/wt, about 15:85 wt/wt to 35:65 wt/wt, or about 20:80 wt/wt to 30:70 wt/wt.

Also provided are pharmaceutical compositions comprising pharmaceutically acceptable excipients. Such excipients include, but are not limited to, lactose, mannitol, microcrystalline cellulose, crospovidone, croscarmellose, sodium starch glycolate, magnesium stearate or stearic acid, colloidal silicon dioxide, sodium chloride, sodium citrate, polyvinylpyrrolidinone, gelatin, hydroxyethyl-, hydroxypropyl- or hydroxypropylmethyl cellulose, acacia, polyethylene glycol, and other pharmaceutically acceptable polymers. Pharmaceutically acceptable solid, semi-solid or liquid carriers may be added to enhance or stabilize the composition, or to facilitate preparation of the composition. Liquid carriers include syrup, peanut oil, olive oil, other natural, synthetic, or semisynthetic oils, mixed glycerides, medium-chain fatty acids and/or glycerides, glycerin, saline, alcohols or water. Solid carriers include starch, lactose, microcrystalline cellulose, calcium sulfate dihydrate, talc, pectin, acacia, agar or gelatin. Semi-solid carriers include hydrophilic and lipophilic waxes of natural, synthetic or semi-synthetic origin. The carrier may also include a sustained release material such as hypromellose, ethylcellulose or acrylic polymers, or glyceryl monostearate or glyceryl distearate, alone or with other release controlling polymers or waxes. The amount of solid carrier varies but may be between about 20 mg to about 1 g per dosage unit. The pharmaceutical preparations are made following the conventional techniques of pharmacy, including but not limited to milling, mixing, blending, wet granulation, melt-granulation, dry-granulation, extrusion, calendaring, compressing, and coating when necessary, for tablet forms; or milling, mixing, blending, granulation (by wet, dry or melt granulation techniques), extrusion, calendaring, and filling for hard shell capsule forms. Alternatively, the solid pharmaceutical composition may be sprinkeled on food or dissolved or suspended in drinks. Other standard manufacturing procedures are described in in Remington's Pharmaceutical Science, 14th Ed, pp 1626-1678 (1970), published by Mack Publishing Co, Easton, Pa., or more recent editions of the same reference; The Theory and Practice of Industrial Pharmacy by Lachman (2010). When a liquid carrier is used, the preparation will be in the form of a solution, suspension, emulsion, or any other aqueous or non-liquid. Such a liquid formulation may be administered directly by mouth or filled into a soft gelatin capsule.

In some embodiments, pharmaceutical compositions include a pharmaceutically acceptable, non-toxic composition formed by the incorporation of any of the normally employed excipients, such as, mannitol, lactose, starch, magnesium stearate, sodium saccharine, talcum, cellulose, sodium crosscarmellose, glucose, gelatin, sucrose, magnesium carbonate, and combinations thereof. Such compositions include suspensions, tablets, dispersible tablets, pills, capsules, powders and sustained release formulations.

In addition, the compositions can comprise pharmaceutically acceptable carriers or customary auxiliary substances, such as glidants; wetting agents; emulsifying and suspending agents; preservatives; antioxidants; counterirritants; buffering agents, chelating agents; coating auxiliaries; emulsion stabilizers; film formers; gel formers; odor masking agents; taste corrigents; solvents; solubilizers; neutralizing agents; diffusion enhancers; pigments; surfactants; sweeteners; spreading auxiliaries; stabilizers; tablet auxiliaries, such as binders, fillers, disintegrants, lubricants, glidants or coatings agents; drying agents; thickeners; plasticizers and anti-tacking agents; suppository bases, gel or semi-solid bases.

In some embodiments, the pharmaceutical compositions are administered in oral dosage form. Oral dosage forms that may be used include pills, tablets, chewable tablets, capsules, oral liquids, sustained release formulations, and suspensions. In some embodiments where the composition is a pill or tablet, the composition may contain, along with sulfasalazine and an ABCG2 inhibitor, a diluent such as lactose, sucrose, dicalcium phosphate; a lubricant such as magnesium stearate or the like; and a binder such as starch, gum acacia, gelatin, polyvinylpyrolidine, cellulose and derivatives thereof. In other embodiments, tablet forms of the composition may include one or more of lactose, sucrose, mannitol, corn starch, potato starch, alginic acid, microcrystalline cellulose, acacia, gelatin, guar gum, colloidal silicon dioxide, crosscarmellose sodium, talc, magnesium stearate, calcium stearate, zinc stearate, stearic acid, preservatives, flavoring agents, pharmaceutically acceptable disintegrating agents, moistening agents and pharmacologically compatible carriers; and combinations thereof. In other embodiments, formulations suitable for oral administration can consists of liquid solution or suspensions such as an effective amount of sulfasalazine dissolved or suspended in diluents such as water, saline or non-aqueous vehicles. The diluent may contain suspending agents, thickeners, flocculating agents, buffers and pH adjusting agents, preserving agents, osmotic agents, coloring agents; sachets, lozenges and troches, each containing a predetermined amount of sulfasalazine as solids or granules; powders, suspensions in the above appropriate liquid; and suitable emulsions.

In certain embodiments, pharmaceutical compositions containing a solid dispersion of sulfasalazine, an ABCG2 inhibitor and at least one polymer are provided wherein the sulfasalazine is present in essentially amorphous form. In other embodiments, a method for producing a solid molecular dispersion of amorphous sulfasalazine is provided herein involves solvent spray drying. Other techniques that can be used to prepare solid molecular dispersions of amorphous sulfasalazine include: (1) milling; (2) extrusion; (3) melt processes, including high melt-congeal processes and melt-congeal processes; (4) solvent modified fusion; (5) solvent processes, including spray coating, lyophilization, and solvent evaporation (e.g., rotary evaporation); and (6) non-solvent precipitation.

In one aspect, the pharmaceutical compositions are formulated through spray drying. Other methods for creating spray dried compositions are disclosed in EP1469830, EP1469833, EP1653928, WO 2010/111132, WO 96/09814; WO 97/44013; WO 98/31346; WO 99/66903; WO 00/10541; WO 01/13893, WO 2012/031133, WO 2012/031129, and U.S. Pat. Nos. 6,763,607, 6,973,741, 7,780,988 and 8,343,550. In certain of these embodiments, the volume mean diameter of the spray dried dispersion is less than about 500 micrometers in diameter or less than about 200 micrometers or less than 100 micrometers or less than 50 micrometers or less than 10 micrometers. In certain embodiments, the pharmaceutical compositions of the invention are formulated as nanoparticles. Other approaches for formulating pharmaceutical compositions as nanoparticles include WO 2009/073215, U.S. Pat. Nos. 8,309,129; 8,034,765 and 5,118,528.

Typical loadings of sulfasalazine in the formulation can range from 1 wt % API to 50 wt % in the compositions, or will range from 5 wt % API to 50 wt %, or 10 wt % to 40 wt %. This will depend on several factors, including (1) the nature of the polymers in the composition, and (2) the storage stability of the composition (e.g., its tendency to phase separate). The sulfasalazine prepared and used in the compositions of the present application may be amorphous. In one particular aspect, the PXRD spectrum of the amorphous sulfasalazine shows a 2-theta pattern with a broad band having no distinctive peaks. In another aspect, the sulfasalazine used in the compositions are at least 80% amorphous, 90% amorphous, at least 93% amorphous, at least 95% amorphous, at least 97% amorphous, at least 98% amorphous, at least 99% amorphous, at least 99.5% amorphous or about 100% amorphous. In another aspect, the remaining or the balance of the sulfasalazine used in the compositions are crystalline material, semi-crystalline material or combination of crystalline and semi-crystalline materials as determined by PXRD.

Also included in the above embodiments, aspects and variations are salts of sulfasalazine, such as arginate and the like, gluconate, and galacturonate. Certain of the compounds of the present invention can exist in unsolvated forms as well as solvated forms, including hydrated forms, and are intended to be within the scope of the present invention. Certain of the above compounds may also exist in one or more solid or crystalline phases or polymorphs, the variable biological activities of such polymorphs or mixtures of such polymorphs are also included in the scope of this invention.

The invention also provides methods for treatment of P-MS, ALS or other neurodegenerative diseases comprising administering pharmaceutical compositions comprising effective amounts of inhibitors of system x_{c}⁻ other than sulfasalazine. In various embodiments, inhibitors of system x_{c}⁻ include, but are not limited to (S)-4-carboxyphenylglycine, 2-hydroxy-5-((4-(N-pyridin-2-ylsulfamoyl)phenyl)ethynyl)benzoic acid, aminoadipate (AAA), 4-(1-(2-(3,5-bis(trifluoromethyl)phenyl)hydrazono)ethyl)-5-(4 (trifluoromethyl)benzyl)isoxazole-3-carboxylic acid, 5-benzyl-4-(1-(2-(3,5-bis(trifluoro-methyl)phenyl)hydrazono)ethyl)isoxazole-3-carboxylic acid, and 2-hydroxy-5-[2-[4-[(3-methylpyridin-2-yl)sulfamoyl]phenyl]ethynyl] benzoic acid. Formulations of pharmaceutical compositions comprising these inhibitors can be generated by various methods, including those described in Remington, cited above.

### Administration:

In various embodiments, pharmaceutical compositions of the invention may be administered to patients by oral dosing. In certain embodiments, the pharmaceutical composition comprising sulfasalazine is formulated such that the oral bioavailability of the sulfasalazine is higher than that of crystalline sulfasalazine or than the current on-market formulation of sulfasalazine. In various embodiments, pharmaceutical compositions of the invention may be administered to a patient by various routes such as intravenously, intramuscular, buccal and rectal administration. Suitable formulations for each of these methods of administration may be found in, for example, Remington, cited above.

The specific dose of a pharmaceutical composition of the invention administered to a patient may be determined considering the various circumstances of the patient being treated such as the route of administration, the formulation of the pharmaceutical composition, the patient's medical history, the weight of the patient, the age and sex of the patient, and the severity of the condition being treated. In some embodiments, the patient is administered a pharmaceutical composition comprising sulfasalazine where in the amount of sulfasalazine is between 10 to 20,000 milligrams, 100 to 20,000 milligrams (mg)/dose; between 200 and 10,000 mg/dose; between 400 and 4000 mg/dose; or between 500 and 2,000 mg/dose.

The frequency of administration of a pharmaceutical composition of the invention to a patient may be determined considering the various circumstances of the patient being treated such as the route of administration, the formulation of the pharmaceutical composition, the patient's medical history, the weight of the patient, the age and sex of the patient, the rate of disease progression and the severity of the condition being treated. In some embodiments, the patient is administered a dose of the pharmaceutical composition more than once; once a day; or twice a day, three times a day, or four times a day. In some embodiments, a patient is administered a dose of the pharmaceutical composition of the invention less frequently than once a day, e.g., once every two days or once a week.

The length of treatment by the methods of the invention may be determined considering the various circumstances of the patient being treated such as the patient's medical history, the weight of the patient, the age and sex of the patient, the rate of disease progression and the severity of the condition being treated. In some embodiments, the patient is treated for the rest of their lifetime; or is treated for as long as the disease is active. In some embodiments, the patient is treated for less than one month; or for more than one month, e.g., for one year.

In some embodiments, pharmaceutical compositions of the invention are administered to a patient in combination with one or more other drug compositions. Such one or more other drug compositions may be administered concurrently with pharmaceutical compositions of the invention or may be administered at separate times. In certain embodiments, the one or more other drug compositions are formulated into pharmaceutical compositions of the invention. In other embodiments, the one or more drug composition and the pharmaceutical composition of the invention are administered as separate compositions. In some embodiments, Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast is administered in combination with a pharmaceutical composition of the invention to patients with P-MS. In certain of those embodiments, Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast is administered in combination with a pharmaceutical composition of the invention comprising sulfasalazine. In certain embodiments, Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast is administered to a patients with P-MS in combination with a pharmaceutical composition comprising sulfasalazine, an ABCG2 inhibitor and optionally, PVP VA64, wherein when present, the ratio of the sulfasalazine to PVP VA64 in the composition is about 20:80 wt/wt to 50:50 wt/wt. In some embodiments, riluzole is administered in combination with a pharmaceutical composition of the invention to patients with ALS. In certain embodiments, riluzole is administered in combination with a pharmaceutical composition comprising sulfasalazine. In certain embodiments, riluzole is administered to patients with ALS in combination with a pharmaceutical composition comprising sulfasalazine, an ABCG2 inhibitor and optionally, PVP VA64, wherein when present, the ratio of the sulfasalazine to PVP VA64 in the composition is about 20:80 wt/wt to 50:50 wt/wt. In some embodiments, Brivaracetam, Carbamazepine, Clobazam, Clonazepam, Diazepam, Divalproex Sodium, Epidiolex, Eslicarbazepine Acetate, Ethosuximide, Ezogabine, Felbamate, Gabapentin, Lacosamide, Lamotrigine, Levetiracetam, Lorazepam, Oxcarbazepine, Perampanel, Phenobarbital, Phenytoin, Pregabalin, Primidone, Rufinamide, Tiagabine Hydrochloride, Topiramate, Valproic Acid, Vigabatrin or Zonisamideis administered in combination with a pharmaceutical composition of the invention to patients with seizure disorders. In certain of those embodiments, Brivaracetam, Carbamazepine, Clobazam, Clonazepam, Diazepam, Divalproex Sodium, Epidiolex, Eslicarbazepine Acetate, Ethosuximide, Ezogabine, Felbamate, Gabapentin, Lacosamide, Lamotrigine, Levetiracetam, Lorazepam, Oxcarbazepine, Perampanel, Phenobarbital, Phenytoin, Pregabalin, Primidone, Rufinamide, Tiagabine Hydrochloride, Topiramate, Valproic Acid, Vigabatrin or Zonisamideis administered in combination with a pharmaceutical composition comprising sulfasalazine and an ABCG2 inhibitor. In certain embodiments, Brivaracetam, Carbamazepine, Clobazam, Clonazepam, Diazepam, Divalproex Sodium, Epidiolex, Eslicarbazepine Acetate, Ethosuximide, Ezogabine, Felbamate, Gabapentin, Lacosamide, Lamotrigine, Levetiracetam, Lorazepam, Oxcarbazepine, Perampanel, Phenobarbital, Phenytoin, Pregabalin, Primidone, Rufinamide, Tiagabine Hydrochloride, Topiramate, Valproic Acid, Vigabatrin or Zonisamide is administered to a patients with seizure disorders in combination with a pharmaceutical composition comprising sulfasalazine, an ABCG2 inhibitor and optionally, PVP VA64, wherein when present, the ratio of the sulfasalazine to PVP VA64 in the composition is about 20:80 wt/wt to 50:50 wt/wt.

### EXAMPLES

The following examples below are intended to be purely exemplary of the invention and should not be considered to limit the invention in any way. The examples are not intended to represent that the experiments below are all or the only experiments performed. Efforts have been made to ensure accuracy with respect to numbers used but some experimental errors and deviations should be accounted for. Unless otherwise indicated, parts and percentages are by weight, temperature in degrees Celsius (° C.) and pressure is at or near atmosphere. These examples may be employed for the preparation of the compositions and formulation of the present application.

### Example 1: Treatment with Sulfasalazine Increases Absolute Survival and Increases the

Lifespan of SOD1 Mice after Onset of Definitive Neurological Disease: The following experiments demonstrate that treatment with sulfasalazine: (1) increased the absolute lifespan of SOD1 mice, and (2) extended life span of SOD1 mice after onset of definitive neurological disease. This latter survival parameter is relevant to human patients, who typically will not begin therapy until after definitive diagnosis of ALS.

High-copy SOD1^{G93A} transgenic mice were derived from the B6SJL-TgN(SOD1G93A)1Gur strain, obtained from The Jackson Laboratory (Bar Harbor, Me.) and originally produced by Gurney, e.g. Gurney et al., Science 264: 1772-1775 (1994). Animal experiments with the SOD1 model were performed at ALS Therapy Development Institute (herein “ALS-TDI”; Cambridge, Mass.). All mice were genotyped to verify copy number of the SOD1 transgene. Animal handling and study protocols were as previously described by ALS-TDI, e.g. Scott et al., Amyotroph. Lateral Scler. 9: 4-15 (2008).

Groups were balanced with respect to gender and body weight within gender. In addition, groups were age-matched and littermate-matched. Each male and female in the drug treatment group had a corresponding male and female littermate in the vehicle control group. A total of 59 mice were used in the study, divided into 2 cohorts as shown in Table 1. Each cohort of was balanced between males and females.

TABLE 1
Cohorts used in the survival study

| Cohort | Genotype | Treatment | Male/Female |
|---|---|---|---|
| 1 (n = 32) | SOD1 | Vehicle Control | 16/16 |
| 2 (n = 27) | SOD1 | Sulfasalazine | 14/13 |
| | | (Drug Treatment) | |

Starting at an age of 50 days, mice were administered sulfasalazine or saline two times per day (8 hours apart), 7 days per week at a dose of 200 mg/kg. Sulfasalazine was prepared by weighing 100 mg of compound into a 50 mL corning tube. 5 mL of 0.1 N NaOH was added and the tube gently sonicated. Approximately 140 μL of 1 N HCl was then added to bring the pH to 8.00. The resulting 20 mg/mL solution was delivered by intraperitoneal injection at 10 ml/kg. Vehicle treated mice were administered saline.

An ABCG2 inhibitor, such as TPGS or Tween 20, may be formulated with sulfasalazine to prepare the formulations as described herein. In addition, the sulfasalazine formulations may be dosed orally as a formulation with or without an excipient.

Neurological scores were assessed daily from day 50 for both hind legs. The neurological score was based on a scale of 0 to 4. Criteria used to assign each score level are from Scott et al., Amyotroph. Lateral Scler. 9: 4-15 (2008) and are described in Table 2.

TABLE 2
Criteria for assigning neurological scores

| Score | Criteria |
|---|---|
| 0 | No ALS symptomology. Full extension of hind legs away from lateral midline when |
| | mouse is suspended by its tail, and mouse can hold this for 2 seconds, suspended 2-3 |
| | times. |
| 1 | Initial Pre-ALS symptomology. Collapse or partial collapse of leg extension towards |
| | lateral midline (weakness) or trembling of hind legs during tail suspension. |
| 2 | Definitive neurological disease. Toes curl under at least twice during walking 2 paper |
| | towel lengths (≈12 inches), or any part of foot is dragging along cage bottom/table. |
| 3 | Advanced disease. Rigid paralysis or minimal joint movement, foot not being used for |
| | forward motion. |
| 4 | End stage. Mouse cannot right itself within 30 seconds from either side. |

The date of definitive neurological disease was the day that the mouse first scored a “2” on the Neurological Score. Upon reaching a score of Neurological Score of “4”, mice were euthanized and the date of death was recorded.

Sulfasalazine had no statistical effect on time of disease onset. Treatment with sulfasalazine increased median absolute survival of the SOD1 mice by 3.5 days, with a p-value of p=0.07 using the Cox proportional hazard likelihood ratio (FIG. 1). While the effect of sulfasalazine on absolute survival is modest, it is 68% greater than riluzole, the only approved therapy for ALS, when tested in the same SOD1 model under similar conditions (sulfasalazine, 2.7% increased absolute lifespan vs. riluzole, 1.6% increased absolute lifespan; see e.g. Lincecum et al., Supplementary Material, Nat. Genetics 42: 392-411 (2010)).

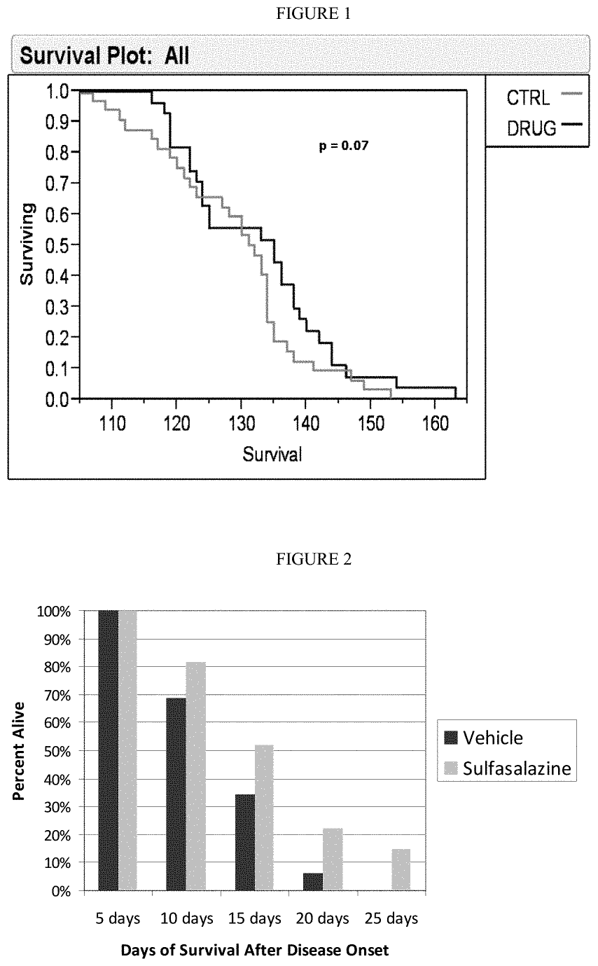

The above sulfasalazine formulation (referred to as “sulfasalazine formulation”) had a much stronger effect on survival after onset on definitive neurological disease. Survival after onset of definitive neurological disease is defined as the total number of days the mice lived after reaching definitive neurological disease (Neurological Score of 2) and before death (Neurological Score of 4). Survival after onset of definitive neurological disease was analyzed in two ways. The first analysis used the mean ages of definitive disease onset and death to calculate the mean lifespan of the SOD1 mice after disease onset, with and without sulfasalazine treatment. The mean lifespan of the SOD1 mice after onset of definitive neurological disease is shown in Table 3. A histogram plot of the survival of the SOD1 mice treated with vehicle and sulfasalazine following onset of definitive neurological disease is shown in FIG. 2. The analyses showed that sulfasalazine formulation treated mice lived, on average, 39% longer than vehicle treated mice after onset of definitive neurological disease (p=0.02, t-test with Welch's correction for unequal variances). The 95% confidence interval ranged from a lifespan increase of 21% to 52% compared to the untreated mice.

TABLE 3
Mean lifespan after onset of definitive neurological disease.

| | | | Total Days of | | |
|---|---|---|---|---|---|
| | Mean Day of | | Survival | | |
| | Definitive | | after Onset of | Lower 95% | Upper 95% |
| | Neurological | Mean Day of | Definitive | Confidence | Confidence |
| Group | Disease | Death | Disease | Interval | Interval |
| Vehicle | 116.4 | 128.9 | 12.5 | 10.73 | 14.27 |
| Sulfasalazine | 115.2 | 132.6 | 17.4 | 12.94 | 21.73 |
| Absolute Change | −1.2 | 3.7 | 4.9 | 2.21 | 7.46 |
| Percent Change | −1.0% | 2.9% | 39.2% | 20.6% | 52.3% |
| p-value | p = 0.64 | p = 0.12 | p = 0.02 | | |

The second method used to analyze the survival data was to compare the expected and observed number of days the sulfasalazine formulation treated mice spent in one of 3 disease categories to determine if there was a significant difference between the expected and observed values. The first day that a neurological score was determined was day 50 of age; measurements were collected daily afterwards until death.

The three categories were: (1) days spent before definitive neurological disease, e.g. with a neurological score of 0 or 1; (2) days spent during definitive neurological disease, e.g. with a neurological score of 2 or 3; and (3) days at death, e.g. with a neurological score of 4. By definition, mice were only in the dead state for 1 day, as they were euthanized upon reaching a neurological score of 4; this category was included as a positive control to ensure the integrity of the data and analysis.

The expected distribution of days the sulfasalazine formulation treated mice spent in each of these three disease categories was calculated from the observed distribution of the vehicle treated mice, with the null hypothesis that sulfasalazine treatment had no effect on the distribution. The observed distribution of sulfasalazine treated mice is based on daily scoring and is normalized to the number of mice in the groups.

The results of this analysis are shown in Table 4. The sulfasalazine formulation treated mice, as a group, survived a total of 112 days longer than expected after onset of definitive neurological disease (neurological score of 2 or 3). This result was highly significant by Chi-Square analysis, with a p-value of less than 0.0004 by the Wald test and 0.0003 by the Likelihood ratio test. There was no significant difference in the total days spent in the pre-disease state (neurological score of 0 or 1) or death (neurological score of 4).

TABLE 4
Expected and observed distribution of sulfasalazine formulation neurological scores
and calculated p-values.

| | | | | | | | Effect |
|---|---|---|---|---|---|---|---|
| | | | | Observed | Observed | Effect | (Likelihood |
| | | | Expected Days, | Days, | Days Minus | (Wald Tests)² | Ratio Tests)² |
| Measurement | Disease Category | Vehicle | Sulfasalazine¹ | Sulfasalazine | Expected Days | Prob > ChiSq | Prob > ChiSq |
| Total Days | Before definitive disease onset | 2041 | 1722 | 1708 | −14 | 0.0004 | 0.0003 |
| | (neurological score = 0 or 1) | | | | | | |
| | Definitive neurological disease | 357 | 301 | 413 | 112 | | |
| | (neurological score = 2 or 3) | | | | | | |
| | Death (neurological score = 4) | 32 | 27 | 27 | 0 | | |
| | Total Observations (Days) | 2430 | 2050 | 2148 | 98 | | |

¹Expected days for sulfasalazine treated animals if sulfasalazine has no effect on disease; values predicted from vehicle-treated cohort and normalized for number of animals in treated cohort.
²Prob > ChiSq is the probability of obtaining a greater Chi-square value by chance alone if treatment has no effect on time spent within each disease category.
All statisics performed using the JMP10 statistics program (SAS Institute).

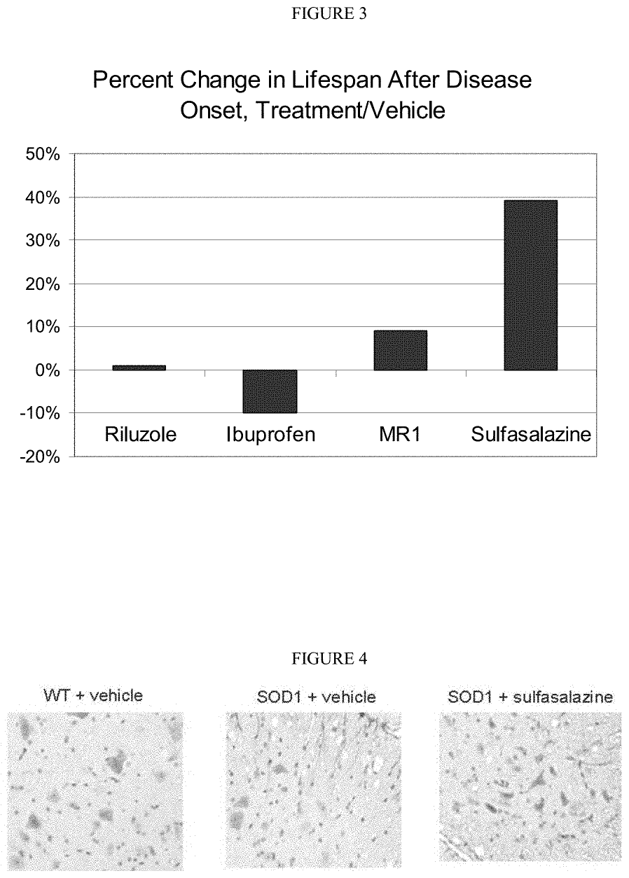

The increased lifespan following onset of definitive neurological disease seen with sulfasalazine formulation was also compared to published results of other compounds tested in the SOD1 mouse model. FIG. 3 shows the percent difference in survival following onset of neurological disease for sulfasalazine formulation, two general anti-inflammatory compounds (ibuprofen and MR1, an antibody to CD40L) and riluzole, the only drug currently approved for ALS. Sulfasalazine formulation increased lifespan by 39%, anti-CD40L increased lifespan by 9%, riluzole increased lifespan by 1% and ibuprofen decreased lifespan after onset of neurological disease by 10%. See, e.g. Shin et al., J. Neurochem. 122: 952-961 (2012); Lincecum et al., Nat. Genetics 42: 392-411 (2010).

This comparison illustrates that the increased lifespan observed with sulfasalazine formulation is significantly larger than is observed with other tested compounds, including two general anti-inflammatory compounds (ibuprofen and anti-CD40L) and the only approved therapy for ALS (riluzole).

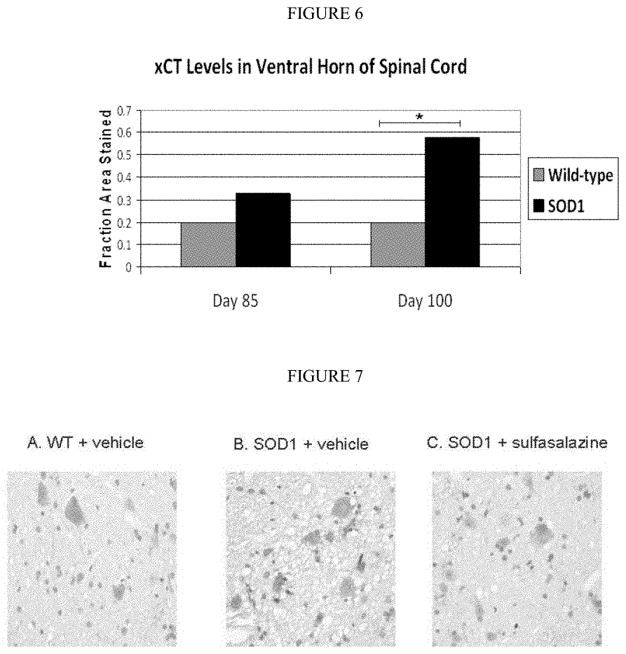

The experiments in the SOD1 animal model of ALS demonstrate that, while the effect of sulfasalazine formulation on absolute survival is modest, it was superior to riluzole, the only approved therapy for ALS. Importantly, the benefit in survival by sulfasalazine formulation after onset of definitive neurological disease is large, in terms of absolute size (approximately 40%), the statistical significance and when compared to other compounds, including riluzole. It is noteworthy that the entire increase in survival noticed in the absolute survival analysis (median 3.7 days) occurs after the definitive onset of neurological disease. This result is consistent with the expression data (FIG. 6, below) that shows xCT expression escalates with disease progression. Based on the expression profile of the target, it is expected that sulfasalazine formulation would have little effect on delaying the onset of disease, but would have a progressively beneficial effect as disease progresses, as was observed in the survival study.

These experiments demonstrate that sulfasalazine formulation has modest efficacy on absolute survival and strong efficacy on survival after onset of definitive neurological disease in the SOD1 mouse model of ALS. As most ALS patients do not begin therapy until after diagnosis of neurological disease, the latter measurement is especially relevant to the treatment of human disease.

### Example 2: Expression of xCT (SLC7A11) is Elevated in the Spinal Cord of SOD1 Mice

The following studies used quantitative immunohistochemistry to determine: (1) if the expression of xCT in the spinal cord was elevated in SOD1 mice, (2) if so, whether xCT over-expression increased with disease progression, and (3) whether treatment with sulfasalazine affected xCT expression in the spinal cord of SOD1 mice.

Two ages of mice were chosen for this analysis: day 85, when SOD1 mice show no overt sign of the ALS-like symptomology, and day 100, when SOD1 mice typically begin displaying the first signs of ALS-like symptomology, such as partial collapse of leg extension towards lateral midline (weakness) or trembling of hind legs during a tail suspension test. For the immunohistochemical studies, a total of 48 mice were divided into 6 cohorts of 8 mice each (4 females and 4 males) as shown in Table 5.

TABLE 5
Cohorts used in the Immunohistochemical Studies

| Cohort | | | Age of mouse |
|---|---|---|---|
| (n = 8) | Genotype | Treatment | at sacrifice |
| 1 | Wild-type | vehicle | 85 days |
| 2 | SOD1 | vehicle | 85 days |
| 3 | SOD1 | sulfasalazine | 85 days |
| 4 | Wild-type | vehicle | 100 days |
| 5 | SOD1 | vehicle | 100 days |
| 6 | SOD1 | sulfasalazine | 100 days |

Starting at an age of 50 days, mice were administered sulfasalazine formulation or saline two times per day (8 hours apart), 7 days per week at a dose of 200 mg/kg. Sulfasalazine formulation was prepared by weighing 100 mg of compound into a 50 mL coming tube. 5 mL of 0.1 N NaOH was added and the tube gently sonicated. Approximately 140 μL of 1 N HCl was then added to bring the pH to 8.00. The resulting 20 mg/mL solution was delivered by intraperitoneal injection at 10 ml/kg. Vehicle treated mice were administered saline.

Mice were sacrificed by CO₂ asphyxiation according to IACUC approved protocols. The spinal cord was extruded with cold PBS into a bath of cold PBS from the vertebral column of mice using an 18 gauge needle inserted in the sacral vertebral column to a friction fit. Upon extrusion, the spinal cord tissue was rinsed and dropped into 4% paraformaldehyde for 24 hours at room temperature (RT, approximately 25° C.). The tissue was then transferred to a 1× phosphate buffered saline (PBS) solution. Samples were then processed by TissueTek processors for paraffin embedding. Spinal cord samples were then embedded in paraffin blocks and oriented for transverse sectioning. Spinal cord samples were sectioned at 10 microns thickness. Three representative sections were cut from lumbar, thoracic and cervical regions of the spinal cord. Samples were pretreated with Pronase for 20 minutes at RT, followed by treatment with 3% H₂O₂ for 12 minutes at RT. Horse serum was added to 2% and samples incubated for 20 minutes at RT. The samples were then incubated with the primary antibody (Anti-xCT; purchased from Abcam (Cambridge, Mass.); Catalog #Ab37185; diluted 1:500 in PBS) overnight at 4° C. The secondary antibody (Biotin labeled goat anti-rabbit IgG; 1:500 dilution in PBS) was then added and the reaction incubated overnight at RT. Reaction products were developed using the Vector ABC system (Vector Labs, Burlingame, Calif.) using avidin-conjugated horseradish peroxidase. xCT expression was visualized by addition of the chromogenic substrate DAB (3,3′-diaminobenzidine) for 10 minutes at RT. Each stained section was imaged at objectives: 4×, 10×, 20× and 40×. For each objective image, light parameters were optimized and kept consistent across all sections. For SLC7A11 analysis, all images that were captured at 20× were then imported into ImageJ freeware (NIH, Bethesda, Md.). A maximum entropy threshold algorithm was applied to all images in a completely blinded fashion to filter out all pixels that were not stained as DAB positive. The key parameter measured and reported is area fraction. Area fraction is the proportion of total pixels that are DAB positive in the ventral horn of the spinal cord. All statistical analyses were performed using JMP® 7.0, SAS Institute, Inc. Area fraction was compared with respect to treatment using 1-way ANOVA analysis, with a p-value of 0.05 considered significant.

FIG. 4 shows representative images from day 100 mice. Increased expression of xCT is visible in the sections from the SOD1 mice, with and without sulfasalazine formulation treatment.

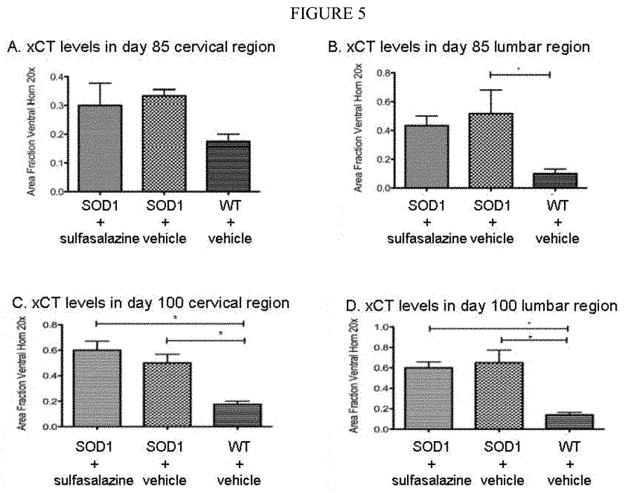

FIG. 5 shows the quantitation of xCT area fraction for the cervical and lumbar regions of the spinal cord in day 85 and day 100 mice. At day 85, xCT protein levels were elevated in both the cervical and lumbar regions in SOD1 mice, reaching statistical significance (p<0.05) in the lumbar region (FIG. 5, panel B). At day 100, xCT protein levels were elevated in both the cervical and lumbar regions in SOD1 mice, reaching statistical significance (p<0.05) in both regions (FIG. 5, Panels C and D).

FIG. 6 compares total xCT expression levels in the ventral horn of the spinal cord in day 85 and day 100 mice. For this analysis, values for the cervical, thoracic and lumbar regions were combined into a single value. On day 85 SOD1 mice, xCT protein levels were elevated by approximately 50% in the combined spinal cord sections compared to day 85 wild-type mice. On day 100 SOD1 mice, xCT protein levels were elevated by approximately 300% across in the combined spinal cords sections compared to day 100 wild-type mice.

These results demonstrate that (1) xCT target expression is elevated in the diseased tissue—the ventral portion of the spinal cord—of SOD1 mice; (2) expression of xCT escalates significantly during disease progression in the SOD1 mice (day 85 versus day 100, FIG. 6), and (3) treatment with sulfasalazine formulation did not have a significant effect on xCT expression (FIG. 5, Panels A-D). There does not appear to be a compensatory or rebound effect on xCT levels when it is inhibited by sulfasalazine. Such a rebound effect could lead to loss of efficacy upon treatment.

The increased expression of xCT observed during disease progression is consistent with sulfasalazine formulation having greatest efficacy during the later stages of disease.

### Example 3: Sulfasalazine Formulation Reduces Levels of Neuroinflammatory Cells in the Spinal Cord of SOD1 Mice

The following experiments employed quantitative immunohistochemistry to: (1) compare the neuroinflammatory cell populations in the spinal cord of SOD1 mice to the cell populations in wild-type mice, and (2) test whether the treatment with sulfasalazine formulation decreases neuroinflammatory cell populations in the spinal cord of SOD1 mice.

The same test mice, spinal cord preparations and methods of analysis used in the neuroinflammatory study were identical to those used in the xCT quantitation study. Two neuroinflammatory cell populations were quantitated: (1) activated microglial cells using an antibody to the F4/80 antigen, and (2) activated astrocytes, using an antibody to the GFAP antigen. For each objective image, light parameters were optimized and kept consistent across all sections. Images that were captured at 20× were then imported into ImageJ freeware (NIH, Bethesda, Md.). A maximum entropy threshold algorithm was applied to all images in a completely blinded fashion to filter out all pixels that were not stained as DAB positive. 20× images were analyzed in a blinded fashion and mean area fraction occupied by stain was tabulated. Levels of neuroinflammation in the spinal cord were assessed by measuring the area fraction of the area of the ventral horn in the spinal cord occupied by activated astrocytes or microglia. Area fraction was compared with respect to treatment using 1-way ANOVA analysis, with a p-value of 0.05 considered significant.

For quantitation of microglial activation, samples were pretreated with Pronase for 20 minutes at RT, followed by treatment with 3% H₂O₂ for 12 minutes at RT (25° C.). Goat serum was added to 2% and samples incubated for 20 minutes at RT. The samples were then incubated with the primary antibody (Anti-F4/80) purchased from Serotec (Catalog # MCA497R; Oxford, United Kingdom), diluted 1:250 in PBS overnight at 4° C. The secondary antibody (Biotin labeled goat anti-rabbit IgG; 1:250 dilution in PBS) was then added and the reaction incubated 1 hour at RT. Reaction products were developed using the Vector ABC system (Vector Labs, Burlingame, Calif.) using avidin-conjugated horseradish peroxidase (45 minutes at RT). Activated microglia were visualized by addition of the chromogenic substrate DAB (3,3′-diaminobenzidine) for 6 minutes at RT.

For quantitation of astrocyte activation, samples were pretreated with heated citrate buffer for 20 minutes, followed by treatment with 3% H₂O₂ for 12 minutes at RT. Horse serum was added to 2% and samples incubated for 20 minutes at RT. The samples were then incubated with the primary antibody (Anti-GFAP) purchased from Abcam (Catalog # Ab10062; Cambridge, Mass.), diluted 1:1000 in PBS overnight at 4° C. Reaction products were developed using the Vector ImmPress system (Vector Labs, Burlingame, Calif.) using an anti-mouse IgG-conjugated horseradish peroxidase. Activated astrocytes were visualized by addition of the chromogenic substrate DAB (3,3′-diaminobenzidine) for 90 seconds at RT.

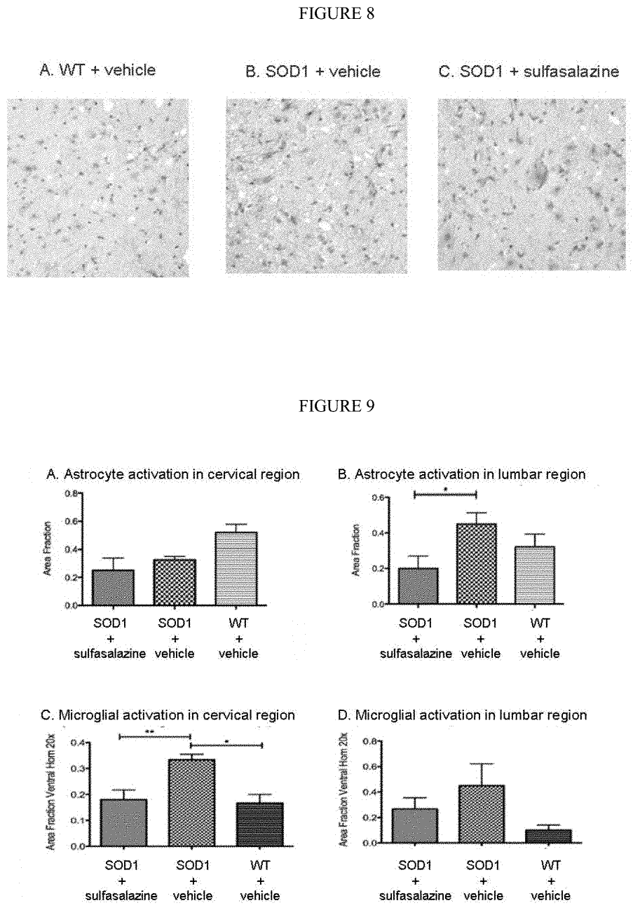

FIG. 7 shows representative images from tissue from day 85 mice stained for activated microglia. FIG. 8 shows representative images from tissue from day 100 mice stained for activated astrocytes.

FIG. 9 shows area fraction quantitation of the activated astrocytes and microglial cells in the ventral horn from the cervical and lumbar regions of the spinal cord in day 85 mice. A strong trend toward astrocyte activation was observed in diseased mice (SOD1) compared to non-diseased mice (WT) in the lumbar region (FIG. 9, Panel B). Sulfasalazine formulation treatment significantly lowered astrocyte activation in the lumbar region (FIG. 9, Panel B). Astrocyte activation was not elevated in the cervical region in SOD1 mice vs. WT mice (FIG. 9, Panel A).

In day 85 mice, increased microglial activation was observed in diseased mice (SOD1) compared to non-diseased mice (WT) in both the cervical region (FIG. 9, Panel C) and in the lumbar region (FIG. 9, Panel D), although activation in the lumbar region did not reach statistical significance. Sulfasalazine formulation treatment significantly decreased microglial activation in the cervical region of SOD1 mice (FIG. 9, Panel C) and also reduced microglial activation in the lumbar region in SOD1 mice, although this effect did not reach statistical significance.

These results demonstrate that in day 85 SOD1 mice: (1) increased levels of neuroinflammatory cells (activated astrocytes and microglia) are present in the spinal cord before ALS-like symptomology is observed, and (2) treatment with sulfasalazine formulation lowers the overall levels of neuroinflammatory cells (activated astrocytes and/or microglia) in both the cervical and lumbar regions of the spinal cord.

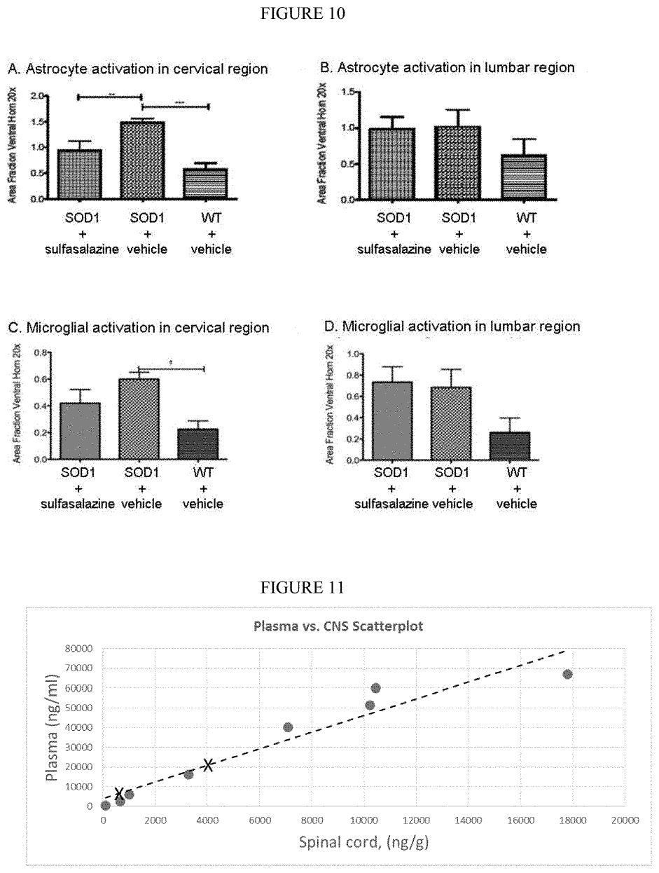

FIG. 10 shows area fraction quantitation of the activated astrocytes and microglial cells in the ventral horn from the cervical and lumbar regions of the spinal cord in day 100 mice. Significantly increased astrocyte activation was observed in diseased mice (SOD1) compared to non-diseased mice (WT) in the cervical region (FIG. 10, Panel A). Sulfasalazine formulation treatment significantly lowered astrocyte activation in the cervical region (FIG. 10, Panel A). In the lumbar region, there was a trend towards increased astrocyte activation in the lumber region, but it was not statistically significant. Sulfasalazine formulation treatment did not affect astrocyte activation in the lumbar region (Panel B).

At day 100, increased microglial activation was observed in diseased mice (SOD1) compared to non-diseased mice (WT) in the cervical region (FIG. 10, Panel C) and in the lumbar region (FIG. 10, Panel D), although activation in the lumbar region did not reach statistical significance. Sulfasalazine formulation treatment resulted in a trend towards decreased microglial activation in the cervical region (FIG. 10, Panel C) and did not affect such activation in the lumbar region (FIG. 10, Panel D).

Results demonstrate that in Day 100 SOD1 mice: (1) increased levels of neuroinflammatory cells (activated astrocytes and microglia) are present in the spinal cord, in particular the cervical region, and (2) treatment with sulfasalazine formulation lowers the overall levels of neuroinflammatory cells in the cervical region of the spinal cord.

Table 6 contains a summary of all the data from the neuroinflammation experiment presented in tabular format. The changes in area fraction staining in the cervical, thoracic, and lumbar regions of the spinal cord, as well as the combined changes across the whole spinal cord (sum of cervical, thoracic and lumbar regions) are scored for the following group comparisons: (1) Whether increased activation of microglia and astrocytes was observed in diseased (SOD1, vehicle treated) mice vs. non-diseased (wild-type) mice (Column 4). In a total of 16 measurements, evidence for astrocyte and/or microglial activation was observed 14 times, reaching statistical significance 5 times; and (2) whether treatment with sulfasalazine formulation decreased activation of microglia and astrocytes compared to vehicle treatment in SOD1 mice (Column 5). From the total of 14 tissues that showed activation of astrocytes and/or microglia in SOD1 mice, sulfasalazine formulation treatment was observed to decrease activation 8 times, reaching statistical significance 4 times.

TABLE 6
Summary of Neuroinflammation Data

| | | | Column 4: Increased | Column 5: Decreased activation in |
|---|---|---|---|---|
| | | | activation in SOD1 | sulfasalazine formulation treated |
| Tissue | Day | Cell type | (vehicle) vs. WT mice | SOD1 vs. vehicle treated SOD1 mice |
| Cervical | 85 | Astrocytes | No increase | No decrease |
| Cervical | 100 | Astrocytes | Strong increase (p < 0.001) | Strong decrease (p < 0.01) |
| Thoracic | 85 | Astrocytes | Trend | Trend |
| Thoracic | 100 | Astrocytes | Trend | No decrease |
| Lumbar | 85 | Astrocytes | Trend | Decrease (p < 0.05) |
| Lumbar | 100 | Astrocytes | Trend | No decrease |
| Combined | 85 | Astrocytes | Trend | Decrease (p < 0.05) |
| Combined | 100 | Astrocytes | Trend | Trend |
| Cervical | 85 | Microglia | Increase (p < 0.05) | Strong decrease (p < 0.01) |
| Cervical | 100 | Microglia | Increase (p < 0.05) | Trend |
| Thoracic | 85 | Microglia | No increase | No decrease |
| Thoracic | 100 | Microglia | Trend | No decrease |
| Lumbar | 85 | Microglia | Trend | Trend |
| Lumbar | 100 | Microglia | Trend | No decrease |
| Combined | 85 | Microglia | Increase (p < 0.05) | Trend |
| Combined | 100 | Microglia | Increase (p < 0.05) | No decrease |

This experiment establishes that sulfasalazine formulation treatment lowers the levels of both activated microglial cells and activated astrocytes in the spinal cord. The results from the neuroinflammatory study suggest that xCT activity is required for maximum levels of neuroinflammation to occur.

### Example 4: Sulfasalazine Reaches Therapeutic Concentrations in the Spinal Cord and Spinal Cord Levels are Proportional to Concentrations in the Plasma

The experimental procedures and results provided below demonstrate the exposure and pharmacokinetics of sulfasalazine in the spinal cord and plasma of SOD1 mice.

### Study Protocol and Sample Analysis:

SOD1 mice were dosed with sulfasalazine formulation at 200 mg/kg intraperitoneally and spinal cords and plasma (50 μl) harvested at indicated times (n=3 mice per time point). The zero time point was taken before drug was administered to serve as a negative control for drug quantitation. Analytical methods were developed and performed by MicroConstants (San Diego, Calif.). Spinal cords and blood plasma samples (50 μl) were homogenized in 150 μl of phosphate buffer and then extracted by a mixture of methylene chloride and MTBE (1:4 dilution). Sample extracts were analyzed and quantitated by high-performance liquid chromatography using a BetaMax Acid column maintained at 35° C. The mobile phase was nebulized using heated nitrogen in a Z-spray source/interface and the ionized compositions were detected and identified using a tandem quadrupole mass spectrometer (MS/MS).

### Analytical Method Qualification:

A reference standard of sulfasalazine (Sigma-Aldrich, Catalog # S0883) was used to generate a standard curve in rat plasma. The assay gave a linear response to concentrations of sulfasalazine from 10 to 20,000 ng/ml (Table 7). Dilution controls showed that samples could be diluted up to 1:100 and give a linear response in the assay. Curve fitting from this data generated the following parameters for the equation used to calculate unknown concentrations:

LOG(y)=A+B*LOG(x) General Equation:

where y=peak height ratio and x=concentration

Specific Parameters for sulfasalazine: A=3.06, B=0.976; Correlation coefficient=1.00

TABLE 7
Standard Curve Values of Sulfasalazine.

| | Standard Concentrations (ng/mL) | | | | | | | | | | | | |
|---|---|---|---|---|---|---|---|---|---|---|---|---|---|
| Analyte | 10.0 | | 20.0 | 50.0 | 100 | 250 | 500 | 1,000 | 2,000 | 5,000 | 10,000 | 20,000 | |
| sulfasalazine | 9.80 | 9.88 | 18.5 | 46.4 | 109 | 255 | 519 | 1,100 | 2,150 | 5,360 | 10,200 | 17,000 | 18,100 |
| (Measured) | | | | | | | | | | | | | |

| | | | | | | | | | | | |
|---|---|---|---|---|---|---|---|---|---|---|---|
| Mean (ng/mL) | 9.84 | 18.5 | 46.4 | 109 | 255 | 519 | 1,100 | 2,150 | 5,360 | 10,200 | 18,000 |
| Percent Standard | −1.60 | −7.50 | −7.20 | 9.00 | 2.00 | 3.80 | 10.0 | 7.50 | 7.20 | 2.00 | −10.0 |
| deviation | | | | | | | | | | | |

Separately, an internal standard (deuterated sulfasalazine) was used to determine compound extraction efficiency from mouse CNS (brain) tissue and plasma. The extraction efficiency of sulfasalazine was determined to be >98% from mouse brain tissue and plasma.

Table 8 shows the mean values for the concentrations of sulfasalazine in the spinal cord and in the plasma and the ratio of sulfasalazine in the spinal cord to the plasma.

TABLE 8
Mean concentrations of sulfasalazine in the CNS (spinal cord)
and plasma and ratios in CNS (spinal cord) to plasma.
BQL= below quantitative limit of detection (10 ng/ml):

| Time | Spinal cord, | Plasma, | Ratio |
|---|---|---|---|
| (min) | mean (ng/g) | mean (ng/ml) | (Spinalcord/plasma) |
| 0 | BQL | BQL | BQL |
| 5 | 17,779 | 67,143 | 27% |
| 15 | 10,419 | 60,235 | 17% |
| 30 | 10,213 | 51687 | 20% |
| 45 | 7,065 | 40,353 | 18% |
| 60 | 3,276 | 16,234 | 20% |
| 120 | 991 | 6090 | 16% |
| 180 | 653 | 2,580 | 25% |
| 240 | 88 | 630 | 14% |

Table 8 shows that sulfasalazine formulation showed immediate penetration into the spinal cord, reaching levels of approximately 18 μg/gram of tissue within 5 minutes of drug administration. The levels in the spinal cord ranged from approximately 14-27% of the levels in the plasma over the next four hours. The half-life of sulfasalazine in the spinal cord and plasma was approximately one hour, with the levels in the spinal cord proportional to the levels in the plasma. The observed half-life in the spinal cord and plasma is consistent with the reported half-life of sulfasalazine in mouse plasma, see, e.g., Zaher et al., Mol. Pharmaceutics 3: 55-61 (2005). FIG. 11 shows the concentrations of sulfasalazine in the spinal cord versus plasma in a scatterplot format. The trendline is shown as a dashed line. The minimum therapeutic level of sulfasalazine, estimated to be approximately 2 micromolar (equivalent to 800 ng/ml; assuming a conversion of 1 gram tissue=1 ml volume) and is marked with the X on the left. The X on the right marks levels of 10 μM sulfasalazine (equivalent to 4,000 ng/ml; assuming a conversion of 1 gram tissue=1 ml volume), these levels are predicted to result in significant inhibition of xCT. Corresponding concentrations of sulfasalazine in the plasma during over this range are approximately 4,000-20,000 ng/ml.

Results of the SOD1 experiments provide strong support that sulfasalazine formulation has therapeutic applications for ALS, despite the short half-life of sulfasalazine and resulting sub-optimal drug coverage in these particular studies. Levels of the target—xCT—were elevated in diseased tissue and escalated with disease progression. Treatment with sulfasalazine formulation demonstrated significant efficacy in two important components of disease: (1) survival after onset of neurological disease and, (2) attenuation of neuroinflammation.

### Example 5:Determination of Solubility of Crystalline Compound at Different pH

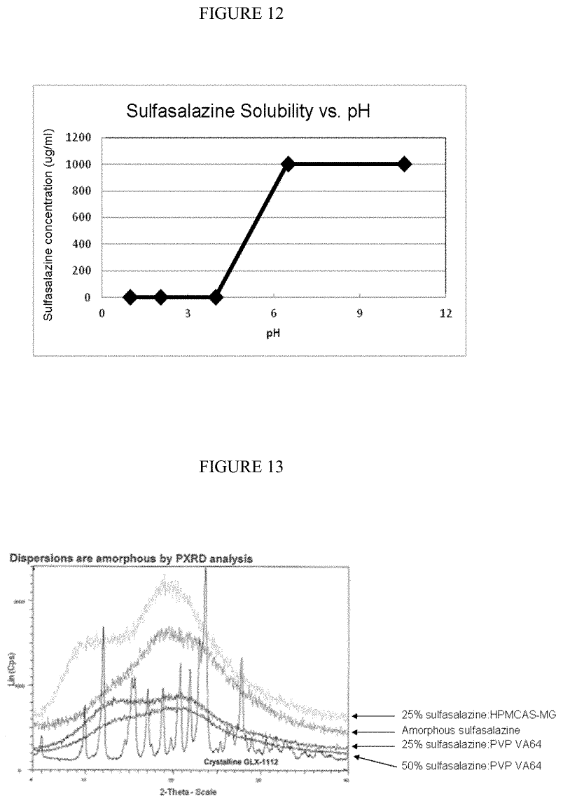

The following procedure was used to determine the effect of pH on the solubility of sulfasalazine in aqueous solutions. A 1.8 mg sample of sulfasalazine was placed in a microcentrifuge tube. A 0.9 mL of 0.01N HCl was then added to the tube, which was capped and mixed using a vortex mixer for 1 minute. The sample in the tube was then centrifuged at 15,800 relative centrifugal force (RCF) for 1 minute. A 50 μL sample of the liquid was diluted into 250 μL HPLC solvent, and the tube was capped and vortexed for 20 seconds and allowed to stand undisturbed at 37° C. until the next sample was collected. After 30 minutes, a 0.9 mL portion of buffer solution (at twice the concentration of buffer salts) was added to the microcentrifuge tube, and the procedure repeated as described above. Samples were collected at predetermined time intervals and analyzed by HPLC. The solubility of sulfasalazine as a function of pH was then determined, as shown in FIG. 12. Data indicates that crystalline drug alone may have good bioavailability based on solubility if the pH of the absorption window is high (pH 6 or above). Data also indicates that crystalline drug alone may have poor bioavailability based on solubility if the pH of the absorption window is low (below pH 6).

### Example 6

### Reformulation of Sulfasalazine Formulation to Increase Oral Bioavailability:

Novel formulations of sulfasalazine that increase the solubility of sulfasalazine at enteric pH (i.e., below pH 6) were prepared, including a formulation of sulfasalazine that increases the oral bioavailability of the sulfasalazine by at least three-fold in a rat model.

### Preparation of Sulfasalazine Formulations

### Sulfasalazine Formulation Exemplar 1: 25% Sulfasalazine: 75% HPMCAS SDD

A spray dried dispersion (SDD) of 25 wt % sulfasalazine and 75 wt % HPMCAS (hereafter “25% sulfasalazine:HPMCAS”) was prepared using a spray drying process as follows. A spray solution was prepared by dissolving 100 mg sulfasalazine and 300 mg HPMCAS (Hydroxypropylmethylcellulose acetate succinate; AQOAT M grade, Shin Etsu, Tokyo, Japan) in 19.6 gm of solvent (95/5 w/w tetrahydrofuran/water), to form a spray solution containing 2 wt % solids. This solution was spray dried using a small-scale spray-dryer, which consisted of an atomizer in the top cap of a vertically oriented 11-cm diameter stainless steel pipe. The atomizer was a two-fluid nozzle, where the atomizing gas was nitrogen delivered to the nozzle at 70° C. at a flow rate of 31 standard L/min (SLPM), and the solution to be spray dried was delivered to the nozzle at RT at a flow rate of 1.3 mL/min using a syringe pump. The outlet temperature of the drying gas and evaporated solvent was 31.5° C. Filter paper with a supporting screen was clamped to the bottom end of the pipe to collect the solid spray-dried material and allow the nitrogen and evaporated solvent to escape. The resulting spray dried powder was dried under vacuum overnight, with a yield of 89%.

### Sulfasalazine Formulation Exemplar 2: 25% Sulfasalazine:75% PVP VA64 SDD

A spray dried dispersion (SDD) of 25 wt % sulfasalazine and 75 wt % PVP VA64 (hereafter “25% sulfasalazine:PVP VA64”) was prepared using a spray drying process as follows. The procedure of sulfasalazine formulation Exemplar 1 was repeated except that the polymer was vinylpyrrolidone-vinyl acetate copolymer (PVP VA64, from BASF as Kollidon® VA 64, Ludwigshafen, Germany). The spray drying conditions were the same as sulfasalazine formulation Exemplar 1. The resulting spray dried powder was dried under vacuum overnight, with a yield of 95.7%.

A formulation comprising 25% Sulfasalazine-TPGS:75% PVP VA64 SDD is prepared according to the above procedure to provide an acceptable dried powder.

### Sulfasalazine Formulation Exemplar 3: 50% Sulfasalazine:50% PVP VA64 SDD

A spray dried dispersion (SDD) of 50 wt % sulfasalazine and 50 wt % PVP VA64 (hereafter “50% sulfasalazine:PVP VA64”) was prepared using a spray drying process as follows. A spray solution was prepared by dissolving 200 mg sulfasalazine and 200 mg PVP VA64 in 19.6 gm of solvent (90/10 w/w tetrahydrofuran/water), to form a spray solution containing 2 wt % solids. This solution was spray dried using a small-scale spray-dryer, as described in sulfasalazine formulation Exemplar 1. The resulting spray dried powder was dried under vacuum overnight, with a yield of 95.7%.

### Example 7: Characterization of the Compositions Showing Amorphous Dispersion Using PXRD Analysis

The three exemplar formulations were analyzed by powder X-ray diffraction (PXRD) using an AXS D8 Advance PXRD measuring device (Bruker, Inc. of Madison, Wis.) using the following procedure. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The X-ray source (KCu_{α}, λ=1.54 Å) was operated at a voltage of 45 kV and a current of 40 mA. Data for each sample were collected over a period of 30 minutes in continuous detector scan mode at a scan speed of 2 seconds/step and a step size of 0.04°/step. Diffractograms were collected over the 20 range of 4° to 40°. FIG. 13 shows the diffraction pattern of the formulations, revealing an amorphous halo, indicating the sulfasalazine in each of the exemplar formulations was essentially amorphous.

### Example 8: Characterization of Compositions Showing Homogeneity Using mDSC Analysis

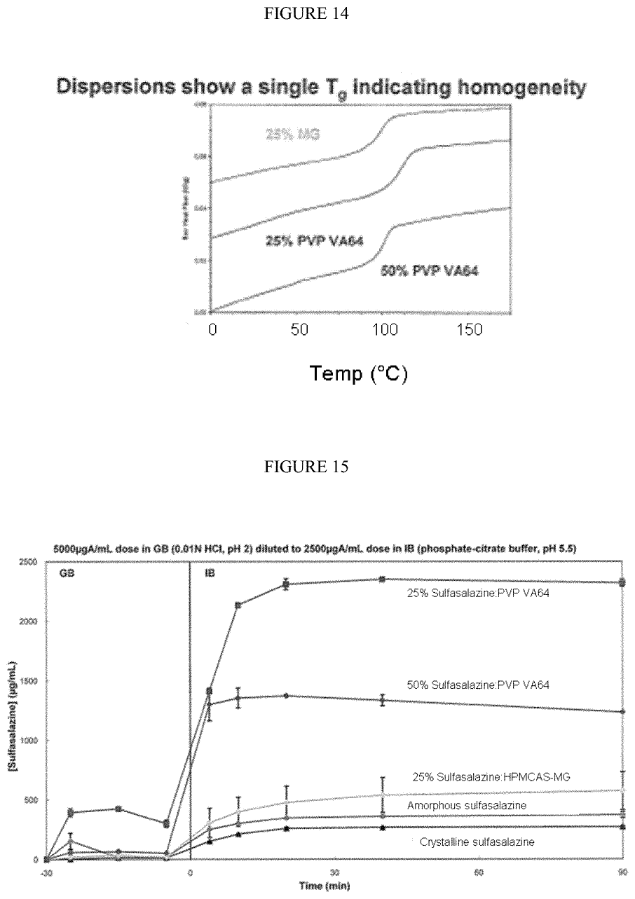

The above exemplar formulations were analyzed using modulated differential scanning calorimetry (mDSC) as follows. Samples of the formulations (about 2 to 4 mg) were equilibrated at <5% RH overnight in an environmental chamber at ambient temperature. The samples were then loaded into pans and sealed inside the environmental chamber. The samples were then analyzed on a Q1000 mDSC (TA Instruments, New Castle, Del.). Samples were scanned over the temperature range of −40° C. to 200° C., at a scan rate of 2.5° C./min, and a modulation rate of ±1.5° C./min. The glass-transition temperature (Tg) was calculated based on half height. The mDSC results are shown in FIG. 14, and the Tg is also reported in Table 9 (data are reported as an average of 3 replicates). In all cases, the dispersions exhibited a single Tg, indicating the active agent in the dispersion was molecularly dispersed and homogeneous in the SDD.

TABLE 9
Glass transition temperatures of sulfasalazine preparations

| Sample | Tg at <5% RH (° C.) |
|---|---|
| Formulation Exemplar 1 | 98.1 ± 0.3 |
| 25% Sulfasalazine:HPMCAS SDD | |
| Formulation Exemplar 2 | 110 |
| 25% Sulfasalazine-TPGS:PVP | |
| VA64 SDD | |
| Formulation Exemplar 3 | 118.0 ± 0.2 |
| 50% Sulfasalazine:PVP VA64 SDD | |

### Example 9: Determination of Solubility of Reformulated Compounds at Enteric pH

The release of sulfasalazine from the dispersions of formulation exemplars 1-3, crystalline sulfasalazine, and amorphous sulfasalazine formulation (made by spray drying) was determined using the following procedures. A sample mass of 4.5 mg of the test material was placed in a microcentrifuge tube. To this was added 0.9 mL of gastric buffer (GB) solution (0.01 N HCl, pH 2). The tubes were vortexed for one minute, then centrifuged for one minute before taking each sample. Samples (the liquid phase) were taken at 5, 15, and 25 minutes. At 30 minutes after the start of the test, 0.9 mL of intestinal buffer (IB) solution (a phosphate/citrate buffer at pH 5.5) was added to the tubes (at a double concentration of the buffer salts to result in the desired pH level and buffer strength). The tubes were vortexed for one minute, then centrifuged for one minute before taking each sample. Samples were taken at 4, 10, 20, 40, 90 and 1200 minutes after addition of the intestinal buffer solution. The concentration of sulfasalazine was determined by HPLC as previously described. Table 10 shows the data from the solubility experiment and FIG. 15 shows the data in graphical format. This data demonstrated that the amorphous sulfasalazine preparation has higher solubility than the crystalline sulfasalazine, by about 36%. When the amorphous sulfasalazine was prepared with polymers, the solubility further increased. The 25% sulfasalazine:HPMCAS-MG formulation had an increase in solubility of about 200% compared to the crystalline sulfasalazine and of about 46% over the amorphous sulfasalazine. The 50% sulfasalazine-TPGS:PVP VA64 polymer has an increase in solubility of about 500% compared to the crystalline sulfasalazine and of about 370% over the amorphous sulfasalazine. The 25% sulfasalazine-TPGS:PVP VA64 polymer has an increase in solubility of about 800% compared to the crystalline sulfasalazine and of about 640% over the amorphous sulfasalazine.

TABLE 10
Solubility of Compounds in Gastric Buffer and Intestinal Buffer

| | | | | | | Ratio of | Ratio of |
|---|---|---|---|---|---|---|---|
| | | | Cmax IB | Cmax IB | | AUC | AUC |
| | Cmax GB | Cmax IB | (ug/mL) | (ug/mL) | AUC | to cysiallIne | to amorphous |
| Sample | (ug/mL) | (ug/mL) | at 90 min | at 1200 min | (min * ug/mL) | sulfasalazine | sulfasalazine |
| Crystalline sulfasalazine | 15 | 282 | 271 | 282 | 22,200 | 100.0% | 73.3% |
| Amorphous sulfasalazine | 154 | 372 | 372 | 367 | 30,300 | 136.5% | 100.0% |
| 25% sulfasalazine:HPMCAS-MG | 34 | 725 | 571 | 725 | 44,400 | 200.0% | 146.5% |
| 50% sulfasalazine:PVP VA64 | 67 | 1,372 | 1,232 | 1,073 | 112,800 | 508.1% | 372.3% |
| 25% sulfasalazine:PVP VA64 | 425 | 2,350 | 2,319 | 2,290 | 196,200 | 883.8% | 647.5% |

### Example 10:Reformulation of Sulfasalazine Increases Oral Bioavailability In Vivo

The following experiments demonstrate that administration of a 25% sulfasalazine-TPGS:PVP VA64 SDD composition results in a significant increase in oral bioavailability compared to administration of crystalline sulfasalazine in a rat model.

### Preparation of Compounds:

Crystalline sulfasalazine was obtained from Sigma-Aldrich (St. Louis, Mo.), Catalog # S0883. Crystalline sulfasalazine was re-suspended in 0.5% Methocel (Methocel A4M Premium, Dow Chemical, Midland, Mich.) to a concentration of 40 mg/ml sulfasalazine. Re-suspension of the crystalline sulfasalazine composition was accomplished by adding the 0.5% Methocel drop-wise to the composition and mixing in a mortar and pestle until the composition were evenly resuspended to form the non-reformulated composition. Separately, a sample of 25% sulfasalazine:PVP VA64 SDD (Formulation Exemplar 2) was resuspended in 0.5% Methocel to a concentration of 40 mg/ml sulfasalazine per ml. The 25% sulfasalazine:PVP VA64 SDD composition was re-suspended by adding the 0.5% Methocel drop-wise to the composition and mixing in a mortar and pestle until the composition were evenly resuspended, forming the reformulated composition.

A 25% sulfasalazine-TPGS:PVP VA64 SDD composition and a 25% sulfasalazine-Tween 20:PVP VA64 SDD composition may be prepared and tested in a similar manner.

The pharmaceutical preparations are made following conventional techniques, including but not limited to milling, mixing, granulation, ballmilling, shaking, calendaring, tumbling, stirring or rollmilling, and compressing, when necessary, for tablet forms. For the preparation of hard gelatin capsule forms, the composition may be prepared by milling, mixing, granulation, ballmilling, shaking, calendaring, tumbling, stirring or rollmilling and filling. Other standard manufacturing procedures are described in Modern Plastics Encyclopedia, Vol 46, pp 62-70 (1969); and in Pharmaceutical Science, by Remington, 14th Ed, pp 1626-1678 (1970), published by Mack Publishing Co, Easton, Pa., and as described herein.

For example, a composition comprising sulfasalazine, such as the spray-dried dispersion (SDD) comprising a polymer, and TPGS may be prepared by mixing the prescribed amount of sulfasalazine with TPGS and milling the mixture by hand using a mortar and pestle, at medium pressure, for a period of time sufficient to prepare a fine powder or mixture. Depending on the amount of the composition being milled using a particular size mortar and pestle, hand milling may be performed for about 1 to 10 minutes, and the composition is noted for the level or amount of fine powder or fine mixture being formed. For larger sample preparation, such as with samples that is more than 1-2 grams, hand milling may be performed for 1-10 minutes, and repeated milling for an additional 1-10 minutes or more as needed to obtain a fine, homogeneous powder or mixture.

### Animal Study Design, Dosing and Plasma Collection:

A total of 6 Sprague-Dawley rats were used in the study, divided into 2 cohorts as shown in Table 10. All rats were males that ranged in weight from 202 grams to 214 grams apiece. Rats were allowed to eat ad libitum before testing. Independently, the crystalline sulfasalazine formulation and the reformulated 25% sulfasalazine:75% PVP VA64 SDD composition were administered by gastric lavage at a dose of 400 mg/kg. Following drug administration, 200 μl of plasma was collected from each animal at the following time points: 30, 60, 90, 120, 160 and 240 minutes. Plasma samples were snap frozen in liquid N₂ and stored at −80° C. until analysis.

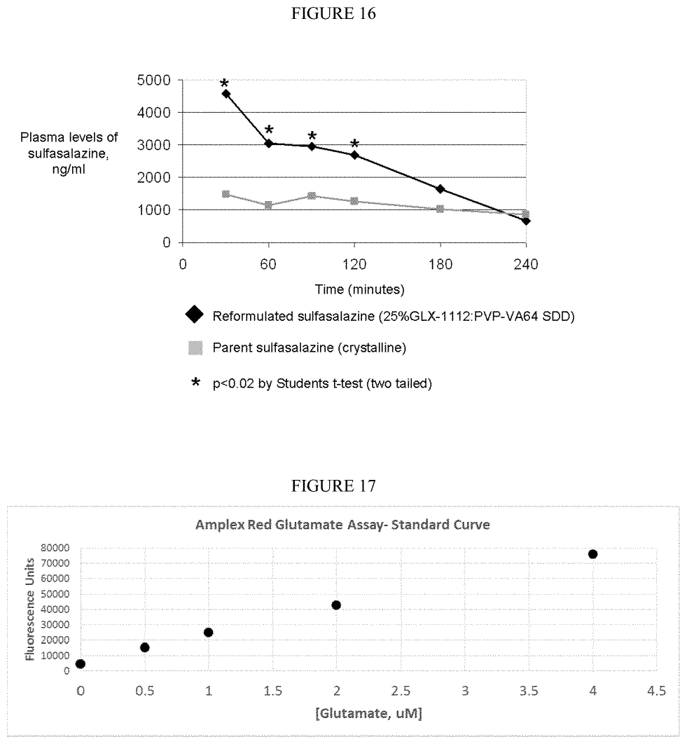

Levels of sulfasalazine detected in the rat plasma at the different time points are given in Table 11, the summary and statistical values are given in Table 12 and the data presented in graphical format in FIG. 16. One rat (#6326) showed evidence that drug was partially administered to the lungs, resulting in high plasma levels, and values from this rat were not included in calculating mean values or in the statistical analysis. Oral administration of sulfasalazine, both the crystalline and the 25% sulfasalazine:PVP VA64 SDD formulation showed immediate plasma accumulation within the first 30 minutes of administration. The reformulated sulfasalazine (25% sulfasalazine:PVP VA64 SDD) showed higher plasma levels, ranging from approximately 300% at the first 30 minute time point to about 160% at the 3 hour time point, when compared to the crystalline sulfasalazine composition following oral administration.

Similarly, a reformulated sulfasalazine (25% sulfasalazine-TPGS:PVP VA64 SDD) provides higher plasma levels, as noted above.

TABLE 11
Concentrations of sulfasalazine in plasma.

| | | Analyte | | | | | | | |
|---|---|---|---|---|---|---|---|---|---|
| | | Sulfasalazine Levels, Plasma (ng/ml) | | | | | | | |
| | | Treatment | | | | | | | |
| | | Refomulation (25% GLX-1112:PVP- | | | | | | | |
| | | VA64 SDD) | | | | Parent (Crystalline) | | | |
| | | Subject ID | | | | | | | |
| | | | | | Mean | | | | Mean |
| | | 6326* | 6327 | 6328 | Levels | 6329 | 6330 | 6331 | Levels |
| Time | 0.5 h | 23,200 | 4,760 | 4,370 | 4,565 | 1,390 | 1,270 | 1,800 | 1,487 |
| Points | 1 h | 27,400 | 3,020 | 3,080 | 3,050 | 1,050 | 1,380 | 991 | 1,140 |
| | 1.5 h | 17,100 | 2,980 | 2,910 | 2,945 | 1,340 | 1,810 | 1,130 | 1,427 |
| | 2 h | 10,400 | 2,720 | 2,660 | 2,690 | 1,050 | 1,380 | 1,340 | 1,257 |
| | 3 h | 7,130 | 1,520 | 1,780 | 1,650 | 939 | 787 | 1,360 | 1,029 |
| | 4 h | 3,790 | 271 | 1,050 | 661 | 872 | 790 | 887 | 850 |

*Test animal exhibited evidence that drug was partially administered to lung; values for this animal were ommitted from mean value calculation.

TABLE 12
Summary and statistics of bioavailability experiment. All
statistics were calculated using two-tailed Students t-test.

| | Reformulation | | | |
|---|---|---|---|---|
| | (25% GLX- | | | |
| | 1112:PVP- | Parent | Percent | |
| | VA64 SDD) | (Crystalline) | difference | |
| Time | Mean | Mean | Reformulated/ | p- |
| Points | Values | Values | Parent | value |
| 0.5 h | 4,565 | 1,487 | 307% | 0.0012 |
| 1 h | 3,050 | 1,140 | 267% | 0.0012 |
| 1.5 h | 2,945 | 1,427 | 206% | 0.0100 |
| 2 h | 2,690 | 1,257 | 214% | 0.0018 |
| 3 h | 1,650 | 1,029 | 160% | 0.0820 |
| 4 h | 651 | 850 | 78% | 0.7755 |

FIG. 16 shows the mean values of plasma sulfasalazine plotted in graphical format.

The results of the above experiments demonstrate that: (1) the reformulated sulfasalazine formulation attains higher plasma concentrations following oral administration than the crystalline formulation of sulfasalazine and that (2) the increase in plasma concentrations are approximately 300% percent to 160% over the first 3 hours of administration. These results demonstrate that sulfasalazine can be reformulated to increase oral bioavailability.

### Example 11: Monitoring xCT Levels in Primary Microglia in Response to Agents Known to Cause Activation of Neuroinflammatory Cells and/or to Cause or Reflect Damage to Neurons, Axons and/or Oligodendrocytes

The following experiments demonstrate that levels of xCT mRNA: (1) can be monitored in primary microglia by quantitative PCR (qPCR); (2) that levels of xCT increase in response to the presence of LPS, a stimulator of the innate immune system that acts through binding the ligand of the Toll-Like Receptor 4 (TLR4); (3) that induction of xCT gene expression by LPS occurs in different media and when normalized to different control genes; and (4) LPS also activates microglia as monitored by expression of IL1-beta, a well-known reporter gene for microglial activation.

Primary rat microglial cells were purchased from Lonza (Allendale, N.J.), thawed and resuspended in 1 ml of microglia media (88% DMEM (#12-604F, Lonza)/10% FBS (#F4135, Sigma-Aldrich), 100 units Penicillin-Streptomycin (#17-602E, Lonza), 4.5 g/L D-glucose (#G-8769, Sigma-Aldrich) and aliquoted to 11 wells in a 96-well plate. In this and all subsequent work, cells were maintained in an incubator at 37° C. with 5% CO₂. For each qPCR experiment, 1-3 wells (total of 153,000-459,000 live cells) were transferred to a new 96-well plate with fresh microglia media. Following 24 hours, cells were then mixed and aliquoted in to 96 wells at 7,000-20,000 cells per well in fresh media, either complete media (microglia) or minimal media (100 mL Neurobasal Media (#12348-017, Life Tech) supplemented with 1 mL N2 supplement (#17502-048, Life Tech) and 0.5 mM GlutaMax (#35050-061, Life Tech). Compounds to be tested were then added at the indicated concentrations and cells harvested 18 hours later by centrifugation at 2000×g, 5 minutes at 4° C. mRNA was isolated from the cells by use of the Purelink microscale RNA extraction kit (Catalog #12183016, Life Technologies) kit and the mRNA resuspended in 12-22 μl of RNase-free water according to the manufacturer's instructions. cDNA was prepared by use of the Superscript VILO kit (Catalog #11755050, Life Technologies) according to the manufacturer's instructions. The cDNA was eluted in a final volume of 20 μl RNase-free water and 4 μl was used for each qPCR reaction.

For the real-time qPCR reactions, primers and reagents were supplied by Life Technologies and used according to the manufacturer's instructions. The detection molecule was the TaqMan probe with FAM dye label on 5′ end that binds to non-labeled primer pairs. The qPCR machine was a model 7500 Fast Real Time machine from ABI and used according to the manufacturer's instructions. 4 μl of the cDNA reaction was used for each qPCR reactions in a final volume of 20 μl. Primers were added to a concentration of 1 μM. qPCR cycling times were as follows: 50° C. for 2 min, 95° C. for 10 min, 95° C. for 15 sec, 60° C. for 1 min for a total of 40 cycles.

mRNA levels were quantitated by measuring the Cycle threshold (Ct) for each qPCR reaction using the standard algorithm supplied with the ABI 7500 Fast qPCR machine without modification. The Ct is defined as the number of cycles required for the fluorescent signal to cross the threshold (e.g. exceeds background level). Ct levels are inversely proportional to the amount of target nucleic acid in the sample (e.g. the lower the Ct level the greater the amount of target nucleic acid in the sample. The change in Ct values under different conditions is logarithmically proportional to the changes in mRNA levels by the formula: Change in mRNA level=2\hat{ }^{(Change in Ct values)}. Expression of xCT and other genes of interest was first normalized by changes in Ct values of the control genes. The control genes used in these experiments were the gamma-actin gene and/or the HPRT gene, both of which are well known control genes for qPCR experiments.

The qPCR primers used in the experiments were standard primers recommended for each gene of interest by Life Technologies. Table 13 lists the genes and catalog numbers of the primers used in the quantitative PCR (qPCR) reactions.

TABLE 13
qPCR primer information

| Gene | Catalog number |
|---|---|
| xCT | 4331182 |
| Gamma actin | 4351372 |
| HPRT | 4331182 |
| IL-1 beta | 4331182 |
| Lcn-2 | 4331182 |

In these and the following experiments, the microglia or astrocytes were exposed to various agents. With the exception of IL-4, all the agents tested had previously been known to cause activation of neuroinflammatory cells and/or known to cause or reflect damage to neurons, axons and/or oligodendrocytes. In contrast, IL4 is an anti-inflammatory cytokine and was tested as a control. Table 14 lists the type of stimulus, the test agents and the sources for the agents tested in the primary microglial and/or astrocyte cell cultures.

TABLE 14
Agents tested in primary microglial and/or astrocyte cell cultures.

| Type of Stimulus | Agent | Source |
|---|---|---|
| Innate Immune system-Toll Like | LPS-ultrapure from E. | Catalog #L4391; Sigma-Aldrich (St. |
| Receptor 4 | coli 011:B4 | Louis, MO) |
| Innate Immune system-Toll Like | Poly I/C | Catalog #tlrl-pic; InVivoGen (San |
| Receptor 3 | | Diego, CA) |
| Innate Immune system-Toll Like | Pam2CSK4b (Synthetic | Catalog #tlrl-pm2s-1; InVivoGen |
| Receptor 2 | diacylated lipoprotein) | (San Diego, CA) |
| Misfolded/aggregated proteins | A-beta 1-40 peptide | Catalog #H-1194.0100; Bachem |
| | | (Bubendorf, Switzerland) |
| Misfolded protein | Tunicamycin | Catalog #T7765; Sigma-Aldrich (St. |
| response/Endoplasmic reticulum | | Louis, MO) |
| stress response | | |
| Reactive Oxygen Species | Hydrogen Peroxide | Walgreens, 3% solution (San Carlos, |
| | (H₂O₂) | CA) |
| Inflammatory cytokine | TNF-alpha | Catalog #400-14, Preprotech (Rocky |
| | | Hill, NJ) |
| Innate immune system-DAMP | H1b (Histone H1b) | Catalog #M25015; New England |
| (Damage-associated molecular | | Biolabs (Ipswich, MA) |
| pattern molecule) | | |
| Inflammatory cytokine | IFN-gamma | Catalog #I3275; Sigma-Aldrich (St. |
| | | Louis, MO) |
| Inflammatory cytokine | IL-1-beta | Catalog #400-01B, Preprotech |
| | | (Rocky Hill, NJ) |
| Inflammatory cytokine | IL-6 | Catalog #I0406; Sigma-Aldrich (St. |
| | | Louis, MO) |
| Inflammatory cytokine | IL-17A | Catalog #8410-IL-25; R&D Systems |
| | | (Minneapolis, MN) |
| Anti-inflammatory cytokine | IL-4 | Catalog #I3650; Sigma-Aldrich (St. |
| | | Louis, MO) |

Previous work has demonstrated that the TLR4 ligand, LPS, induces xCT protein expression in primary rat microglia, as detected by Western blotting, e.g. Domercq et al 2007. To determine if LPS increased levels of xCT mRNA, LPS (100 ng/ml) was added to the primary microglial cell cultures in either complete or minimal media. xCT gene expression was determined by qPCR after 18 hours of exposure to LPS.

Table 15 provides the data for the initial qPCR reactions in primary microglia. The Cycle threshold is abbreviated Ct. The change in Ct equals [Ct (minus test agent)−Ct (plus test agent)]. Changes in Ct of the test genes xCT and IL1-beta are normalized by subtracting the change in Ct of the control gene, either gamma-actin or Hypoxanthine-guanine phosphoribosyl transferase (HPRT). Ct values are converted to absolute values by the formula: absolute value=2\hat{ }^{(normalized change in Ct)}. Absolute values are converted to percent change by multiplying the absolute value by 100.

As noted in Table 15, LPS greatly increased xCT expression in both complete and minimal media. xCT expression was increased by over 7,000% regardless of the media conditions or whether normalized to gamma-actin or normalized to HPRT expression as detected by qPCR analysis.

TABLE 15
LPS induces xCT gene expression in primary microglial in both complete media
and in minimal media.

| | Ct values | | | |
|---|---|---|---|---|
| Condition | xCT | IL1-beta | Gamma actin | HPRT |
| Complete media | | | | |
| Ct with no LPS | 35.923 | 40.000 | 34.307 | 34.272 |
| Ct with LPS, 100 ng/ml | 27.253 | 27.369 | 32.524 | 32.122 |
| Change in Ct | 8.670 | 12.631 | 1.783 | 2.15 |
| Change in xCT or IL1-beta Ct normalized | 6.887 | 10.848 | n/a | n/a |
| to Gamma actin | | | | |
| Absolute change in xCT or IL1-beta | 118.4 | 1,843.20 | n/a | n/a |
| normalized to Gamma-actin | | | | |
| Complete media | | | | |
| Percent change in xCT or IL1-beta | 11,836% | 184,320% | n/a | n/a |
| normalized to Gamma-actin | | | | |
| Change in xCT or IL1-beta Ct normalized | 6.520 | 10.481 | n/a | n/a |
| to HPRT | | | | |
| Absolute change in xCT or IL1-beta | 91.8 | 1,429.21 | n/a | n/a |
| normalized to HPRT | | | | |
| Percent change in xCT or IL1-beta | 9177% | 142,921% | n/a | n/a |
| normalized to HPRT | | | | |
| Minimal media | | | | |
| Ct with no LPS | 37.540 | 36.664 | 36.851 | 35.141 |
| Ct with LPS, 100 ng/ml | 25.746 | 21.838 | 31.210 | 32.403 |
| Change in Ct | 11.794 | 14.826 | 5.641 | 2.738 |
| Change in xCT Ct normalized to Gamma | 6.153 | 9.185 | n/a | n/a |
| actin | | | | |
| Absolute change in xCT normalized to | 71.2 | 582.14 | n/a | n/a |
| Gamma-actin | | | | |
| Percent change in xCT normalized to | 7,116% | 58,214% | n/a | n/a |
| Gamma-actin | | | | |
| Change in xCT Ct normalized to HPRT | 9.056 | 12.088 | n/a | n/a |
| Absolute change in xCT | 532.3 | 4355.3 | n/a | n/a |
| normalized to HPRT | | | | |
| Percent change in xCT normalized to | 53,226% | 435,530% | n/a | n/a |
| HPRT | | | | |

n/a = not applicable

The experiment shown in Table 15 demonstrates that: (1) qPCR reliably detects changes in xCT and IL1-beta expression; (2) that the TLR4 ligand, LPS, activates microglia as shown by an significant increase in IL1-beta expression; (3) xCT expression is also highly induced by LPS; (4) both gamma-actin and HPRT can serve as appropriate normalization controls in the qPCR reaction; and (5) the increase in xCT expression is robust and not significantly affected by the type of media used to incubate the cells.

### Example 12: Levels of xCT Increase in Primary Microglia in Response to Agents Known to Cause Activation of Neuroinflammatory Cells and/or to Cause or Reflect Damage to Neurons, Axons and/or Oligodendrocytes

Using the methods described in Example 11, various agents reported to cause activation of neuroinflammatory cells and/or known to cause or reflect damage to neurons, axons and/or oligodendrocytes were tested for their effect on xCT levels in primary rat microglia.

Table 16 provides the changes in mRNA levels of xCT in response to the various test agents in primary microglia. The type of test agent, the identity and concentration of the test agent, the type of media and the identity of the control gene are given. For xCT, the Ct values are given, as is the change in absolute value and percentage. For IL1-beta, only the percentage value is given.

TABLE 16
Changes in mRNA levels of xCT in response to various test agents

| | | | | | | | IL1- |
|---|---|---|---|---|---|---|---|
| | | | | xCT, | xCT, | xCT, | beta, |
| Type of | | | Control | Change | Fold | Percent | Percent |
| Stimulus | Agent | Media | gene | in Ct* | change* | change* | change* |
| Innate immune | Poly I/C, | Complete | Gamma- | 6.186 | 72.8 | 7,281% | 1,900% |
| system-TLR3 | 100 ng/ml | media | actin | | | | |
| Ligand | | | | | | | |
| Innate immune | Poly I/C, | Minimal | Gamma- | 3.619 | 12.3 | 1229% | 1,099% |
| system-TLR3 | 100 ng/ml | media | actin | | | | |
| Ligand | | | | | | | |
| Inflammatory | TNF-alpha, | Complete | Gamma- | 3.214 | 9.3 | 930% | 3,000% |
| cytokine | 100 ng/ml | media | actin | | | | |
| Inflammatory | IFN-gamma, | Complete | Gamma- | 1.417 | 2.7 | 270% | 2,043% |
| cytokine | 30 ng/ml | media | actin | | | | |
| Inflammatory | IFN-gamma, | Complete | Gamma- | 1.636 | 3.1 | 311% | 446% |
| cytokine | 100 ng/ml | media | actin | | | | |
| Inflammatory | TNF-alpha, | Complete | Gamma- | 3.562 | 11.8 | 1,181% | 11,395% |
| cytokine | 100 ng/ml + | media | actin | | | | |
| | IFN-gamma, | | | | | | |
| | 30 ng/ml | | | | | | |
| Inflammatory | IL-17A, 10 | Complete | Gamma- | 1.652 | 3.1 | 314% | n/d |
| cytokine | ng/ml | media | actin | | | | |
| Innate immune | Histone | Minimal | Gamma- | 2.364 | 5.1 | 515% | 30% |
| system-DAMP | H1b, 4 | media | actin | | | | |
| ligand | ug/ml | | | | | | |
| Anti- | IL-4, 100 | Complete | Gamma- | −0.147 | 0.9 | 90% | n/d |
| inflammatory | ng/ml | media | actin | | | | |
| cytokine | | | | | | | |

*normalized to changes in control gene
n/d = not determined

The experiments summarized in Table 16 demonstrate that: (1) xCT expression is significantly induced by a variety of agents that are known to cause activation of neuroinflammatory cells and/or to cause or reflect damage to neurons, axons and/or oligodendrocytes, including stimulators of the innate immune system, such as the TLR3 ligand poly I/C and the Damage-associated molecular pattern pathway ligand H1b, and inflammatory cytokines, such as TNF-alpha, IFN-gamma and IL-17A; (2) in almost all cases, an increase in xCT levels was accompanied by an increase in IL1-beta levels, consistent with the agent being tested also causing a classical activation phenotype of the microglia. One agent, the Damage-associated molecular pattern pathway ligand H1b, increased xCT levels but did not cause an increase in IL1-beta levels. This suggests that xCT expression may be more responsive to a variety of neuroinflammatory and/or neurodegenerative agents than IL1-beta expression; and (3) the anti-inflammatory cytokine IL-4 did not cause an increase in xCT levels. The expression profile of xCT in primary microglia is consistent with a neuroinflammatory and/or neurodegeneration phenotype, with xCT expression increasing in response to agents known to cause activation of neuroinflammatory cells and/or cause or reflect stress and damage to neurons, axons and/or oligodendrocytes.

### Example 13: Monitoring xCT Levels in Primary Astrocytes in Response to Agents Known to Cause Activation of Neuroinflammatory Cells and/or to Cause or Reflect Damage to Neurons, Axons and/or Oligodendrocytes

The following experiments demonstrate that levels of xCT mRNA: (1) can be monitored in primary astrocytes by quantitative PCR (qPCR), (2) that levels of xCT increase in response to the presence of tunicamycin, a stimulator of endoplasmic stress that acts through the unfolded protein response pathway, e.g. Schinthal, 2012, (3) that induction of xCT gene expression by tunicamycin occurs when normalized to different control genes and (4) tunicamycin also activates microglia as monitored by expression of Lcn-2, a reporter gene for astrocyte activation.

Primary rat astrocyte cells were purchased from All Cells (Catalog #RCTX-001F, Alameda, Calif.), thawed and resuspended in 13.4 ml of DMEM media (Catalog #12-614F, Lonza, Allendale, N.J.) supplemented with 15% v/v FBS (Catalog #F4135, Sigma-Aldrich, St. Louis, Mo.) and 560 μL aliquoted into wells in a 24-well plate. Cells were incubated at 37° C. with 5% CO₂. For each qPCR experiment, 2-3 wells (approximately 42,000-63,000 cells) were transferred to a new 24-well plate with fresh astrocyte media, composed of 50% v/v DMEM, 50% v/v Neurobasal media (Catalog #12348-017), Life Technologies, supplemented with 2 mM glutamine, 300 μM L-cystine. Following 24 hours, cells were then mixed and aliquoted into 24 well plates at approximately 10,000 cells per well in the same media. Compounds to be tested were then added at the indicated concentrations and cells harvested 18 hours later by washing once with PBS (Catalog #P5493, Sigma-Aldrich) and then treated with 300 μl of StemProAccutase cell dissociation reagent (Catalog #A11105-01, Life Technologies) and incubated for 5-10 minutes at 37° C. PBS (300 μl) was added and the cells spun down in a microfuge for 5 minutes at 2,000×g at 4° C. The supernatant was removed and mRNA was isolated from the cells by using the Purelink microscale RNA extraction kit (Catalog #12183016, Life Technologies) according to the manufacturer's instructions. The mRNA resuspended in 12-22 μl of RNase-free water. cDNA was prepared by use of the Superscript VILO kit (Catalog #11755050, Life Technologies) according to the manufacturer's instructions. The cDNA was eluted in a final volume of 20 μl RNase-free water in and 4 μl was used for each qPCR reaction. qPCR reactions and primers were as described in Example 11.

Previous work has demonstrated that tunicamycin induces xCT protein expression in immortalized rat fibroblasts, as detected by Northern blotting, e.g. Sato et al. 2004. To determine if tunicamycin increased levels of xCT mRNA, Tunicamycin (5 ng/ml) was added to the primary astrocyte cell cultures in astrocyte media. xCT gene expression was determined by qPCR after 18 hours of exposure to tunicamycin.

Table 17 provides the data for the initial qPCR reactions in primary astrocytes. The Cycle threshold is abbreviated Ct. The change in Ct equals [Ct (minus test agent)−Ct (plus test agent)]. Changes in Ct of the test genes xCT and Lcn-2 are normalized by subtracting the change in Ct of the control gene, either gamma-actin or Hypoxanthine-guanine phosphoribosyl transferase (HPRT). Ct values are converted to absolute values by the formula: absolute value=2\hat{ }^{(normalized change in Ct)}. Absolute values are converted to percent change by multiplying the absolute value by 100.

Table 17 shows tunicamycin greatly increased xCT expression as detected by qPCR. Importantly, xCT expression was increased by over 2,000% regardless of whether normalized to gamma-actin or normalized to HPRT expression.

TABLE 17
Tunicamycin induces xCT gene expression in primary astrocytes

| Condition | xCT | Lcn-2 | Gamma actin | HPRT |
|---|---|---|---|---|
| Ct with no tunicamycin | 30.589 | 32.857 | 28.960 | 28.905 |
| Ct with tunicamycin, 5 ng/ml | 25.060 | 27.123 | 29.240 | 28.906 |
| Change in Ct | 5.529 | 5.734 | −0.28 | 0.999 |
| Change in xCT or Lcn-2 Ct normalized | 5.809 | 6.014 | n/a | n/a |
| to Gamma actin | | | | |
| Absolute change in xCT or Lcn-2 | 56.1 | 64.6 | n/a | n/a |
| normalized to Gamma-actin | | | | |
| Percent change in xCT or Lcn-2 | 5,606% | 6,460% | n/a | n/a |
| normalized to Gamma-actin | | | | |
| Change in xCT or Lcn-2 Ct normalized | 4.530 | 5.015 | n/a | n/a |
| to HPRT | | | | |
| Absolute change in xCT or Lcn-2 | 23.1 | 32.3 | n/a | n/a |
| normalized to HPRT | | | | |
| Percent change in xCT or Lcn-2 | 2,310% | 3,230% | n/a | n/a |
| normalized to HPRT | | | | |

n/a = not applicable

The results shown in Table 17 demonstrate that: (1) qPCR reliably detects changes in xCT and Lcn-2 expression in primary astrocytes; (2) that an agent that induces the unfolded protein response activates astrocytes as demonstrated by a significant increase in Lcn-2 expression; (3) xCT expression is also highly induced by Tunicamycin in astrocytes; and (4) both gamma-actin and HPRT can serve as appropriate normalization controls in the qPCR reaction.

### Example 14: Levels of xCT Increase in Primary Astrocytes in Response to Agents Known to Cause Activation of Neuroinflammatory Cells and/or to Cause or Reflect Damage to Neurons, Axons and/or Oligodendrocytes

Using the methods described in Example 11, various agents known to cause activation of neuroinflammatory cells and/or cause or reflect stress and damage to neurons, axons and/or oligodendrocytes were tested for causing an increase in xCT levels in primary rat astrocytes.

Table 18 provides the changes in mRNA levels of xCT in response to various test agents in primary astrocytes. The type of test agent, the identity and concentration of the test agent, the type of media and the identity of the control gene are given. For xCT, the Ct values are given, as is the change in absolute value and percentage. For Lcn-2, only the percentage value is given.

TABLE 18
Changes in mRNA levels of xCT in response to various test agents

| | | | | xCT, | xCT, | xCT, | Lcn-2, |
|---|---|---|---|---|---|---|---|
| Type of | | | Control | Change | Fold | Percent | Percent |
| Stimulus | Agent | Media | gene | in Ct* | change* | change* | change* |
| Innate | LPS, 100 | Astrocyte | Gamma- | 2.078 | 4.22 | 422% | 692% |
| immune | ng/ml | media | actin | | | | |
| system- | | | | | | | |
| TLR4 Ligand | | | | | | | |
| Innate | LPS, 100 | Astrocyte | HPRT | 1.181 | 2.27 | 227% | n/d |
| immune | ng/ml | media | | | | | |
| system- | | | | | | | |
| TLR4 Ligand | | | | | | | |
| Innate | LPS, 100 | Astrocyte | Gamma- | 2.71 | 6.54 | 654% | 1579% |
| immune | ng/ml | media + | actin | | | | |
| system- | | 2.5% | | | | | |
| TLR4 Ligand | | fetal | | | | | |
| | | bovine | | | | | |
| | | serum | | | | | |
| Innate | Poly I/C, 10 | Astrocyte | Gamma- | 1.471 | 2.77 | 277% | 76% |
| immune | ug/ml | media | actin | | | | |
| system- | | | | | | | |
| TLR3 Ligand | | | | | | | |
| Innate | Pam2CSK4, | Astrocyte | Gamma- | 0.65 | 1.57 | 157% | 1,602% |
| immune | 200 ng/ml | media | actin | | | | |
| system- | | | | | | | |
| TLR2 Ligand | | | | | | | |
| Unfolded | Tunicamycin, | Astrocyte | Gamma- | 5.915 | 60.34 | 6,034% | 5,940% |
| Protein | 25 ng/ml | media | actin | | | | |
| response | | | | | | | |
| Unfolded | Tunicamycin, | Astrocyte | HPRT | 4.540 | 23.26 | 2,326% | 2,290% |
| protein | 25 ng/ml | media | | | | | |
| response | | | | | | | |
| Misfolded | A-beta 1-40, | Astrocyte | Gamma- | 0.276 | 1.21 | 121% | 130% |
| protein | 2 μg/ml | media | actin | | | | |
| Misfolded | A-beta 1-40, | Astrocyte | Gamma- | 0.951 | 1.93 | 193% | 140% |
| protein | 20 ug/ml | media | actin | | | | |
| Reactive | H₂O₂, 5 μM | Astrocyte | Gamma- | 2.484 | 5.59 | 559% | 15% |
| oxygen | | media | actin | | | | |
| species | | | | | | | |
| Reactive | H₂O₂, 50 μM | Astrocyte | Gamma- | 2.588 | 6.01 | 601% | 19% |
| oxygen | | media | actin | | | | |
| species | | | | | | | |
| Inflammatory | TNF-alpha, | Astrocyte | Gamma- | 1.376 | 2.60 | 260% | 244% |
| cytokine | 100 ng/ml | media | actin | | | | |
| Inflammatory | IL1-beta, 1 | Astrocyte | Gamma- | 1.538 | 2.90 | 290% | 23,522% |
| cytokine | ng/ml | media | actin | | | | |
| Inflammatory | IL1-beta, 1 | Astrocyte | HPRT | 1.536 | 2.90 | 290% | 23,480% |
| cytokine | ng/ml | media | | | | | |
| Inflammatory | IL1-beta, 100 | Astrocyte | Gamma- | 2.560 | 5.90 | 590% | 18,958% |
| cytokine | ng/ml | media | actin | | | | |
| Inflammatory | IL1-beta, 100 | Astrocyte | HPRT | 1.515 | 2.86 | 286% | 9,185% |
| cytokine | ng/ml | media | | | | | |
| Inflammatory | IL-6, 20 | Astrocyte | Gamma- | 0.366 | 1.29 | 129% | 146% |
| cytokine | ng/ml | media | actin | | | | |
| Inflammatory | IL-6, 100 | Astrocyte | Gamma- | 0.380 | 1.30 | 130% | 130% |
| cytokine | ng/ml | media | actin | | | | |
| Inflammatory | IL-17A, 100 | Astrocyte | Gamma- | 0.351 | 1.28 | 128% | 123% |
| cytokine | ng/ml | media | actin | | | | |
| Anti- | IL-4, 100 | Astrocyte | Gamma- | −0.097 | 0.93 | 93% | 103% |
| inflammatory | ng/ml | media | actin | | | | |
| cytokine | | | | | | | |

*normalized to changes in control gene
n/d = not determined

The experiments summarized in Table 18 demonstrate that: (1) xCT expression is significantly induced by a variety of agents that are known to cause activation of neuroinflammatory cells and/or to cause or reflect damage to neurons, axons and/or oligodendrocytes, including stimulators of the innate immune system, such as the TLR4 ligand, LPS, the TLR3 ligand poly I/C and the TLR2 ligand Pam2CSK; inflammatory cytokines, such as TNF-alpha, IL1-beta, IL-6 and IL-17A; reactive oxygen species, such as hydrogen peroxide; and inducers of ER stress, such as the unfolded protein response inducer, Tunicamycin; (2) in almost all cases, an increase in xCT levels was accompanied by an increase in Lcn-2 levels, consistent with the agent being tested also causing a classical activation phenotype of the astrocytes. Several agents, including the TLR3 ligand poly I/C and the reactive oxygen species, hydrogen peroxide, increased xCT levels but did not cause an increase in Lcn-2 levels. This suggests that xCT expression may, in some cases, be more responsive to neuroinflammatory and/or neurodegenerative agents than Lcn-2 expression; and (3) the anti-inflammatory cytokine IL-4 did not cause an increase in xCT levels. The expression profile of xCT in primary astrocytes is consistent with a neuroinflammatory and/or neurodegeneration phenotype, with xCT expression increasing in response to agents known to cause activation of neuroinflammatory cells and/or cause or reflect stress and damage to neurons, axons and/or oligodendrocytes.

### Example 15: Agent that Increases xCT Levels Also Increases Functional Protein Activity of xCT

Previous work described in Examples 11-14 demonstrated that xCT mRNA expression levels are increased by a variety of agents implicated in neuroinflammation and/or neurodegeneration. To test whether this increase in mRNA expression resulted in increased levels of functional protein, as reflected by an increase in extracellular glutamate, xCT protein activity in primary astrocytes was measured following exposure to TNF-alpha, an inflammatory cytokine that increased xCT mRNA levels by 260% in previous qPCR testing (Example 14).

xCT is a cystine-glutamate exchange protein that imports extracellular cystine in exchange for exporting intracellular glutamate. xCT activity can be measured by measuring the level of the extracellular glutamate that is exported. For detection of extracellular glutamate, the Amplex Red Glutamic Acid/Glutamate Oxidase Assay Kit from Life Technologies (Catalog #A-1222, Grand Island, N.Y.) was used following the manufacturer's instructions. Fluorescence from the Amplex Red fluorophore is directly proportional to glutamate concentration and was detected on an Enspire plate reader (Perkin-Elmer (Model #2300, Santa Clara, Calif.) with excitation wavelength at 530 nM and detection wavelength at 590 nM according to the manufacturer's instructions.

Astrocytes were grown in 24-well microtiter plates at a density of approximately 10,000 cells/well in a total volume of 400 μl of astrocyte media. Cells were incubated 18 hours in either astrocyte media plus 100 μM cystine (Catalog #C8755; Sigma-Aldrich) or astrocyte media plus 100 μM cystine plus TNF-alpha (100 μg/ml). After 18 hours, the media was removed and the adherent astrocytes washed twice with PBS and then 400 μL of minimal media was added to the cells. The minimal media was EBSS (catalog #E3024, Sigma-Aldrich) supplemented with 100 μM cystine and 10 mM D-glucose (catalog #G8769, Sigma-Aldrich). For select cultures, sulfasalazine (catalog #S0883, Sigma-Aldrich), a specific inhibitor of glutamate release by xCT, was added to 50 μM concentration.

At 30, 120 and 240 minutes after change to minimal media (+/−sulfasalazine), 50 μL aliquots of the media were withdrawn from the wells and centrifuged for 10 minutes at 2,000×g at 4° C. to remove any cells or cellular debris. The top 25 μL of the supernatant was then transferred to a 0.5 ml microfuge tube and the aliquots frozen at −80.

For the glutamate assay, samples of the media were thawed, pipetted up and down to ensure mixing and 20 μl added to 80 μl of the Amplex Red assay mix in a 96-well microtiter plate (catalog #M4436, Greiner, Frickenhausen, Germany). The reactions were covered in aluminum foil and allowed to proceed for 30 minutes at 37° C. and then fluorescence measured as described above.

Concurrently with analysis of the astrocyte samples, a standard curve of the Amplex Red glutamate assay in the minimal media was prepared following the manufacturer's instructions and using known concentrations of glutamate: 0, 0.5, 1, 2 and 4 μM. Table 19 gives the numerical results of the standard curve assay and the results are depicted graphically in FIG. 17. The data show (1) in the absence of the Amplex red fluorophore, there is no fluorescence from any other components of the assay (row 2); and (2) that the fluorescence from the complete Amplex Red Glutamic Acid/Glutamate Oxidase Assay is linearly proportional to glutamate concentration. Linear regression analysis of the standard curve data gives a high correlation coefficient of 0.9995, demonstrating that the assay can quantitate glutamate concentrations with high precision.

TABLE 19
Standard curve values for the Amplex Red Glutamic
Acid/Glutamate Oxidase Assay

| Complete Amplex Red | Final concentration of | Fluorescence |
|---|---|---|
| assay reagents? | glutamate, actual (μM) | units |
| No-No Amplex | 0 | 53 |
| Red fluorophore | | |
| Yes | 0 | 4,472 |
| Yes | 0.5 | 15,194 |
| Yes | 1.0 | 25,010 |
| Yes | 2.0 | 42,699 |
| Yes | 4.0 | 75,739 |

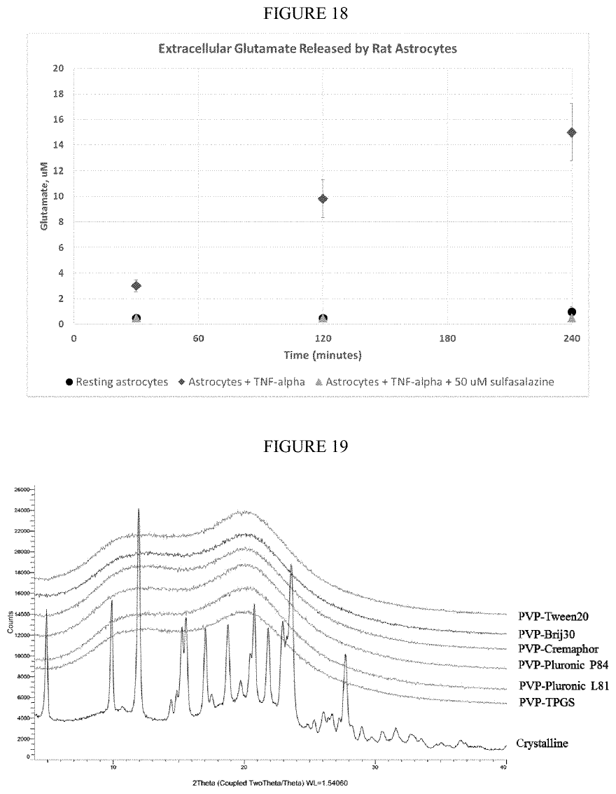

Using the same Amplex Red Glutamic Acid/Glutamate Oxidase Assay the experimental samples from the minimal media were analyzed. Table 20 gives the numerical results of the assay of the samples from the minimal media and the results are depicted graphically in FIG. 18.

TABLE 20
Experimental values from samples taken from the minimal media using the Amplex
Red Glutamic Acid/Glutamate Oxidase Assay

| Additions to astrocyte and/or minimal media | | | | | |
|---|---|---|---|---|---|
| Cystine (both | TNF-alpha | Sulfasalazine | | | Calculated |
| astrocyte and | (astrocyte | (minimal media | Time | Fluorescence | glutamate |
| minimal media) | media only) | only) | (minutes) | units | levels (μM)* |
| 100 μM | No TNF-alpha | No | 30 | 4459 | 0.25 |
| 100 μM | No TNF-alpha | No | 120 | 4487 | 0.26 |
| 100 μM | No TNF-alpha | No | 240 | 6209 | 0.77 |
| 100 μM | 100 μg/ml | No | 30 | 13398 | 2.88 |
| 100 μM | 100 μg/ml | No | 120 | 34307 | 9.05 |
| 100 μM | 100 μg/ml | No | 240 | 50468 | 13.82 |
| 100 μM | 100 μg/ml | 50 μM | 30 | 4467 | 0.25 |
| 100 μM | 100 μg/ml | 50 μM | 120 | 4199 | 0.17 |
| 100 μM | 100 μg/ml | 50 μM | 240 | 4525 | 0.27 |

*Calculated using this formula: Glutamate concentration (μM) = [(Fluorescence units − 3,607.9)/16,958.2] * Dilution factor (e.g. the dilution factor; 20 μL, of minimal media into 100 μL, final volume of assay mix)

The data show (1) exposure of primary astrocytes to an inflammatory cytokine, TNF-alpha, that is known to increase xCT mRNA levels, also causes an increase in extracellular glutamate as detected by the Amplex Red assay; and (2) that the increase in extracellular glutamate levels can be blocked by specific inhibitors of xCT, such as sulfasalazine, demonstrating that this increase in glutamate is directly caused by functional xCT protein activity.

These results collectively demonstrate that: (1) A wide variety of agents known to cause or reflect damage to neurons, axons and oligodendrocytes, cause an increase in xCT expression, and (2) the increased expression of xCT causes release of glutamate into the extracellular space, where the glutamate is available to cause inappropriate activation of glutamate receptors and excitotoxicity.

### Example 16: Sulfasalazine Treatment Prevents Demyelination of the Optic Nerve in an Optic Neuritis Model

Optic neuritis is an inflammation of the optic nerve, the bundle of nerve fibers that transmits visual information from the eye to the brain. Pain and temporary vision loss are common symptoms of optic neuritis. Optic neuritis can occur with or without other symptoms of multiple sclerosis. Optic neuritis is of interest because the disease pathology includes components common in most demyelinating diseases, including demyelination and axon degeneration.

To determine the efficacy of sulfasalazine in optic neuritis, we used the 2D2-TCR MOG (“2D2”) mouse model developed by Bettelli et al and subsequently developed by Guan et al. When subject to a minimal autoimmune insult (e.g. low dose pertussis toxin or MOG peptide), approximately 80% of the 2D2 animals develop optic neuritis as determined by eye examination, pattern electroretinogram, MRI, OCT and histopathology, e.g. Talla et al, 2013 and Lidster et al, 2013. This model serves as an inducible model of optic neuritis with a high rate of penetrance. The development of optic neuritis was monitored by animal observation and histopathology.

### Animal Testing

Female 2D2 (strain C57BL/6-Tg(Tcra2D2,Tcrb2D2)1Kuch/J) and WT mice (C57BL/6) were purchased from The Jackson Laboratory. Each cohort size was 14 animals. Mice were delivered to Ophthy-DS (Kalamazoo, Mich.) under the supervision of Renovo Neural (Cleveland, Ohio) at 8-12 weeks of age and then acclimated for an additional 4 weeks before beginning the experiment. Animals were housed in pathogen-free, individually-ventilated, enriched cages on a 12 h:12 h light:dark cycle with food and water ad libitum. Mice were monitored daily for welfare and symptoms of optic neuritis.

After 4 weeks of acclimation, animals were randomized by weight into 4 groups of 14 animals each. To induce optic neuritis, 200 ng of Pertussis toxin was injected into the 2D2 animals on days 1 and 3 as described by Guan et al. Dosing of vehicle or treatment compounds began at day 1. The four cohorts are:
(1) WT mice (C57BL/6) treated with vehicle (200 μl saline IP, BID). This cohort serves as a control for the readouts of optic neuritis disease.
(2) 2D2 mice vehicle treated with vehicle (200 μl saline IP, BID). This cohort serves as a control for the treatment groups.
(3) 2D2 mice vehicle treated with sulfasalazine (200 mg/kg final dose in 200 μl saline IP, BID). This dose of sulfasalazine was previously shown to be efficacious in the SOD1 model of ALS; in particular, it increased survival time and decreased the numbers of activated microglia and astrocytes in the spinal cord.
(4) 2D2 mice vehicle treated with memantine (5 mg/kg final dose in 200 μl saline IP, BID). Memantine inhibits the NMDA glutamatergic receptor and has previously been shown to have efficacy in models of optic neuritis (Suhs, et al. 2014) and to reduce thinning of the RNFL in a clinical trial of optic neuritis (Esfahani, et al 2012). The memantine-treated group served as a control for anti-glutamatergic therapy in the 2D2 model of optic neuritis.

On day 30, animals were euthanized and perfused. Optic nerves and retinas were dissected out and embedded in paraffin and sections mounted on slides. 5-7 sections of the optic nerve (from one eye) from ten animals in each cohort were stained by Toluidine Blue. Histopathological analysis of the slides and quantitation of the myelin staining intensity was performed blinded by a board-certified pathologist, Dr. Igor Polyakov (Minneapolis, Minn.). Staining intensity of each slide was graded as either low, moderate or high. Both peripheral and central sections of the optic nerve were graded. The intensity of the myelin staining between the cohorts on a per section and a per animal basis was compared using chi-square and Fishers' exact test with a p<0.05 considered significant.

### Study Results

The staining in the peripheral and central sections of the optic nerves were highly correlated and thus these values were both used to give a single score for each animal or each section. On a per animal basis, this yielded 20 scores per cohort: average score per section×2 scores per animal (peripheral or central)×10 animals per cohort. On a per section basis, this yielded approximately 110 score per cohort: score per section×5-7 sections per animal×2 scores per section (peripheral or central)×10 animals per cohort. Table 21 contains the scores from this analysis and Table 22 summarizes the data and the statistical significance.

TABLE 21
Histopathology Scores

| | Number of animals-average myelin content score | | | |
|---|---|---|---|---|
| Per animal basis | WT | 2D2-vehicle | 2D2-memantine | 2D2-sulfasalazine |
| Myelin content-Central Region | | | | |
| Low | 0 | 0 | 0 | 0 |
| moderate | 3 | 10 | 7 | 6 |
| high | 7 | 0 | 3 | 4 |
| Myelin content-Peripheral Region | | | | |
| Low | 1 | 4 | 2 | 1 |
| moderate | 5 | 5 | 5 | 5 |
| high | 4 | 1 | 3 | 4 |
| Myelin content-Total | | | | |
| Low | 1 | 4 | 2 | 1 |
| Moderate | 8 | 15 | 12 | 11 |
| High | 11 | 1 | 6 | 8 |
| Sum | 20 | 20 | 20 | 20 |
| | Number of sections-average myelin content per section | | | |
| Per section basis | WT | 2D2-vehicle | 2D2-memantine | 2D2-sulfasalazine |
| Myelin content-Central Region | | | | |
| Low | 0 | 0 | 0 | 0 |
| moderate | 18 | 50 | 41 | 38 |
| high | 37 | 5 | 14 | 19 |
| Myelin content-Peripheral Region | | | | |
| Low | 9 | 23 | 10 | 8 |
| moderate | 28 | 27 | 31 | 28 |
| high | 18 | 5 | 14 | 21 |
| Myelin content-Total | | | | |
| Low | 9 | 23 | 10 | 8 |
| Moderate | 46 | 77 | 72 | 66 |
| High | 55 | 10 | 28 | 40 |
| Sum | 110 | 110 | 110 | 114 |

TABLE 22
Histopathology Scores-Summary and Statistical Analysis

| Myelin | | 2D2- | 2D2- | 2D2- |
|---|---|---|---|---|
| content-Total | WT | vehicle | memantine | sulfasalazine |
| | Percent scores on a per-animal basis | | | |
| Low | 5% | 20% | 10% | 5% |
| Moderate | 40% | 75% | 60% | 55% |
| High | 55% | 5% | 30% | 40% |
| p-value (chi-square | n/a | p < 0.001 | p < 0.001 | p < 0.01 |
| analysis) | | compared | compared | compared to |
| | | to WT | to 2D2 vehicle | 2D2 vehicle |
| | Percent scores on a per-section basis | | | |
| Low | 8% | 21% | 9% | 7% |
| Moderate | 42% | 70% | 65% | 58% |
| High | 50% | 9% | 25% | 35% |
| p-value (chi-square | n/a | p < 0.0001 | p < 0.0001 | p < 0.0001 |
| and Fisher's | | compared | compared | compared to |
| exact test analysis) | | to WT | to 2D2 vehicle | 2D2 vehicle |

There was a highly significant loss of myelin staining intensity in the vehicle-treated 2D2 animals compared to the WT animals (p<0.001 on a per animal basis and p<0.0001 on a per section basis). This indicates that the 2D2 animals had entered a disease state, where significant myelin content in the optic nerve had been lost. 2D2 animals treated with sulfasalazine had a highly significant increase in myelin content compared to the vehicle-treated 2D2 animals (p<0.001 on a per animal basis and p<0.0001 on a per section basis).

2D2 animals treated with memantine also had a highly significant increase in myelin content compared to the vehicle-treated 2D2 animals (p<0.01 on a per animal basis and p<0.001 on a per section basis). This data demonstrates that treatment of the 2D2 mice with sulfasalazine results in a significant increase in myelin content in the optic nerve. This result is consistent with xCT activity being required for full disease pathology. The data demonstrating that memantine—an inhibitor of the glutamate NMDA receptor—also has activity in this model, supports glutamate release through xCT as a pathological mechanism for demyelination. Collectively, this data supports development of sulfasalazine as a potential therapeutic in optic neuritis and other demyelinating diseases.

### Example 17: Enhanced Formulations of Sulfasalazine to Increase Oral Bioavailability

Novel formulations of sulfasalazine containing inhibitors of the ABCG2 transporter were prepared, including a formulation of sulfasalazine that increases the oral bioavailability of the sulfasalazine by at least five-fold in a dog model.

### Preparation of Enhanced Sulfasalazine Formulations

### Sulfasalazine Formulation Exemplar 4: 25% Sulfasalazine:70% PVP VA64:5% TPGS SDD

Spray dried dispersions (SDD) of 25 wt % sulfasalazine, 70 wt % PVP VA64 and 5% TPGS were prepared using a spray drying process as follows. A spray solution was prepared by dissolving 100 mg sulfasalazine and 280 mg PVP VA64 (vinylpyrrolidone-vinyl acetate copolymer (PVP VA64, purchased from BASF as Kollidon® VA 64, Ludwigshafen, Germany) and 20 mg TPGS in 19.6 gm of solvent (95/5 w/w tetrahydrofuran/water), to form a spray solution containing 2 wt % solids. For manufacture of larger amounts (up to 5 gram), larger amounts of these materials were used, but in the same ratio. This solution was spray dried using a spray-dryer, which consisted of an atomizer in the top cap of a vertically oriented stainless steel pipe. The atomizer was a two-fluid nozzle, where the atomizing gas was nitrogen delivered to the nozzle at 70° C. at a flow rate of 31 standard L/min (SLPM), and the solution to be spray dried was delivered to the nozzle at RT. The outlet temperature of the drying gas and evaporated solvent was 31.5° C. Filter paper with a supporting screen was clamped to the bottom end of the pipe to collect the solid spray-dried material and allow the nitrogen and evaporated solvent to escape. The resulting spray dried powder was dried under vacuum overnight.

### Sulfasalazine Formulation Exemplar 5: 25% Sulfasalazine:70% PVP VA64:5% Tween-20 SDD

Spray dried dispersions (SDD) of 25 wt % sulfasalazine, 70 wt % PVP VA64 and 5% TPGS were prepared using a spray drying process as follows. The procedure of sulfasalazine formulation Exemplar 4 was repeated except that 20 mg of Tween-20 was added instead of TPGS. The spray drying conditions were the same as sulfasalazine formulation Exemplar 4. The resulting spray dried powder was dried under vacuum overnight.

### Sulfasalazine Formulation Exemplars 6-9: 25% Sulfasalazine:70% PVP VA64:5% ABCG2 Inhibitor SDD

Additional spray dried dispersions (SDD) of 25 wt % sulfasalazine, 70 wt % PVP VA64 and 5% ABCG2 inhibitor were prepared using a spray drying process as follows. The procedure of sulfasalazine formulation Exemplar 4 was repeated except 20 mg of Brij30, Cremphor EL, Pluronic P85 or Pluronic L21 was used instead of TPGS. The spray drying conditions were the same as sulfasalazine formulation Exemplar 4. The resulting spray dried powder was dried under vacuum overnight.

### Example 18: Characterization of the Enhanced Compositions: PXRD Analysis to Determine Validate Compositions are Amorphous

The six exemplar formulations were analyzed by powder X-ray diffraction (PXRD) using an AXS D8 Advance PXRD measuring device (Bruker, Inc. of Madison, Wis.) as follows. Samples (approximately 100 mg) were packed in Lucite sample cups fitted with Si(511) plates as the bottom of the cup to give no background signal. Samples were spun in the φ plane at a rate of 30 rpm to minimize crystal orientation effects. The X-ray source (KCu_{α}, λ=1.54 Å) was operated at a voltage of 45 kV and a current of 40 mA. Data for each sample were collected over a period of 30 minutes in continuous detector scan mode at a scan speed of 2 seconds/step and a step size of 0.04°/step. Diffractograms were collected over the 2θ range of 4° to 40°. FIG. 19 shows the diffraction pattern of the formulations, revealing an amorphous halo, indicating the sulfasalazine in each of the exemplar formulations was essentially amorphous.

### Determination of Solubility of Reformulated Compounds at Enteric pH:

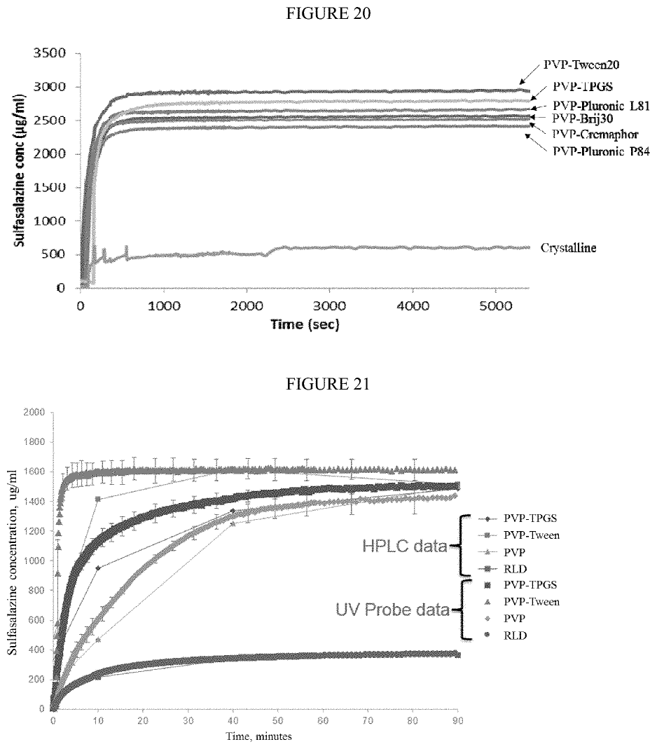

The six exemplar SDDs were tested in an intestinal buffer only dissolution test using a Pion μDissolution in-situ UV-probe instrument. The concentration of total dissolved drug species from the SDDs and crystalline sulfasalazine was measured in phosphate-citrate buffer at pH 5.5 at 37° C. The dose in the test was 3 mg API/mL. FIG. 20 demonstrates that all the SDDs rapidly dissolve to very high concentrations (at least 2300 μg/ml), much higher than observed the crystalline formulation.

Two enhanced SDD formulations containing either TPGS or Tween-20 and the parent amorphous formulation were selected to progress for further development. The enhanced SDD compositions were: 25%/5%/70% sulfasalazine/TPGS/PVP-VA64 (“PVP-TPGS”), 25%/5%/70% sulfasalazine/Tween 20/PVP-VA64 (“PVP-Tween”) and parent SDD formulation was 25% sulfasalazine/75% PVP-VA64 (“PVP”). These formulations were selected on two criteria: 1) Passing the amorphous formulation and initial in vitro dissolution tests described above, and 2) a sufficiently high precedented daily dose as per the FDA. For TPGS, this dose is 300 mg/day, and for Tween 20 it is 56.25 mg/day.

These three SDD formulations were then re-tested in a more stringent two-stage in vitro dissolution test to better mimic actual human dosing. To further mimic actual dosing, the SDD formulations were first encapsulated in Vcaps+ size 00 capsules (Capsugel, Morristown, N.J.) at a drug load of 75 mg API (sulfasalazine) per capsule. As a reference, the on-market formulation of sulfasalazine (Azulfidine tablets, Pfizer, New York, N.Y.) was also tested by encapsulating 75 mg API pieces in the same capsule.

The capsules were initially placed in gastric buffer (pH 2.0 HCl) to a concentration of 3 mg API/mL and then the buffer altered to intestinal buffer (pH 5.5 citrate buffer with 0.5% Fasted and Fed State Simulated Intestinal and Stomach Fluids powder) to a final concentration of 1.5 mg API/mL. The concentration of sulfasalazine in the intestinal buffer was monitored by both HPLC and UV Probe. The monitoring by the UV probe was continuous while for monitoring by HPLC, samples were taken at 10, 40 and 90 minutes after addition of the intestinal buffer solution. FIG. 21 shows the data from the UV and HPLC measurements in graphical format. This data shows that: (1) All SDD formulations had greatly increased solubility in the more stringent two-stage dissolution test that the on-market formulation of sulfasalazine; (2) the PVP-Tween formulation had both the most rapid and the greatest extent of solubility of all the SDD formulations, surpassing both the PVP composition and the PVP-TPGS composition.

### Example 19: Addition of TGPS or Tween-20 Increases the Oral Bioavailability of Sulfasalazine In Vivo

The following experiments demonstrate that addition of TPGS or Tween-20 to an amorphous composition of sulfasalazine results in a significant increase in oral bioavailability compared to administration of the amorphous composition of sulfasalazine alone or the crystalline sulfasalazine in a dog model.

### Animal Dosing

Beagle dogs (approximate weight 8 kg) were fasted overnight. The morning of dosing the animals were administered ˜60 mL of a pre-prepared food slurry (Hills a/d canine food) by gavage, 1 h prior to dose formulation administration. Pentagastrin (10 μg/kg) in a 10% DMA solution was administered (IM) 30 min prior to dosing. Four formulations were tested, each in three dogs, as noted in Table 23.

TABLE 23
Formulations Tested in Dogs

| Name | Description |
|---|---|
| RLD | Sulfasalazine in the reference formulation. This formulation is made from |
| | the on-market Azulfidine tablets obtained from a pharmacy. The pills (500 |
| | mg) were divided into appropriate sized pieces and placed into capsules. |
| PVP | 25% sulfasalazine: 75% PVP-VA64. |
| PVP-Tween | 25% sulfasalazine: 70% PVP-VA64, 5% Tween-20. |
| PVP-TPGS | 25% sulfasalazine: 70% PVP-VA64, 5% TPGS |

For each formulation, size 00 Vcaps+ capsules were loaded with 75 mg of API. Animals were dosed with four (4) capsules, each containing 75 mg API (sulfasalazine), for a total dose of 300 mg API/animal. Normally daily ration was returned 4 hour post-dose. Blood samples (1 ml) were collected via veni-puncture at 10 time points: Blood/Plasma: 0 (predose), 0.25, 0.5, 1, 2, 3, 4, 6, 8 and 24 h postdose. The whole blood samples were placed in a K₂EDTA tube and centrifuged at 2061×g (˜3200 RPM) for 10 minutes at approximately 5° C. The harvested plasma samples were transferred into labeled cryovials and stored at −70±5° C. until analysis.

### Analytical Chemistry:

Blood plasma samples (50 μl) were acidified with 200 μL of extraction buffer (5.25% citric acid/3.3% ammonium phosphate (95:5)) and then extracted with 2.5 ml extraction solvent (methylene chloride/methyl tert-butyl ether (MTBE) (20:80)). Following centrifugation, freezing at −80° C. and drying under nitrogen gas, sample extracts were analyzed and quantitated by high-performance liquid chromatography using a BetaMax Acid column maintained at 40° C. The mobile phase was nebulized using heated nitrogen in a Z-spray source/interface and the ionized compositions were detected and identified using a tandem quadrupole mass spectrometer (MS/MS).

### Analytical Method Qualification:

A reference standard of sulfasalazine (Sigma-Aldrich, Catalog # S0883) was used to generate a standard curve in rat plasma. The assay gave a linear response to concentrations of sulfasalazine from 10 to 10,000 ng/ml (Table 24). Dilution controls showed that samples could be diluted up to 1:100 and give a linear response in the assay.

TABLE 24
Standard Curve Values of Sulfasalazine.

| Nominal | Calculated | |
|---|---|---|
| Concentration | Concentration | % |
| (ng/mL) | (ng/mL) | Deviation |
| 10 | 9.42 | −5.8 |
| 20 | 21.3 | 6.5 |
| 50 | 47.0 | −6.0 |
| 100 | 98.0 | −2.0 |
| 200 | Sample error | Sample error |
| 500 | 465 | −7.0 |
| 1000 | 1090 | 9.0 |
| 2000 | 2110 | 5.5 |
| 5000 | 5180 | 3.6 |
| 10000 | 9660 | −3.4 |

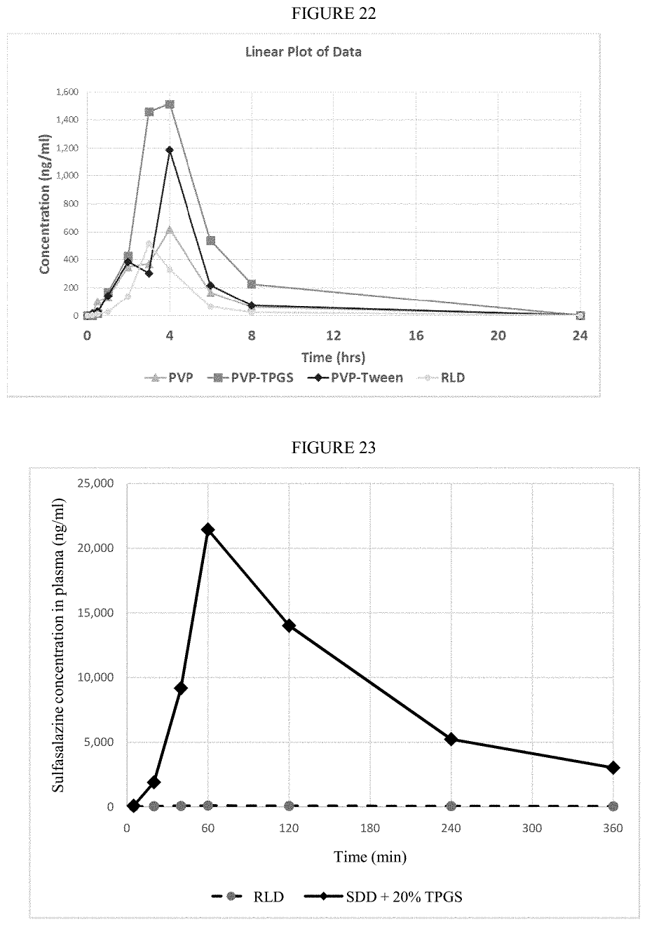

Table 25 shows the mean values for the concentrations of sulfasalazine in the dog plasma, and also the standard deviations (SD) of the measurements. BQL=Below the limit of quantitation (10 ng/ml). The data is graphed in FIG. 22. For the graph, BQL values were assigned a value of 1 ng/ml.

TABLE 25
Mean sulfasalazine levels in plasma and standard deviation

| | Mean Sulfasalazine | | | | | | | |
|---|---|---|---|---|---|---|---|---|
| | Plasma Levels (ng/ml) | | | | Standard Deviation (ng/ml) | | | |
| Time | | | PVP- | PVP- | | | PVP- | PVP- |
| (hrs) | RLD | PVP | Tween | TGPS | RLD | PVP | Tween | TGPS |
| 0 | BQL | BQL | BQL | BQL | 0 | 0 | 0 | 0 |
| 0.25 | BQL | 18 | 16 | BQL | 4 | 7 | 6 | 0 |
| 0.5 | 12 | 102 | 29 | 17 | 3 | 7 | 19 | 17 |
| 1 | 27 | 133 | 142 | 167 | 11 | 35 | 22 | 52 |
| 2 | 139 | 348 | 385 | 426 | 67 | 83 | 183 | 219 |
| 3 | 513 | 370 | 303 | 1456 | 310 | 163 | 191 | 611 |
| 4 | 329 | 619 | 1184 | 1513 | 175 | 217 | 354 | 377 |
| 6 | 65 | 165 | 218 | 538 | 30 | 117 | 56 | 240 |
| 8 | 23 | 63 | 72 | 226 | 7 | 46 | 14 | 131 |
| 24 | BQL | BQL | BQL | BQL | 0 | 5 | 0 | 0 |

Analysis of the pharmacokinetic data is given below in Table 26. Table 26 shows the mean and median area under the curves (AUC), the coefficient of variation (CV), the time to maximum concentration (Tmax), the maximum concentration (Cmax) divided by the AUC, the AUC's relative to the RLD and the statistical significance of the AUC relative to the RLD.

TABLE 26
Analysis of pharmacokinetic data

| | | | | | | | Median |
|---|---|---|---|---|---|---|---|
| | Mean AUC | Median AUC | | Tₘₐₓ ± SD | | Relative | AUC |
| Treatment | (hour · ng/ml) | (hour · ng/ml) | CV | (h) | Cₘₐₓ/AUC | AUC | p-value* |
| RLD | 1517 | 1369 | 29% | 3.3 ± 0.6 | 0.43 | 1 | n/a |
| PVP | 2423 | 2164 | 57% | 4.0 ± 0.0 | 0.22 | 1.8 | 0.19 |
| PVP-TPGS | 7402 | 7362 | 13% | 3.7 ± 0.6 | 0.23 | 4.9 | 0.004 |
| PVP-Tween | 3672 | 3600 | 30% | 4.0 ± 0.6 | 0.32 | 2.4 | 0.088 |

*paired t-test relative to RLD

The data in Table 26 and FIG. 22 demonstrates that: (1) Amorphous compositions of sulfasalazine have higher oral bioavailability than the reference, on-market, crystalline formulation of sulfasalazine; (2) inclusion of an ABCG2 inhibitor, e.g. Tween-20 (Tween) or TPGS, further increased the oral bioavailability beyond that achieved by an amorphous composition alone; and (3) unexpectedly, the PVP-TPGS composition, despite having slower and less absolute solubility than the PVP-Tween composition in the two-stage dissolution test, had much higher oral bioavailability than the PVP-Tween composition (4.9-fold higher than the RLD versus 2.4 fold higher for PVP-Tween). Results demonstrate that the oral bioavailability of sulfasalazine is limited by both solubility at enteric pH and activity of the ABCG2 efflux transporter. The former can be mitigated by making an amorphous composition of sulfasalazine and the latter by including an ABCG2 inhibitor in the formulation. Results demonstrate that both methods can be used simultaneously to make a composition of sulfasalazine with increased oral bioavailability.

### Example 20: Addition of TGPS to 20% Wt/Wt Dramatically and Unexpectedly Increases the Oral Bioavailability of Sulfasalazine In Vivo

The following experiments demonstrate that addition of TPGS to 20% by weight to an amorphous composition of sulfasalazine results in a dramatic and unexpected increase in oral bioavailability compared to administration of the crystalline, on-market (“RLD”) sulfasalazine in a rat model.

### Animal Dosing

Sprague-Dawley rats (approximate weight 200 g) were fasted overnight. The animals were dosed by oral gavage, using the Torpac dosing system with the formulations placed into EL gelatin capsules (Torpac, Inc., Fairfield, N.J.). Two formulations were tested, as noted in Table 27. The first formulation was the reference formulation, made from the on-market Azulfidine tablets obtained from a pharmacy.

The second formulation was a mixture of the 25% sulfasalazine: 75% PVP-VA64 spray-dried dispersion (“SDD”) mixed with 20% TPGS (wt/wt; Isochem, Vert-le-Petit, France; Lot #138917). The SDD and TPGS were mixed with a mortar and pestle for approximately 3 minutes. Mixing in this manner will, among other effects and without limiting the scope of this invention, ensure a more complete and even mixing of the sulfasalazine SDD and TPGS.

TABLE 27
Formulations Tested in Rats

| Name | Description |
|---|---|
| RLD | Sulfasalazine in the reference formulation. This formulation is made |
| | from the on-market Azulfidine tablets obtained from a pharmacy. The |
| | pills (500 mg) were divided into appropriate sized pieces and placed into |
| | capsules. |
| SDD + 20% | 25% sulfasalazine: 75% PVP-VA64 with 20% TPGS (relative to the |
| TPGS | weight of SDD) added |

For each formulation, one or two size EL capsules were loaded with the formulation, such that the total dose each rat received (in either one or two capsules) totaled 10 mg API, e.g. sulfasalazine, per rat. Normally daily ration was returned 1 hour post-dose. Blood samples (0.25 ml) were collected via saphenous veni-puncture at 7 time points: 5, 20, 40, 60, 120, 240 and 360 min post dose. The whole blood samples were placed in a K₂EDTA tube and centrifuged at 2061×g (˜5000 RPM) for 5 minutes at approximately 5° C. The harvested plasma samples were transferred into labeled cryovials and stored at −70±5° C. until analysis.

### Analytical Chemistry:

The treatment of the blood samples and the analytical chemistry was performed as described in Example 19, except conditions were optimized using rat plasma as the matrix instead of dog plasma.

### Analytical Method Qualification:

A reference standard of sulfasalazine (Sigma-Aldrich, Catalog # S0883) was used to generate a standard curve in rat plasma. The assay gave a linear response to concentrations of sulfasalazine from 10 to 10,000 ng/ml (for example of assay linearity, see Table 24). When necessary, samples were diluted 1:10 to remain in the linear portion of the assay. Dilution controls showed that samples could be diluted up to 1:100 and give a linear response in the assay.

Table 28 shows the number of rats tested with the formulation (n), the mean values for the concentrations of sulfasalazine in the rat plasma and also the standard deviations (SD) of the measurements. The data are graphed in FIG. 23.

TABLE 28
Mean sulfasalazine levels in plasma and standard deviation

| | Mean Sulfasalazine Plasma | | Standard Deviation | |
|---|---|---|---|---|
| | Levels (ng/ml) | | (ng/ml) | |
| | | SDD + 20% | | SDD + 20% |
| Time | RLD | TPGS | RLD | TPGS |
| (min) | (n = 9) | (n = 5) | (n = 9) | (n = 5) |
| 5 | 13 | 81 | 8 | 107 |
| 20 | 53 | 1903 | 53 | 691 |
| 40 | 75 | 9176 | 57 | 2776 |
| 60 | 103 | 21440 | 86 | 3828 |
| 120 | 70 | 14010 | 53 | 3884 |
| 240 | 59 | 5233 | 46 | 1699 |
| 360 | 49 | 3023 | 27 | 659 |

Analysis of the pharmacokinetic data is given below in Tables 29a and 29b. Table 29a shows the mean area under the curves (AUC) for each interval and the difference compared to the RLD in percent. The increase in oral bioavailability between the reference formulation and the SDD+20% TPGS ranged from 1000% at 5 minutes to 22,800% at 120 minutes and was still significantly higher at the last time point tested (360 minutes; 9,600%). Overall, the AUC was increased by 15,700% from 0 to 360 minutes following dosing. There was a highly significant difference between the two formulations in AUC for every time interval following the initial 0-5 minute interval. Table 29b shows the Cmax value was increased by 20,800%.

TABLE 29a
Analysis of pharmacokinetic data

| | | | Percent | |
|---|---|---|---|---|
| | | SDD + 20% | difference of | Significance of |
| Time | RLD: Mean | TPGS: Mean | SDD + 20% | SDD + 20% |
| Interval | AUC₀₋₃₆₀ | AUC₀₋₃₆₀ | TPGS | TPGS |
| (mm) | (min · ng/ml) | (min · ng/ml) | versus RLD | versus RLD |
| 0-5 | 20 | 202 | 1,000% | 0.103 |
| 5-20 | 497 | 14,880 | 3,000% | 8.34E−06 |
| 20-40 | 1,104 | 110,792 | 10,000% | 9.79E−07 |
| 40-60 | 1,608 | 306,160 | 19,000% | 2.72E−09 |
| 60-120 | 4,661 | 1,063,500 | 22,800% | 3.72E−07 |
| 120-240 | 6,998 | 1,154,550 | 16,500% | 4.11E−06 |
| 240-360 | 5,172 | 495,300 | 9,600% | 8.16E−08 |
| Total | 20,059 | 3,145,384 | 15,700% | |
| AUC₀₋₃₆₀ | | | | |

TABLE 29b
Analysis of pharmacokinetic data

| | | | Percent |
|---|---|---|---|
| | | | difference |
| | | | of |
| | | SDD + | SDD + |
| | | 20% | 20% TPGS |
| Time (mm) | RLD | TPGS | versus RLD |
| Mean Cmax | 103 | 21,440 | 20,800% |
| (ng/mL) | | | |
| Mean AUC₀₋₃₆₀ | 20,059 | 3,145,384 | 15,700% |
| (min · ng/ml) | | | |

The data in Table 29 and FIG. 23 demonstrates that: (1) inclusion of a TPGS to a concentration by weight of 20% dramatically, significantly and unexpectedly increases the bioavailability of sulfasalazine compared to the RLD.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope. All ranges set forth in this specification include the endpoints provided in those ranges unless clearly indicated otherwise. The entire disclosure of all documents cited throughout this application are incorporated herein by reference.

### REFERENCES

1. Allikmets R, et al. 1997. Characterization of the human ABC superfamily: isolation and mapping of 21 new genes using the expressed sequence tags database. Hum Mol Genet. 5: 1649-55.
2. Bettelli, E. 2003. Myelin oligodendrocyte glycoprotein-specific T cell receptor transgenic mice develop spontaneous autoimmune optic neuritis. J. Exp. Med. 197: 1073-1081.
3. Bogaert, E., et al. 2010. Amyotrophic lateral sclerosis and excitotoxicity: from pathological mechanism to therapeutic target. CNS Neurol. Disord. Drug Targets 9:297-304.
4. Choi, J., et al. 2009 Cellular injury and neuroinflammation in children with chronicintractable epilepsy. J Neuroinflammation 6:38-52.
5. Domercq, M., et al. 2007. System xc− and glutamate transporter inhibition mediates microglial toxicity to oligodendrocytes. J Immunol. 178: 6549-6556.
6. Doyle, L., et al. 1999. A multidrug resistance transporter from human MCF-7 breast cancer cells. Proc Natl Acad Sci USA. 95: 15665-70.
7. Esfahani M., et al. 2012. Memantine for axonal loss of optic neuritis. Graefes Arch Clin Exp Ophthalmol. 250: 863-869.
8. Espey, M., et al. 1998. Extracellular glutamate levels are chronically elevated in brains of LP-BM5-infected mice: A mechanism of retroviral-induced encephalopathy. J Neurochem. 71 2079-2087.
9. Fogal, B., et al. 2007. System x_{c}⁻ activity and astrocytes are necessary for interleukin-1b-mediated hypoxic neuronal injury. J Neurosci. 27 10094-10105.
10. Guan, Y., et al. 2006. Retinal ganglion cell damage induced by spontaneous autoimmune optic neuritis in MOG-specific TCR transgenic mice. J. Neuroimmunol. 178:40-48.
11. Gurney M, et al. 1994. Motor neuron degeneration in mice that express a human Cu/Zn superoxide dismutase mutation. Science 264: 1772-1775.
12. Ilieva, H., et al. 2009. Non-cell autonomous toxicity in neurodegenerative disorders: ALS and beyond. J. Cell Biol. 187: 761-772.
13. Kwan, P., et al. 2011. Drug-Resistant Epilepsy. N Engl J Med 365: 919-926.
14. Lidster, K., et al. 2013. Neuroprotection in a novel mouse model of multiple sclerosis. PLoS. 8: e79188.
15. Lin, L., et al. 2010. The interaction of the neuroprotective compounds riluzole and phenobarbital with AMPA-type glutamate receptors: a patch-clamp study. Pharmacology. 85:54-62.
16. Lincecum, J., et al. 2010. From transcriptome analysis to therapeutic anti-CD40L treatment in the SOD1 model of amyotrophic lateral sclerosis. Nat. Genetics 42: 392-411.
17. Massie, A. et al. 2010. Dopaminergic neurons of system x_{c}⁻ deficient mice are protected against 6-OHDA induced toxicity. FASEB J. December 29. Epub ahead of print.
18. Massie, A. et al. 2008. Time-dependent changes in striatal xCT protein expression in hemi-Parkinson rats. Mol Neurosci. 19 1589-1592.
19. Palop, J and Mucke, L. 2006. A network dysfunction perspective on neurodegenerative diseases. Nature 443 768-773.
20. Peppercorn, M. 1987. Sulfasalazine and related new drugs. J Clin Pharmacol. 27: 260-265.
21. Philips. T., et al. 2011. Neuroinflammation in amyotrophic lateral sclerosis: role of glial activation in motor neuron disease. Lancet Neurol. 10:253-263.
22. Polewski, M., et al. 2016. Increased expression of system xc− in glioblastoma confers an alteredmetabolic state and temozolomide resistance. Mol. Cancer Res. 14: 1229-1242.
23. Qin, S., et al. 2006. System xc− and apolipoprotein E expressed by microglia have opposite effects on the neurotoxicity of amyloid-beta peptide 1-40. J Neurosci. 26 3345-3356.
24. Sato, H., et al. 2004. Transcriptional control of the cystine/glutamate transporter gene by amino acid deprivation. Biochem. Biophys. Res. Comm. 325: 109-116.
25. Schinthal, A. 2012. Endoplasmic reticulum stress: its role in disease and novel prospects for therapy. Scientifica (Cairo). Article ID 857516.
26. Scott S, et al. 2008. Design, power, and interpretation of studies in the standard murine model of ALS. Amyotroph Lateral Scler 9: 4-15.
27. Shin, J., et al. 2012. Concurrent blockade of free radical and microsomal prostaglandin E synthase-1-mediated PGE(2) production improves safety and efficacy in a mouse model of amyotrophic lateral sclerosis. J. Neurochem. 122: 952-961.
28. Sontheimer, H and Bridges, R. 2012. Sulfasalazine for brain cancer fits. Expert Opin. Investig. Drugs. 21: 575-578.
29. Suhs, K. 2014. N-methyl-d-aspartate receptor blockade is neuroprotective in experimental autoimmune optic neuritis. J Neuropathol Exp Neurol. 73:507-518.
30. Takeuchi, S. et al. (2014). Sulfasalazine and temozolomide with radiation therapy for newly diagnosed glioblastoma. Neurology India 62: 42-47.
31. Talla, V., et al. 2013. Noninvasive assessments of optic nerve neurodegeneration in transgenic mice with isolated optic neuritis. Invest Ophthalmol Vis Sci. 54:4440-4450.
32. Watkinson, G. 1986. Sulphasalazine: a review of 40 years' experience. Drugs. 32: Suppl 1:1-11.
33. Weaver, A., et al. 1999. Improved gastrointestinal tolerance and patient preference of enteric-coated sulfasalazine versus uncoated sulfasalazine tablets in patients with rheumatoid arthritis. J. Clin. Rheumatology. 5: 193-200.
34. Yamasaki, Y., et al. 2008. Pharmacogenetic characterization of sulfasalazine disposition based on NAT2 and ABCG2 (Bcrp) gene polymorphisms in humans. Clin Pharmacology Therapeutics. 84:95-103.
35. Zaher, H., et al. 2005. Breast cancer resistance protein (Bcrp/abcg2) is a major determinant of sulfasalazine absorption and elimination in the mouse. Mol. Pharmaceutics 3: 55-61.

1.-17. (canceled)

18. A method for the treatment of neurodegenerative disease selected from P-MS, ALS, Parkinson's disease, Alzheimer's disease, epilepsy and other seizure disorders, neuropathic pain, traumatic brain injury, Huntington's disease, ischemic stroke, Rett Syndrome, Frontotemporal Dementia, HIV-associated Dementia and Alexander disease, the method comprising:
administering to a patient a pharmaceutical composition comprising a therapeutically effective amount of 2-hydroxy-5-[[4-[(2-pyridinylamino)sulfonyl]phenyl]azo]benzoic acid (sulfasalazine), an ATP-binding cassette sub-family G member 2 inhibitor (ABCG2 inhibitor); and a pharmaceutically acceptable excipient;
wherein the administration of the pharmaceutical composition provides an increase of at least 200% in the bioavailability of sulfasalazine when compared to an RLD.

19. The method of claim 18, wherein the ABCG2 inhibitor is TPGS or Tween-20.

20. The method of claim 18, wherein the pharmaceutically acceptable excipient is PVP-VA64.

21.-28. (canceled)
